(19) United States
(12) Patent Application Publication
(10) Pub. No.: US 2016/0251651 A1
(43) Pub. Date: Sep. 1, 2016

(54) CELL FREE CLONING OF NUCLEIC ACIDS

(71) Applicant: TWIST BIOSCIENCE CORPORATION, San Francisco, CA (US)

(72) Inventors: William BANYAI, San Francisco, CA (US); Bill James PECK, Santa Clara, CA (US); Andres FERNANDEZ, San Francisco, CA (US); Siyuan CHEN, San Mateo, CA (US); Pierre INDERMUHLE, Berkeley, CA (US)

(21) Appl. No.: 15/156,134

(22) Filed: May 16, 2016

Related U.S. Application Data
(63) Continuation of application No. PCT/US15/43605, filed on Aug. 4, 2015.
(60) Provisional application No. 62/033,587, filed on Aug. 5, 2014.

(51) Int. Cl. C12N 15/10

(52) U.S. Cl. CPC C12N 15/1093

(57) ABSTRACT

Methods and devices for cell-free sorting and cloning of nucleic acid libraries are provided herein.

### CROSS-REFERENCE

This application claims the benefit of U.S. Provisional Application No. 62/033,587 filed Aug. 5, 2014, which is herein incorporated by reference in its entirety.

### BACKGROUND

Highly efficient chemical gene synthesis with high fidelity and low cost has a central role in biotechnology and medicine, and in basic biomedical research. While various methods are known for the synthesis of relatively short fragments in a small scale, these techniques often suffer from scalability, automation, speed, accuracy, and cost. One obstacle in this area is the efficient sorting and cloning of error free nucleic acid sequences.

### BRIEF SUMMARY

In some embodiments, a method for nucleic acid sorting is provided, the method comprising providing a sample with a plurality of circularized nucleic acids, partitioning such that on average there are about 0.1 to 10 circularized nucleic acids from the plurality of circularized nucleic acids per fraction, and amplifying the partitioned circularized nucleic acids in the presence of a random primer to generate a plurality of amplicon nucleic acids, wherein the random primer comprises 4 to 8 bases in length. In some embodiments, each circularized nucleic acid in the plurality of circularized nucleic acids is double-stranded. In some embodiment, forming each circularized nucleic acid in the plurality of circularized nucleic acids comprises ligating an adapter sequence to a sticky end of a non-circularized nucleic acid, wherein the adapter sequence links a 5′ end to a 3′ end of the non-circularized nucleic acid. In some embodiments, the sticky end is a 3′ overhang of the non-circularized nucleic acid. In some embodiments, the sticky ends are formed on both the 3′ end and the 5′ end of the non-circularized nucleic acid. In some embodiments, the adapter sequence comprises at least one sticky end. In some embodiments, the at least one sticky end of the adapter sequence comprises a 3′ overhang or a 5′ overhang. In some embodiments, a strand of the adapter sequence lacks a 5′ phosphate. In some embodiments, forming each circularized nucleic acid in the plurality of circularized nucleic acids comprises providing a sample with a plurality of non-circularized nucleic acids, forming sticky ends at each end of each of the non-circularized nucleic acids, wherein the sticky ends comprise 3′ overhangs 4 to 10 bases in length, ligating the sticky ends to form a plurality of double-stranded circularized nucleic acids. In some embodiments, the 3′ overhangs are 4 bases in length. In some embodiments, the plurality of double-stranded circularized nucleic acids comprise a gap 1 to 5 bases in length. In some embodiments, the gap length is 1 base. In some embodiments, the plurality of circular double-stranded nucleic acids is formed by providing a sample with a plurality of non-circularized nucleic acids, amplifying the plurality of non-circularized nucleic acids with a first primer comprising a 5′ phosphate and a second primer lacking a 5′ phosphate to form a double-stranded amplification product, and ligating one strand of the double-stranded amplification product. In some embodiments, partitioning comprises diluting such that on average there are about 0.5 to 2 of the circularized nucleic acids per fraction. In some embodiments, partitioning comprises diluting such that on average there is about 1 circularized nucleic acid per fraction. In some embodiments, amplifying comprises PCR, MDA, or Rolling Circle Amplification (RCA). In some embodiments, the method comprises sequencing nucleic acids from one or more fractions. In some embodiments, partitioning comprises diluting to a concentration of about 1.5 to 17 circularized nucleic acids per 1 μl of solution. In some embodiments, the concentration of the sample is measured prior to partitioning. In some embodiments, the circularized nucleic acids are heat denatured prior to amplification. In some embodiments, the sample comprises at least 2, 3, 4, 5, 6, 7, 8, 9, 10, 15, 20, 25, 30, 40, 50, or 100 circularized nucleic acids at least 500 bases in length. In some embodiments, amplifying results in at least 1, 2, 3, 4, 5, 6, 7, 8, 9, 10, 15, 20, 25, 30, 40, 50, or 100 copies of the plurality of circularized nucleic acids. In some embodiments, the plurality of circularized nucleic acids comprises nucleic acids that differ in at least 1, 2, 3, 4, 5, 6, 7, 8, 9, 10, 15, 20, 25, 30, 40, 50, or 100 bases. In some embodiments, each circular nucleic acid of the plurality of circularized nucleic acids is at least 250, 500, 750, 1000, 1500, or 2000 nucleotides in length. In some embodiments, the random primer is 6 bases in length. In some embodiments, adapter sequence comprises a central double-stranded region about 20 to about 30 bases in length and a 3′ overhang on each end about 8 or about 9 bases in length. In some embodiments, the adapter sequence is about 22 bases in length. In some embodiments, each non-circularized nucleic acid encodes for a gene sequence.

In some embodiments, a method for nucleic acid sorting is provided, the method comprising providing a plurality of circular double-stranded nucleic acids, wherein a first strand of the plurality of circular double-stranded nucleic acids is a complete circle and a second strand of the plurality of circular double-stranded nucleic acids comprises a gap or a nick, diluting the plurality of circular double-stranded nucleic acids to a concentration of less than 100 nM, extending the second strand of the plurality of circular double-stranded nucleic acids in a first amplification reaction using the first strand as a template, thereby forming a plurality of amplicon nucleic acids comprising a plurality of copies of the first strand of the plurality of circular double-stranded nucleic acids, and partitioning such that on average there are 0.1 to 10 amplicon nucleic acids per fraction. In some embodiments, the plurality of circular double-stranded nucleic acids is formed by providing a sample with a plurality of non-circularized nucleic acids, and adding an adapter sequence to each nucleic acid of the plurality of non-circularized nucleic acids, wherein the adapter sequence links a 5′ end to a 3′ end of each nucleic acid of the plurality of nucleic acids. In some embodiments, the sample comprises at least 2, 3, 4, 5, 6, 7, 8, 9, 10, 15, 20, 25, 30, 40, 50, or 100 nucleic acids at least 500 bases in length. In some embodiments, the method comprises forming at least 2, 3, 4, 5, 6, 7, 8, 9, 10, 15, 20, 25, 30, 40, 50, or 100 circular nucleic acids for each nucleic acid in the plurality of nucleic acids. In some embodiments, the gap or nick is formed at a juncture of the adapter sequence and each nucleic acid of the plurality of non-circularized nucleic acids. In some embodiments, forming the plurality of circular double-stranded nucleic acids comprises forming sticky ends at the ends of each of the non-circularized nucleic acids. In some embodiments, the sticky ends comprise a 3′ overhang. In some embodiments, the adapter sequence comprises at least one sticky end. In some embodiments, the at least one sticky end of the adapter sequence comprises a 3′ overhang. In some embodiments, one of the strands of the adapter sequence lacks a 5′ phosphate. In some embodiments, the plurality of circular double-stranded nucleic acids is formed by providing a sample with a plurality of non-circularized nucleic acid, forming sticky ends at each end of each of the non-circularized nucleic acids, wherein the sticky ends comprise 3′ overhangs 4 to 10 bases in length, and ligating the sticky ends. In some embodiments, the 3′ overhangs are 4 bases in length. In some embodiments, the gap length is 1 to 5 bases. In some embodiments, the gap length is 1 base. In some embodiments, the plurality of circular double-stranded nucleic acids is formed by providing a sample with a plurality of non-circularized nucleic acids, amplifying the plurality of non-circularized nucleic acids with a first primer comprising a 5′ phosphate and a second primer lacking a 5′ phosphate to form a double-stranded amplification product, and ligating one strand of the double-stranded amplification product. In some embodiments, dilution of the plurality of circular double-stranded nucleic acids is to a concentration of less than about 100 nM, 10 pM, 1 pM, 500 fM, 100 fM, 10 fM, or 5 fM prior to extending the second strand of each of the circular nucleic acids. In some embodiments, dilution of the plurality of circular double-stranded nucleic acids is to a concentration of less than about 500 fM prior to extending the second strand of each of the circular nucleic acids. In some embodiments, dilution of the plurality of circular double-stranded nucleic acids is to a concentration of less than about 100 fM prior to extending the second strand of each of the circular nucleic acids. In some embodiments, partitioning comprises diluting the plurality of amplicon nucleic acids by a ratio of at least 1:10,000. In some embodiments, partitioning comprises diluting the plurality of amplicon nucleic acids to about 0.3 to 1.5 amplicon nucleic acids per fraction. In some embodiments, partitioning comprises diluting the plurality of amplicon nucleic acids to about 1.2 amplicon nucleic acids per fraction. In some embodiments, partitioning comprises diluting the plurality of amplicon nucleic acids to about 1.0 amplicon nucleic acids per fraction. In some embodiments, partitioning comprises diluting the plurality of amplicon nucleic acids to a concentration of about 1-200 molecules per 1 μl of solution. In some embodiments, partitioning comprises diluting the plurality of amplicon nucleic acids to a concentration of about 15-17 molecules per 1 μl of solution. In some embodiments, the first amplification reaction comprises PCR, MDA, or Rolling Circle Amplification (RCA). In some embodiments, the method comprises a second amplification reaction, wherein the second amplification reaction is performed after partitioning. In some embodiments, the method further comprises sequencing nucleic acids from one or more fractions. In some embodiments, the plurality of amplicon nucleic acids comprises at least 1, 2, 3, 4, 5, 6, 7, 8, 9, 10, 15, 20, 25, 30, 40, 50, or 100 copies of the first strand of one of the circular nucleic acids. In some embodiments, the plurality of circular double-stranded nucleic acids comprises nucleic acids that differ in at least 1, 2, 3, 4, 5, 6, 7, 8, 9, 10, 15, 20, 25, 30, 40, 50, or 100 bases. In some embodiments, the gap or nick is at least 1, 2, 3, 4, 5, 6, 7, 8, 9, 10, 15, 20, or 25 nucleotides long. In some embodiments, each nucleic acid of the plurality of amplicon nucleic acids is single-stranded. In some embodiments, the gap has a length about 1 to 5 bases. In some embodiments, each circular nucleic acid of the plurality of circular double-stranded nucleic acids is at least about 500, 750, 1000, 1500, or 2000 nucleotides in length. In some embodiments, the circular double-stranded nucleic acids are heat denatured prior to amplification. In some embodiments, adapter sequence comprises a central double-stranded region about 20 to about 30 bases in length and a 3′ overhang on each end about 8 or about 9 bases in length. In some embodiments, the adapter sequence is about 22 bases in length. In some embodiments, each non-circularized nucleic acid encodes for a gene sequence.

In some embodiments, a method for nucleic acid sorting is provided, the method comprising forming a plurality of circular nucleic acids by a ligation reaction, wherein ligation comprises joining a non-circularized nucleic acid and two adapter sequences, wherein each of the adapter sequences encodes for a hairpin secondary structure, diluting the plurality of circular nucleic acids to a concentration of at most 1 nM, amplifying the circularized plurality of nucleic acids in the presence of a primer having sequence complementary to one of the two adapter sequences, and partitioning the amplification reaction such that on average there are 0.1 to 10 amplicon nucleic acids per fraction. In some embodiments, the plurality of circular nucleic acids is diluted to a concentration of less than about 100 pM, 10 pM, or 1 pM prior to amplification. In some embodiments, the plurality of circular nucleic acids is diluted to a concentration of about of 1 pM prior to amplification. In some embodiments, partitioning is performed such that there are on average about 0.3 to 1.5 amplicon nucleic acids per fraction. In some embodiments, partitioning is performed such that there is on average about 1 amplicon nucleic acids per fraction. In some embodiments, the plurality of circular nucleic acids comprises generating sticky ends at a 3′ end and a 5′ end of the non-circularized nucleic acid. In some embodiments, the sticky ends comprise a 3′ overhang. In some embodiments, each of the two adapter sequences comprises at least one sticky end. In some embodiments, the at least one sticky end comprises a 3′ overhang. In some embodiments, amplifying comprises Rolling Circle Amplification (RCA). In some embodiments, the method further comprises sequencing nucleic acids from one or more fractions. In some embodiments, the plurality of circular nucleic acids comprises at least 2, 3, 4, 5, 6, 7, 8, 9, 10, 15, 20, 25, 30, 40, 50, or 100 nucleic acids at least 500 bases in length. In some embodiments, the plurality of circular nucleic acids comprises nucleic acids that differ in at least 1, 2, 3, 4, 5, 6, 7, 8, 9, 10, 15, 20, 25, 30, 40, 50, or 100 bases. In some embodiments, the each circular nucleic acid in the plurality of circular nucleic acids is at least 250, 500, 750, 1000, 1500, or 2000 nucleotides in length. In some embodiments, each of the amplicon nucleic acid binds to the surface of a well. In some embodiments, each non-circularized nucleic acid encodes for a gene sequence.

In some embodiments, a method for nucleic acid purification is provided, the method comprising aliquoting packages of amplicons of at least two different nucleic acid sequences in a sample into partitions such that each partition receives on average 0.001 to 2 packages of amplicons wherein each package of amplicons comprises amplicons from a single one of the at least two different nucleic acid sequences. In some embodiments, each partition comprises a droplet, bead, well, resolved features on a substrate, or discrete volumes in a gel. In some embodiments, the substrate comprises a patterned surface, comprising active and passive areas, wherein the active areas are coated with a moiety to aid retention of the packages and the passive areas are not. In some embodiments, the active areas hold at most one package. In some embodiments, the partitions comprise droplets in an emulsion and wherein the droplets in the emulsion are sorted. In some embodiments, the droplets in the emulsion are sorted by flow cytometry. In some embodiments, the partitions further comprise a nucleic acid dye. In some embodiments, the nucleic acid dye comprises N′,N′-dimethyl-N-[4-[(E)-(3-methyl-1,3-benzothiazol-2-ylidene)methyl]-1-phenylquinolin-1-ium-2-yl]-N-propylpropane-1,3-diamine. In some embodiments, the method further comprises performing nucleic acid amplification within the partitions. In some embodiments, the nucleic acid amplification comprises PCR, MDA, or RCA. In some embodiments, the number of packages of amplicons for aliquoting is at least 2, 3, 4, 5, 6, 7, 8, 9, 10, 15, 20, 50, 75, or 100. In some embodiments, the packages of amplicons are of at least 2, 3, 4, 5, 6, 7, 8, 9, 10, 15, 20, 50, 75, or 100 different nucleic acid sequences. In some embodiments, the packages of amplicons are formed by rolling circle amplification (RCA). In some embodiments, the partitions further comprise at least one primer. In some embodiments, the partitions further comprise a DNA polymerase. In some embodiments, each of the partitions is located within a well about 1.0 to 2.0 mm in diameter and having an internal depth of about 300 to 500 microns.

In some embodiments, a gene library is provided, wherein the gene library is generated by any of the methods described herein.

### INCORPORATION BY REFERENCE

All publications, patents, and patent applications disclosed herein are incorporated by reference to the same extent as if each individual publication, patent, or patent application was specifically and individually indicated to be incorporated by reference. In the event of a conflict between a term disclosed herein and a term in an incorporated reference, the term herein controls.

### BRIEF DESCRIPTION OF THE DRAWINGS

FIG. 1: Depicts a first exemplary workflow for cell free sorting.

FIG. 2: Depicts an exemplary workflow for circularization of a double-stranded target nucleic acid.

FIG. 3: Depicts a second exemplary workflow for cell free sorting.

FIG. 4: Depicts a third exemplary workflow for cell free sorting

FIGS. 5A-5C: FIGS. 5A-5C present a diagram of steps demonstrating an exemplary process workflow for gene synthesis as disclosed herein.

FIGS. 6A-6C: FIGS. 6A-6C depict an embodiment of a process for gene synthesis as disclosed herein.

FIG. 7: Depicts an electrophoresis digital trace for target nucleic acids amplified with uracil containing primers.

FIG. 8: Depicts a sequence alignment map for PCR products amplified from a partitioned fraction number 1.

FIG. 9: Depicts a sequence alignment map for PCR products amplified from a partitioned fraction number 2.

FIG. 10: Depicts a sequence alignment map for PCR products amplified from a partitioned fraction number 3.

FIG. 11: Depicts a sequence alignment map for PCR products amplified from a partitioned fraction number 4.

FIG. 12: Depicts a sequence alignment map for PCR products amplified from a partitioned fraction number 5.

FIG. 13: Depicts a sequence alignment map for a sample of RCA products prior to partitioning into fractions.

FIG. 14: Depicts a sequence alignment map for a 2-component blended sample of target nucleic acids prior to clonal sorting.

FIGS. 15A-15D: FIGS. 15A-15B depict electrophoresis gels showing the presence or absence of nucleic acids amplified from partitioned fractions comprising, on average, an expected 1.2 parent nucleic acids per fraction. FIGS. 15C-15D depict electrophoresis gels showing the presence or absence of nucleic acids amplified from partitioned fractions comprising, on average, an expected 0.6 parent nucleic acids per fraction.

FIG. 16: Depicts a sequence alignment map of nucleic acids amplified from a partitioned fraction shown in FIG. 15C.

FIG. 17: Depicts a sequence alignment map of nucleic acids amplified from a partitioned fraction shown in FIG. 15C.

FIGS. 18A-18B: FIGS. 18A-18B depict electrophoresis gels showing the presence or absence of clonally sorted nucleic acids into fractions comprising single molecule RCA amplification products.

FIGS. 19A-19B: FIGS. 19A-19B depict electrophoresis gels showing PCR products amplified from products of a RCA reaction performed in nanowell partitions.

FIG. 20: Depicts an electrophoresis gel showing target nucleic acids circularized by hybridization and ligation to hairpins.

FIGS. 21A-21B: FIG. 21A depicts an electrophoresis gel showing nucleic acid amplification products of partitioned fractions, where each partitioned fraction had, on average, 10 molecules of parent DNA that were amplified by RCA followed by PCR. FIG. 21B depicts an electrophoresis gel showing nucleic acid amplification products of partitioned fractions, where each partitioned fraction had, on average, 1 molecules of parent DNA that were amplified by RCA followed by PCR.

FIG. 22: Depicts a sequence alignment map of nucleic acid amplification products of a partitioned fraction number 2 shown in FIG. 21B.

FIG. 23: Depicts a sequence alignment map of nucleic acid amplification products of a partitioned fraction number 3 shown in FIG. 21B

FIG. 24: Depicts a sequence alignment map of nucleic acid amplification products of a partitioned fraction number 6 shown in FIG. 21B

FIG. 25: Depicts a sequence alignment map of nucleic acid amplification products of a partitioned fraction number 7 shown in FIG. 21B.

FIG. 26: Depicts a sequence alignment map of nucleic acid amplification products of a partitioned fraction number 8 shown in FIG. 21B

FIG. 27: Depicts a sequence alignment map of nucleic acid amplification products of a partitioned fraction number 9 shown in FIG. 21B

FIG. 28: Depicts a sequence alignment map of nucleic acid amplification products of a partitioned fraction number 10 shown in FIG. 21B

FIG. 29: Depicts a sequence alignment map of nucleic acid amplification products of a partitioned fraction number 11 shown in FIG. 21B

FIG. 30: depicts a sequence alignment map of nucleic acid amplification products of a partitioned fraction number 12 shown in FIG. 21B

FIGS. 31A-31C: FIG. 31A depicts an electrophoresis gel showing target nucleic acids circularized by sticky end self-ligation. FIG. 31B depicts a chart showing RCA amplification of target nucleic acids circularized by sticky end self-ligation. FIG. 31C depicts an electrophoresis gel showing target nucleic acids circularized by blunt end self-ligation.

FIG. 32: Illustrates an example of a computer system.

FIG. 33: Depicts a block diagram illustrating exemplary architecture of a computer system.

FIG. 34: Depicts a diagram demonstrating a network configured to incorporate a plurality of computer systems, a plurality of cell phones and personal data assistants, and Network Attached Storage (NAS).

FIG. 35: Depicts a block diagram of a multiprocessor computer system using a shared virtual address memory space.

### DETAILED DESCRIPTION

The present disclosure provides methods for nucleic acid sorting and cloning of heterogeneous populations of nucleic acids in a cell-free environment. Further provided are methods and systems for the synthesis of oligonucleic acids with low error rates, where the synthesized products, or assembled products thereof, are clonally sorted using cell-free sorting.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Reference herein to “target” refers to a particular nucleic acid molecule. Reference herein to a “sample” refers to a source material containing a heterogeneous population of nucleic acids. Reference herein to an “amplicon” refers to a product of a nucleic acid amplification reaction.

Cell-Free Sorting and Cloning of Nucleic Acids

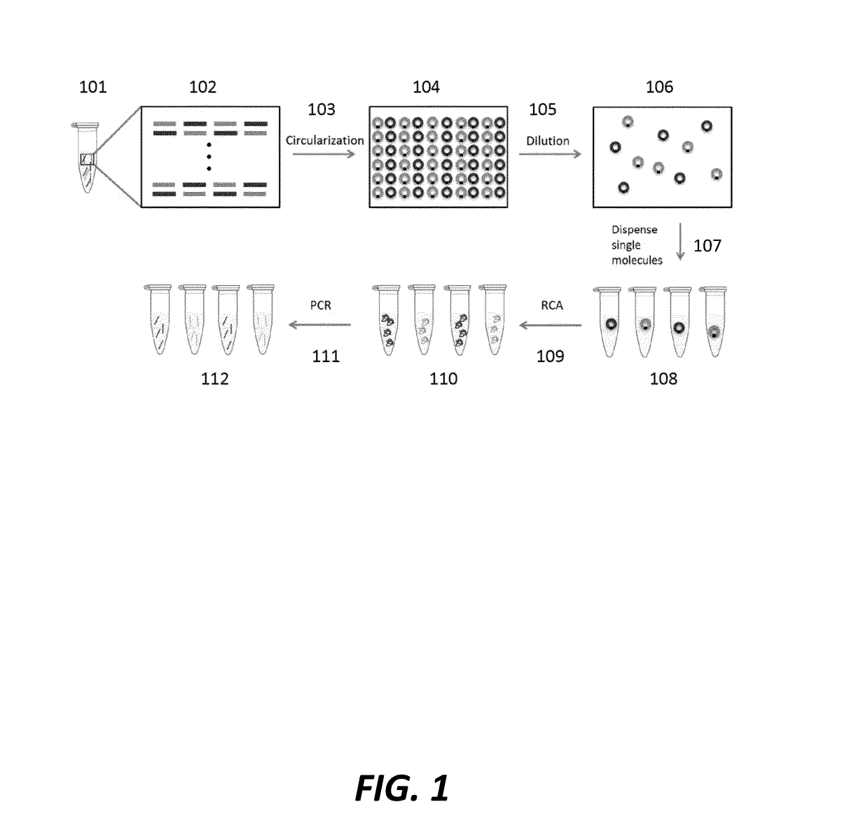

A first example of cell-free sorting and cloning is depicted in FIG. 1. A starting sample 101 includes a heterogeneous population of double-stranded target nucleic acids 102. The heterogeneous population of double-stranded target nucleic acids is circularized 104, followed by dilution 105 to generate a pool 106 for dispensing 107 into partitions 108 where each partition comprises on average about 1 circularized double-stranded nucleic acid. In some cases, circularized nucleic acid is heat denatured prior to amplification. A rolling circle amplification (RCA) reaction 109 is performed with the partitioned circularized nucleic acids to generate amplicons 110. A second round of amplification, for example with a polymerase chain reaction (PCR) 111 is performed to generate additional copies of a particular clonal population 112. In some cases, sequencing of amplification product occurs after the RCA reaction 109. In some cases, sequencing of amplification product occurs after the PCR step 111. Sequencing data corresponding to clonal populations is compared to that of predetermined sequence(s).

The heterogeneous population of nucleic acids 101 includes one or more of the nucleic acids comprising a sequence that is different from one or more other nucleic acids within the population. In some cases, the population of nucleic acids comprises at least about 2, 3, 4, 5, 6, 7, 8, 9, 10, 15, 20, 25, 30, 40, 50, 100 or more nucleic acids having a sequence that is different from another nucleic acid in the population. Sources for difference in nucleic acid sequence between target nucleic acids in a sample population include, for example, a mutation, insertion, deletion or combination thereof. Exemplary nucleic acid lengths for target sequence include, without limitation, about or at least about 100, 150, 200, 250, 300, 350, 400, 500, 600, 700, 800, 900, 1000, 1200, 1400, 1600, 1800, 2000, 2200, 2400, 2600, 2800, 3000, 3500, 4000, 4500, 5000, 5500, 6000, 6500, 7000, 7500, 8000, 8500, 9000, 9500, 10000 or more bases in length. Exemplary methods for circularization of nucleic acids include, without limitation, (1) ligation with one or more nucleic acid adapters or plasmids, to generate double-stranded, circularized nucleic acid, (2) self-ligation of a double-stranded nucleic acid sequence to generate a circularized nucleic acid, and (3) ligation with one or more hairpin molecules to generate single-stranded, circularized nucleic acid. While the workflow in FIG. 1 refers to generation of circularized double-stranded nucleic acid, in some cases a circularized single-stranded nucleic acid is used, for example, in the hairpin arrangement.

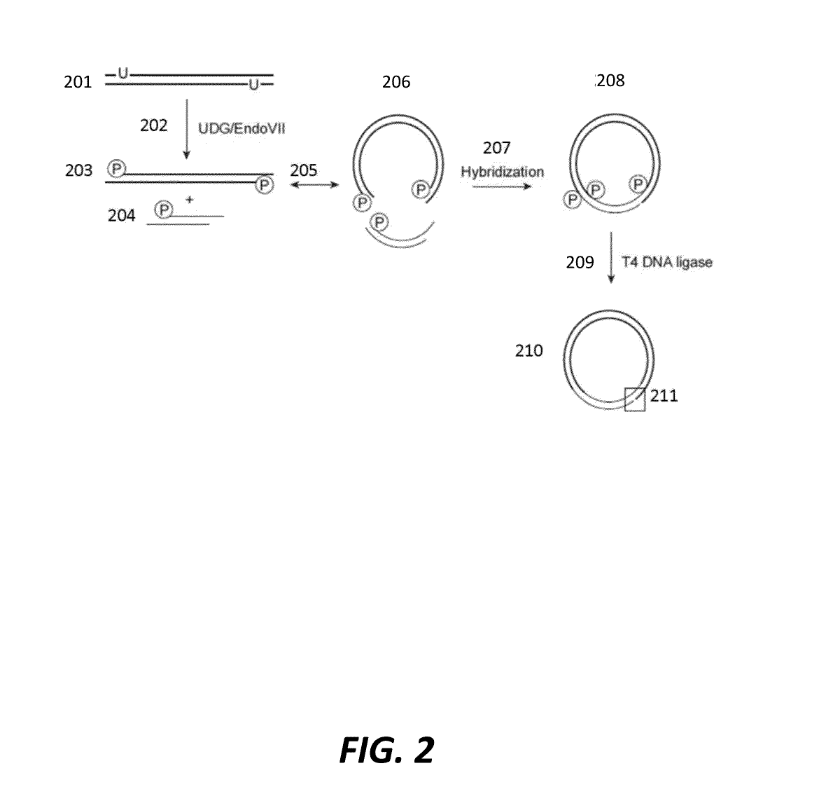

An example workflow for ligating double-stranded nucleic acid to an adapter sequence is depicted in FIG. 2. A double-stranded nucleic acid 201 comprises a uracil base near the 5′ end of the first strand and a uracil base near the 5′ end of the second strand. In some cases, uracil bases are incorporated into the population of nucleic acids to be sorted using primers comprising one or more uracil bases. In other cases, the uracil is incorporated by nucleic acid synthesis. Depending on the desired overhang, a uracil base is incorporated near the 5′ or 3′ end of a strand such that it is located about 1, 2, 3, 4, 5, 6, 7, 8, 9, 10, 11, 12, 13, 14, 15, 16, 17, 18, 19, 20 or more bases from the end of the strand. In some embodiments, a double-stranded target nucleic acid comprises one or more overhangs for ligation to an adapter, for example, one or two 3′ overhangs, one or two 5′ overhangs, or a 3′ and 5′ overhang. In some embodiments, the adapter is a double-stranded nucleic acid comprising one or more overhangs, for example, one or two 3′ overhangs, one or two 5′ overhangs, or a 3′ and 5′ overhang. In some embodiments, a strand of a double-stranded adapter comprises a 5′ phosphate group for ligation to a 3′ end of a strand of a double-stranded target nucleic acid. In some instances, an adapter comprises between about 20 bases and about 150 bases. In some cases, an adapter comprises about 20, 25, 30, 35, 40, 45, 50, 55, 60, 65, 70, 75, 80, 85, 90, 95 100, 150, 200 or more bases.

As shown in FIG. 2, treating double-stranded nucleic acid having 5′ uracil bases with Uracil DNA glycosylase (UDG) and Endonuclease VIII (EndoVIII) 202 results in generation of 3′ overhangs (sticky ends) 203. An adapter sequence 204 is mixed with the cleaved double-stranded nucleic acid 205. Interaction between the two molecules 206 results in hybridization 207. After a ligation reaction 209, circular double-stranded nucleic acid is formed 210. In this example, the adapter sequence is designed with only a single 5′ phosphate group, preventing a complete circle from forming after the ligase reaction for the second strand of nucleic acid 211. In some cases, the adapter, the target nucleic acid, or both are constructed or treated such that when the adapter and the target are ligated, only one of each strand of adapter and target DNA can ligate to form a continuous circle; and the other strands of the adapter and target DNA can only circularize upon hybridization to the continuous circle. In such cases, the second strand comprises phosphorothioated bonds between bases at its 5′ end so that upon exonuclease digestion of a sample of self-ligated target nucleic acids, the discontinuous strand resists digestion. In other cases, the adapter sequence contains 5′ phosphates at both ends, permitting complete circularization of both strands.

In various embodiments, overhang(s) are generated in a template nucleic acid, adapter, or both template nucleic acid and adapter. Exemplary overhang length includes about or at least about 1, 2, 3, 4, 5, 6, 7, 8, 9, or 10 nucleotides. In cases where a nucleotide gap is formed 211, the gap is about or at least about 1, 2, 3, 4, 5, 6, 7, 8, 9, or 10 bases long. In order to generate the discontinuous strand, in many cases, the second strand of the adapter molecule has one or fewer bases than the first strand of the adapter molecule. For example, the second strand of the adapter has 1, 2, 3, 4, 5, 6, 7, 8, 9 or 10 few bases than the first strand of the adapter molecule. An additional feature that aids gap formation is that the second strand of the adapter lacks a 5′ phosphate. An additional feature of the adapter shown in FIG. 2 is that the second strand (located beneath the first strand) comprises phosphorothioated phosphate bonds at its 5′ end to prevent exonuclease digestion. In some cases, the first 1, 2, 3, 4, 5, 6, 7, 8, 9, or 10 phosphate bonds at the 5′ end of one strand of a double-stranded adapter are phosphorothioated.

For sticky end ligation, small adapter nucleic acid sequences are added to both ends of target nucleic acids to generate sticky ends. Small adapter nucleic acid sequence addition can be conducted during nucleic acid synthesis methods or by amplification of nucleic acids with non-canonical base (e.g., uracil) containing primers, followed by treatment of the amplification products with a mixture of nicking and nucleotide removal enzymes (e.g., UDG and EndoVIII). Exemplary overhang lengths include 4 to 12 bases. In some cases, overhangs are designed so that upon self-ligation, only one of the two strands anneals to a continuous strand and the other strand would not anneal and comprise a gap. Exemplary gap lengths include 1, 2, 3, 4, 5 and more than 5 bases.

For blunt end ligation, target nucleic acids are amplified by PCR with a first primer that has a 5′ phosphate and a second primer that lacks a 5′ phosphate. In such cases, the initial 5′ bases (e.g., 1, 2, 3, 4, 5, or more) of the second primer include phosphorothioated bonds. The PCR are self-ligated to generate a continuous circularized strand base paired to a discontinuous strand having a nick.

With respect enzymatic cleavage 202, selective removal of bases is accomplished by the incorporation of a non-canonical base pair in an extender sequence flanking a target nucleic acid. The non-canonical base pair is recognized in an enzymatic reaction that can be used to selectively remove bases from the 5′ or 3′ end of the non-canonical base pair to generate an overhang. Non-limiting examples of non-canonical bases for inclusion in adapter sequence extending from the target sequence include uracil, 3-meA (3-methyladenine), hypoxanthine, 8-oxoG (7,8-dihydro-8-oxoguanine), FapyG, FapyA, Tg (thymine glycol), hoU (hydroxyuracil), hmU (hydroxymethyluracil), fU (formyluracil), hoC (hydroxycytosine), fC (formylcytosine), 5-meC (5-methylcytosine), 6-meG (O6-methylguanine), 7-meG (N7-methylguanine), εC (ethenocytosine), 5-caC (5-carboxylcytosine), 2-hA, EA (ethenoadenine), 5-fU (5-fluorouracil), 3-meG (3-methylguanine), and isodialuric acid.

In some cases, a non-canonical base pair is recognized by one or more DNA repair enzymes, for example an enzyme that catalyzes a first step in base excision such as a DNA glycosylase. Non-limiting examples of DNA glycosylases include uracil DNA glycosylases (UDGs), helix-hairpin-helix (HhH) glycosylases, 3-methyl-purine glycosylase (MPG) and endonuclease VIII-like (NEIL) glycosylases. Examples of UDGs include, without limitation, thermophilic uracil DNA glycosylases, uracil-N glycosylases (UNGs), mismatch-specific uracil DNA glycosylases (MUGs) and single-strand specific monofunctional uracil DNA glycosylases (SMUGs). In some cases, a non-canonical base is released from an extender sequence flanking a target nucleic acid by a DNA glycosylase resulting in an abasic site. In some cases, the abasic site is further processed by an endonuclease which cleaves the phosphate backbone at the abasic site. Non-limiting examples of endonucleases include E. coli exonuclease III, S. pneumoniae and B. subtilis exonuclease A, mammalian AP endonuclease 1 (API), Drosophila recombination repair protein 1, Arabidopsis thaliana apurinic endonuclease-redox protein, Dictyostelium DNA-(apurinic or apyrimidinic site) lyase, bacterial endonuclease IV, fungal and Caenorhabditis elegans apurinic endonuclease APN1, Dictyostelium endonuclease 4 homolog, Archaeal probable endonuclease 4 homologs, mimivirus putative endonuclease 4, endonuclease IV, RecBCD endonuclease, T7 endonuclease, endonuclease II, Neurospora endonuclease, S1 endonuclease, P1 endonuclease, Mung bean nuclease I, Ustilago nuclease. In some embodiments, an endonuclease functions as both a glycosylase and an AP-lyase. In some cases, the endonuclease is endonuclease VIII, S1 endonuclease, endonuclease III, or endonuclease IV.

Returning to the workflow of FIG. 1, after the heterogeneous population nucleic acids is circularized, a partitioning occurs 108. In this first illustration, the circularized nucleic acids are partitioning into separate fractions at a concentration of about 1 circularized nucleic acid per fraction. In various embodiments, a single nucleic acid molecule includes an average of about 0.1 to about 100 molecules per fraction. Prior to performing an RCA reaction, the circularized nucleic acids are subjected to heat denaturing (e.g., about 94° C. to about 100° C. for about 3 to about 10 minutes), following by a period of cooling down (e.g., in an ice bath for about 2 to about 15 minutes). Heat denaturing of circularized nucleic acids is applicable to other methods disclosed herein.

In cases where the circularized nucleic acid does not comprise a nick or gap, the RCA reaction 109 includes a primer which is random or specific. In cases, one or a set of random primers are used to amplify a homogeneous population of circularized DNA strand. In some cases, the primer(s) comprise about or less than 30, 29, 28, 27, 26, 25, 24, 23, 22, 21, 20, 19, 18, 17, 16, 15, 14, 13, 12, 11, 10, 9, 8, 7, 6, 5, 4, or 3 bases. In some cases, the primer comprises 6 bases and is a random primer. In cases where the circularized nucleic acid does comprises a nick or gap, the continuous, circularized DNA strands serve as a template for the amplification reaction.

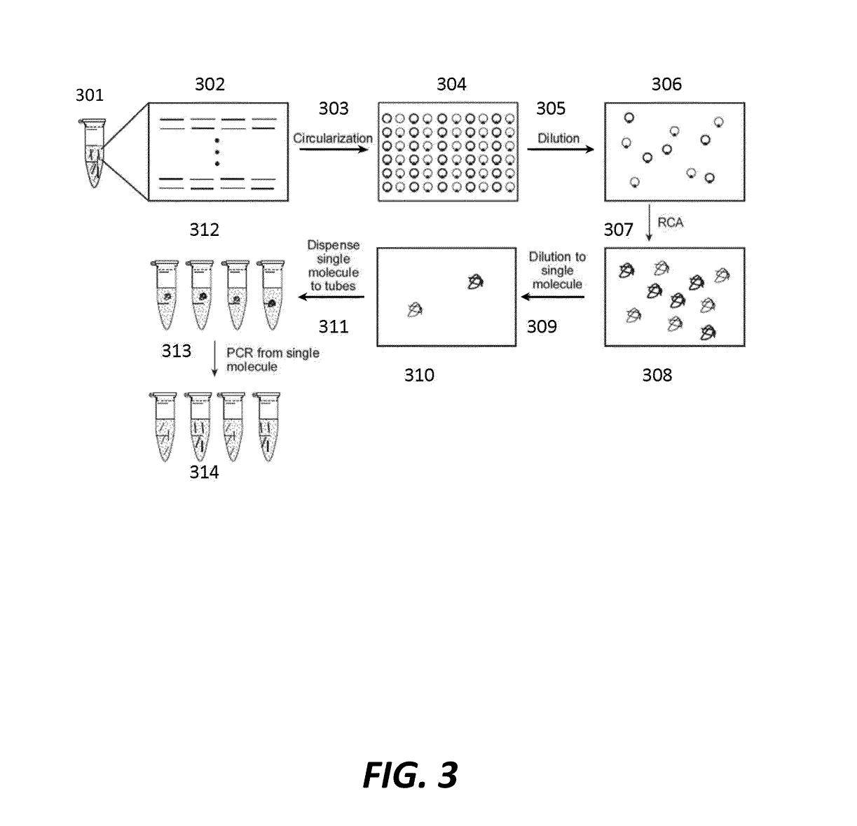

A second example procedure for cell-free sorting and cloning is depicted in FIG. 3. As with the example in FIG. 1, a starting sample 301 includes a heterogeneous population of double-stranded target nucleic acids 302. The heterogeneous population of double-stranded target nucleic acids is circularized 304 and subject to a first dilution 305 to generate a pool 306. The various techniques previously described for circularization are applicable in this example as well. However, unlike in the first method, the heterogeneous population of circularized nucleic acids is not partitioned down to roughly single molecule fractions at this stage. Instead, dilution of circularized nucleic acids is about or less than about 100 nM, 10 nM, 1 nM, 100 pM, 10 pM, 1 pM, 100 fM, 10 fM, or 5 fM. As in the example in FIG. 1, the heterogeneous population is optionally heat denatured at this point. A RCA reaction 307 of the mixture is performed and the population is subject to second dilution 309 and the second diluted pool 310 is dispensed 311 into tubes 312 with an average of 1 single amplicon per tube. PCR 313 from the single molecule results in an amplified clonal population 314. In some cases, sequencing of amplification product occurs after the RCA reaction 307. In some cases, sequencing of amplification product occurs after the PCR step 3131. Sequencing data corresponding to clonal populations is compared to that of predetermined sequence(s).

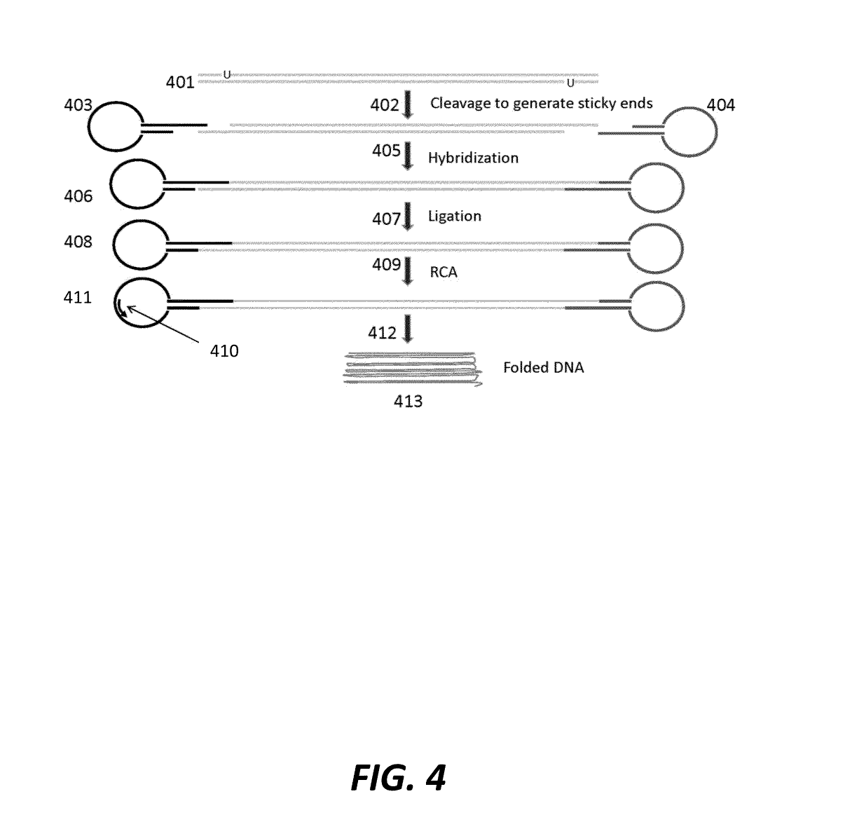

A third example cell-free sorting and cloning procedure incorporating hairpins is depicted in FIG. 4. As with the first example in FIG. 1, starting sample includes a heterogeneous population of double-stranded target nucleic acids 401. In this case, a double-stranded nucleic acid 401 comprises a uracil base near the 5′ end of the first strand and a uracil base near the 5′ end of the second strand. In some cases, uracil bases are incorporated into the population of nucleic acids to be sorted using primers comprising one or more uracil bases. In other instances, uracil is incarnated into a nucleic acid by chemical synthesis. Depending on the desired overhang, a uracil base is incorporated near the 5′ or 3′ end of a strand such that it is located about 1, 2, 3, 4, 5, 6, 7, 8, 9, 10, 11, 12, 13, 14, 15, 16, 17, 18, 19, 20 or more bases from the end of the strand.

As previously mentioned, to generate sticky ends of the double-stranded nucleic acid, cleavage 402 occurs in the presences nicking and nucleotide removal enzymes (e.g., UDG and EndoVIII). Each end of the duplex is a set of DNA hairpins 403, 404 having different sequences, the components hybridize 405 and become in close association with each other 406. The hybridized components are then mixed with ligation reagents and subject to a ligation reaction 407. The ligation product 408 is a single-stranded circularized DNA that comprises a region of self-hybridization that prevents entanglement of and hybridization between two DNA molecules. The single-stranded nucleic acids are amplified by RCA 409, in the presence of a primer 410, where the amplification product 411 folds 412 into compact nanoballs 412. In some cases, sequencing of amplification product occurs after the RCA reaction 409. In some cases, sequencing of amplification product occurs after a second amplification, e.g., PCR. Sequencing data corresponding to clonal populations is compared to that of predetermined sequence(s).

In some cases, the single-stranded nucleic acids are heat denatured and subject to a first dilution prior to RCA. In some cases, the RCA reaction product is partitioned into single molecule fractions, i.e., a second dilution. RCA products are optionally further amplified, for example by PCR to generate fractions having clonal copies of the single parent molecule. A benefit of generating single-stranded circular DNA with areas of self-complementarity is that amplification products, e.g., RCA products, are more dispensed into single molecule fractions.

As in the procedure illustrated in FIG. 3, for the first dilution of circularized nucleic acids, a concentration of about or less than about 100 nM, 10 nM, 1 nM, 100 pM, 10 pM, 1 pM, 100 fM, 10 fM, or 5 fM is used. In some cases, the circularized nucleic acid is diluted to a concentration of about 1 pM.

In some embodiments, a double-stranded target nucleic acid within a sample to be sorted is circularized by ligation to two DNA hairpins. In some cases, the two DNA hairpins comprise the same nucleic acid sequence. In some cases, the two DNA hairpins comprise a different nucleic acid sequence. In some cases, a DNA hairpin incorporated in a circularized target nucleic acid comprises between about 20 bases and about 150 bases. In some cases, a DNA hairpin comprises about 30, 35, 40, 45, 50, 55 or 60 bases. In some cases, a stem of a DNA hairpin comprises between about 5 and about 20. In some cases, a stem of a DNA hairpin comprises about 5, 6, 7, 8, 9, or 10 base pairs. In some cases, a loop of a DNA hairpin comprises between about 15 and about 100. In some cases, a loop of a DNA hairpin comprises about 20, 30, 40, 50, 60, 70, 80, 90 or 100 bases.

In some embodiments, a double-stranded target nucleic acid within a sample to be sorted is circularized by self-ligation. In some embodiments, a target nucleic acid is prepared for circularization by self-ligation by a method comprising the addition of a small adapter nucleic acid sequence to one or both ends of the target nucleic acid. In some cases, for a target nucleic acid comprising small adapter nucleic acid sequences at both ends, a first small adapter nucleic acid sequence is added to a first end of the target nucleic acid and a second small adapter nucleic acid sequence is added to a second end of the target nucleic acid. In some cases, the first small adapter nucleic acid sequence comprises a nucleic acid sequence that is the same or complementary to a nucleic acid sequence of the second small adapter nucleic acid sequence. In some cases, the first small adapter nucleic acid sequence comprises a nucleic acid sequence that is different or not complementary to a nucleic acid sequence of the second small adapter nucleic acid sequence.

In one aspect of the nucleic acid sorting methods described herein, target nucleic acids are subject to partitioning into one or more fractions. In various embodiments, the target nucleic acids are circularized. In some embodiments, the target nucleic acids are amplified prior to partitioning. In some embodiments, the target nucleic acids are partitioned prior to amplification. In some embodiments, the target nucleic acids are partitioned prior to and after amplification. In some cases, wherein the target nucleic acids are partitioned into fractions prior to amplification, the target nucleic acid(s) within each fraction serve as template(s) or parent nucleic acid(s) for the amplification reaction. Therefore, the amplification products, or amplicons, are clonal copies of the parent nucleic acid(s) within each fraction. In some embodiments, partitioning comprises diluting the target nucleic acids, and/or amplicons thereof, in a solution, so that an aliquot of the diluted solution comprises a calculated or estimated number of nucleic acid molecules. In some embodiments, the concentration of nucleic acids within a solution of target nucleic acids and/or amplicons thereof, either diluted or non-diluted, is measured. The solution is then partitioned (e.g., aliquoted) into two or more fractions so that each fraction comprises, on average, a calculated number of nucleic acid molecules (e.g., target nucleic acids and/or amplicons thereof). In some embodiments, dilution comprises diluting a solution of target nucleic acids and/or amplicons to a DNA concentration that is about or less than about 100 nM, 10 nM, 1 nM, 100 pM, 10 pM, 1 pM, 100 fM, 10 fM, or 5 fM. In some embodiments, partitioning is performed without dilution, for example, by aliquoting small enough volumes so that each fraction has, on average, a small number of nucleic acid molecules (e.g., a single molecule).

In some embodiments, a solution comprising a sample of target nucleic acids and/or amplicons thereof, is partitioned into about or at least about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 or more fractions. In some embodiments, the solution is partitioned by aliquoting volumes of the solution into fractions, wherein the volume of one or more of the aliquots is from about 1 pl to about 1 ul. In some embodiments, a solution is partitioned into volumes of about or less than about 100 ul, 90 ul, 80 ul, 70 ul, 60 ul, 50 ul, 40 ul, 30 ul, 20 ul, 15 ul, 10 ul, 9 ul, 8 ul, 7 ul, 6 ul, 5 ul, 4 ul, 3 ul, 2 ul, 1.5 ul, 1 ul, 0.9 ul, 0.8 ul, 0.7 ul, 0.6 ul, 0.5 ul, 0.4 ul, 0.3 ul, 0.2 ul, 0.1 ul, 90 nl, 80 nl, 70 nl, 60 nl, 50 nl, 40 nl, 30 nl, 20 nl, 10 nl, 9 nl, 8 nl, 7 nl, 6 nl, 5 nl, 4 nl, 3 nl, 2 nl, 1 nl, 0.9 nl, 0.8 nl, 0.7 nl, 0.6 nl, 0.5 nl, 0.4 nl, 0.3 nl, 0.2 nl, 0.1 nl, 90 pl, 80 pl, 70 pl, 60 pl, 50 pl, 40 pl, 30 pl, 20 pl, 10 pl, 5 pl or less.

In some embodiments, a solution is partitioned such that, on average, each fraction comprises about or at least about 0.001 to 200, 0.1 to 2, or 0.5 to 10 nucleic acid molecules. In some cases, one or more fractions do not comprise a nucleic acid molecule. In some cases, one or more fractions comprise one nucleic acid molecule. In some cases, one or more fractions comprise two or more nucleic acid molecules. In embodiments, a nucleic acid molecule includes, but is not limited to, a target nucleic acid molecule (e.g., circularized), an amplification product of a target nucleic acid molecule (e.g., RCA amplicon or concatemer), or both. In some embodiments, a solution is partitioned so that each fraction comprises, on average, a single nucleic acid molecule. In some embodiments, a solution is partitioned so that, on average, each fraction comprises less than about 100, 90, 80, 70, 60, 50, 40, 30, 25, 20, 19, 18, 17, 16, 15, 14, 13, 12, 11, 10, 9.5, 9, 8, 5, 8, 7.5, 7, 6.5, 6, 5.5, 5, 4.5, 4, 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 0.08, 0.07, 0.06, 0.05 or less nucleic acid molecules.

In some embodiments, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 91, 92, 93, 94, 95, 96, 97, 98, 99, 99.5% or more of the partitioned fractions comprise a nucleic acid. In some embodiments, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 91, 92, 93, 94, 95, 96, 97, 98, 99, 99.5% or more of the partitioned fractions comprise a single nucleic acid. In some instances, a sample is partitioned into single molecule (e.g., on average, 0.1 to 2) fractions and the fractions are amplified. In such cases, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 91, 92, 93, 94, 95, 96, 97, 98, 99, 99.5% or more of the fractions comprise amplicons from one target parent nucleic acid. In some cases, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 91, 92, 93, 94, 95, 96, 97, 98, 99, 99.5% or more of the fractions comprise amplicons from two or more target parent nucleic acids. In some cases, about 1, 2, 3, 4, 5, 6, 7, 8, 9, 10, 11, 12, 13, 14, 15, 16, 17, 18, 19, 20, 25, 30, 35, 40, 45, 50% or more of the fractions do not comprise amplicons.

In some embodiments, at least one or more partitioned fractions comprise two or more nucleic acid molecules, wherein at least two of the nucleic acid molecules have the same nucleic acid sequence. In some embodiments, at least one or more partitioned fractions comprise two or more nucleic acid molecules, wherein at least one of the nucleic molecules has a different nucleic acid sequences from another nucleic acid molecule in the same fraction. In some cases, fractions comprise, on average about or less than about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10 different nucleic acid molecules per fraction, wherein the nucleic acids molecules include target nucleic acids and/or amplicons thereof.

In some embodiments, a sample comprising a plurality of target nucleic acids is partitioned prior to amplification. In such cases, the sample is optionally partitioned into fractions with one or more additional reagents, e.g., amplification reaction reagents. In some embodiments, a sample comprising a plurality of target nucleic acids is partitioned after the target nucleic acids are amplified, and therefore the sample comprises both the target (parent) nucleic acids and amplicons thereof. In some cases, a solution comprising target nucleic acids and amplicons thereof is partitioned into fractions comprising, on average, about or at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10 or more nucleic acid molecules. In some cases, a fraction comprises a target nucleic acid molecule(s). In some cases, a fraction comprises an amplicon(s). In some cases, a fraction does not comprise a nucleic acid molecule.

In some embodiments, a target nucleic acid is amplified prior to and/or after partitioning and the amplification product comprises a plurality of copies of the target (parent) nucleic acid packaged together, for example, by covalent bonds and/or adherence to a common binding partner, such as a bead. In some cases, each package comprises, on average, about or at least about 2, 3, 4, 5, 6, 7, 8, 9, 10, 11, 12, 13, 14, 15, 20, 25, 30, 40, 50, 60, 70, 80, 90, 100, 150, 200, 300, 400, 500, 600, 700, 800, 900, 1000 or more copies of a parent nucleic acid. In some embodiments, a solution comprising packages of copies are partitioned into two or more fractions such that, on average, each fraction comprises about or less than about 100, 90, 80, 70, 60, 50, 40, 30, 25, 20, 19, 18, 17, 16, 15, 14, 13, 12, 11, 10, 9.5, 9, 8, 5, 8, 7.5, 7, 6.5, 6, 5.5, 5, 4.5, 4, 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.09, 008, 0.07, 0.06, 0.05, 0.04, 0.03, 0.02, or 0.01 packages. In some embodiments, a package comprises a concatemer. In some embodiments, a package forms a nanoball. In some cases, a nanoball is about or at least about 20 nm, 50 nm, 100 nm, 500 nm, 1 um, 2 um, 3 um, 4 um, 5 um or larger in diameter. In some cases, a nanoball is from about 20 nm to about 5 um, from about 20 nm to about 4 um, from about 20 nm to about 3 um, from about 20 nm to about 2 um, from about 20 nm to about 1 um, or from about 20 nm to about 500 nm in diameter.

In some embodiments, nanoballs comprising copies of a parent nucleic acid are contacted to/captured by a patterned surface during partitioning. In some embodiments, the pattern surface comprises features that are design to allow for the capture of not more than one nanoball per feature. In some embodiments, the features of a patterned surface are sized such that only one nanoball can fit either in or on a feature. In some embodiments, captured nanoballs on a surface are transferred to a nanowell chip. In some cases, the feature of a surface has a cross-section of about or at least about 20 nm, 50 nm, 100 nm, 500 nm, 1 um, 2 um or larger. In some cases, the feature of a substrate has a cross-section of about or less than about 2 um, 1 um, 900 nm, 800 nm, 700 nm, 600 nm, 500 nm, 400 nm, 300 nm, 200 nm, 150 nm, 100 nm, 80 nm, 60 nm, 40 nm or 20 nm.

In some instances, a surface is patterned with a functionalized active and/or passive area(s). In such cases, active areas are able to bind to an amplification product and passive areas are inefficient or incapable of binding to an amplification product. For example, in some cases, an active area comprises a coating with an amine-terminated moiety as described in surface/substrate modification sections provided elsewhere herein. An exemplary class of amine-terminated moiety molecules includes amino silanes. As another example, in some cases, a passive area comprises a coating with a fluorinated moiety as described in the surface/substrate modification sections provided elsewhere herein. As another example, a passive area comprises a coating with a fluorinated surface. In some instances, in a microwell or nanowell context, areas of functionalization are located within the well. In some cases, the amplification product is a nanoball. In other cases, the amplification product is not a nanoball.

In some embodiments, active areas of a surface are separated by about or at least about 20 nm, 50 nm, 100 nm, 500 nm, 1 um, 2 um, 50 um, 500 um or more. In some cases, active areas of a surface are separated by a distance less than about 2 mm, 1 mm, 500 um, 100 um, 50 um, 10 um, 5 um, 4 um, 3 um, 2 um, 1 um, 500 nm, 100 nm, 50 nm or 20 nm. In some embodiments, methods for active and passive functionalization of surfaces described elsewhere herein in relation to oligonucleic acid synthesis are functionalize substrates used for partitioning. In addition, in some embodiments, substrates described elsewhere herein for oligonucleic acid synthesis also maintain/capture partitioned fractions using nucleic acid sorting. For example, in some cases, a substrate comprising one or more wells, and optionally a plurality of nanowells with each well, is holds partitioned fractions of a nucleic acid population.

In some embodiments, nucleic acids are partitioned into fractions using droplets, emulsions, pores of a gel, beads, features of a microfluidic device, addressable spots of a substrate, nanowells, or any partitioning options known in the art. In some embodiments, fractions comprise droplets in an emulsion. In some cases, a population of droplets is formed so that, on average, there are about or at least about 0.1 to 10 or more nucleic acid molecules (e.g., target nucleic acids and/or amplicons thereof) within a droplet. In some embodiments, a droplet further comprises or is supplemented with one or more reagents for performing an amplification reaction, e.g., primer(s), polymerase, dNTPs, buffers, nucleic acid dye, or combination thereof. In one example, an emulsion of droplets is subjected to amplification reaction conditions and the droplets are sorted, for example, by flow cytometry. In droplets starting off with one parent nucleic acid molecule, the amplification products in each droplet are copies from the same parent, allowing for cell-free sorting. In another example, emulsion amplification is performed on beads. In some cases, an emulsion comprises a plurality of beads and each bead comprises, on average, about or at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, or more target nucleic acid molecules so that after amplification, each bead comprises clonally amplified nucleic acid molecules. In some cases, a droplet comprises, on average, 0.1 to 10 beads.

In some embodiments, a heterogeneous population of target nucleic acids is partitioned into nanowells. In some cases, the target nucleic acids are circularized target nucleic acids, wherein the target nucleic acids are circularized prior to, or after partitioning into nanowells. In some embodiments, amplification products of a heterogeneous population of target nucleic acids are partitioned into nanowells. In some cases, target nucleic acids are amplified prior to and/or after partitioning into nanowells. In some cases, the amplification products are RCA products. In some cases, the nucleic acids partitioned into fractions of nanowells are amplified within the nanowells. In some cases, the amplification is RCA. In some cases, the amplification is PCR. In some cases, each fraction in a nanowell comprises a dilute sample of nucleic acids. In some cases, each fraction comprises, on average, a single molecule of nucleic acid. In some cases, each fraction comprises, on average, about or less than about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, or 5 nucleic acid molecules. In some cases, each fraction comprises, on average, about or less than about 0.1 to 10, 0.5 to 2.0, or 0.3 to 1.50 nucleic acid molecules. In some embodiments, any step of a cell-free sorting method provided herein is performed within one or more nanowells. In some embodiments, the nanowells are a plurality of nanowells of a substrate described herein. In some cases, nucleic acids are partitioned into nanowells of a substrate, wherein one or more of the nanowells have a diameter between about 0.2 mm and about 10 mm, between about 0.2 mm and about 5 mm, between about 0.2 mm and about 2 mm, between about 0.5 mm and about 10 mm, between about 0.5 mm and about 5 mm, or between about 0.5 mm and about 2 mm. In some embodiments, a diameter of a nanowell is about 0.5, 0.6, 0.7, 0.8, 0.9, 1.0, 1.1, 1.2, 1.3, 1.4, 1.5, 1.6, 1.7, 1.8, 1.9, or 2 mm in diameter. In some cases, a nanowell has an internal depth of between about 0.1 mm and about 5 mm, between about 0.1 mm and about 4 mm, between about 0.1 mm and about 3 mm, between about 0.1 mm and about 2 mm, or between about 0.1 mm and about 1 mm. In some embodiments, a nanowell has an internal depth of about 0.1, 0.2, 0.3, 0.4, 0.5, 0.6, 0.7, 0.8, 0.9, or 1 mm. In some cases, the interior of a nanowell has a capacity to hold a volume less than about 10, 9, 8, 7, 6, 5, 4, 3, 2, 1, 0.9, 0.8, 0.7, 0.6, 0.5, 0.4, 0.3, 0.2, or 0.1 ul. In some embodiments, the interior of a nanowell has a capacity to hold a volume between about 0.1 ul and about 10 ul, between about 0.1 ul and about 4 ul, between about 0.1 ul and about 2 ul, between about 0.1 ul and about 1 ul, or between about 0.1 ul and about 0.5 ul. In some embodiments, the interior of a nanowell has a capacity to hold a volume of about 0.1, 0.2, 0.3, 0.4, 0.5, 0.6, 0.7, 0.8, 0.9, or 1 ul.

In some embodiments, amplification includes the addition of labeled or tagged primers. Exemplary forms of labeling include, without limitation, a fluorescent label, a chemiluminescent label, a quencher, a radioactive label, biotin, and gold, or combinations thereof. In some cases, tagged primers are included wherein amplification is performed on beads. In such cases, beads comprising amplicons may be screened using the tag, e.g., biotinylated amplicons are screen with streptavidin. In some cases, beads comprising amplicons are dispensed onto a nanowell plate. In some cases, beads are dispensed so that, on average, each nanowell comprises, on average, about or at least about 0.1, 0.2, 0.3, 0.4, 0.5, 0.6, 0.7, 0.8, 0.9, 1, 1.1, 1.2, 1.3, 1.4, 1.5, 1.6, 2, 3, 4, 5, or more beads. In some cases, each nanowell comprises, on average, at most about 5, 4, 3, 2, 1.6, 1.5, 1.4, 1.3, 1.2, 1.1, 1, 0.5, 0.4, 0.3, 0.2, 0.1 or fewer beads. In some embodiments, the nucleic acids attached to the plated beads are subjected to another round of amplification, e.g., by PCR.

In one aspect of nucleic acid sorting methods described herein, amplicons of target nucleic acids are amplified in a second amplification reaction. In some embodiments, target nucleic acids are amplified in a first amplification reaction, the target nucleic acids and amplicons thereof are partitioned into two or more fractions, and at least one of the two or more fractions are subjected to the second amplification reaction. In some embodiments, target nucleic acids are partitioned into two or more fractions, the target nucleic acids are amplified in a first amplification reaction within the fractions, and then the target nucleic acids and amplification products thereof are subjected to the second amplification reaction. In some embodiments, the target nucleic acids are circularized. In some embodiments, the second amplification reaction comprises one or more amplification steps. In some embodiments, one of the amplification steps comprises polymerase chain reaction (PCR). In some embodiments, one of the amplification steps comprises multiple displacement amplification (MDA). In some embodiments, any round of amplification described herein (e.g., first, second, or any subsequent reaction) provides at least about a 5, 10, 50, 100, 500, 1000, 5000, 10000, 50000, 100000, 500000, 1000000, 5000000, 10000000, 100000000, or 1000000000 fold amplification of a parent nucleic acid.

In some cases, an amplicon of RCA comprises a plurality of copies of the target nucleic acid packaged together in a concatemer. In some cases, an amplicon of a RCA reaction refers to a concatemer. For example, reference to a single molecule of a RCA product, e.g., single amplicon or single molecule, is inclusive of a concatemer comprising a plurality of copies of a target nucleic acid sequence. In some cases, a package comprises covalently linked copies of a target sequence, e.g., a concatemer. In some cases, a concatemer comprises about or at least about 2, 3, 4, 5, 6, 7, 8, 9, 10, 11, 12, 13, 14, 15, 16, 17, 18, 19, 20, 25, 30, 40, 50, 60, 70, 80, 90, 100, 120, 140, 150, 160, 180, 200, 150, 300, 400, 500, 600, 700, 800, 900, 1000 or more copies of a target sequence.

In various embodiments, the methods described herein for DNA amplification include a DNA polymerase with 3′ to 5′ and/or 5′ to 3′ exonuclease activity. In some embodiments, amplification methods described herein include the addition of high-fidelity wild-type polymerases or engineered enzymes, such as high fidelity B-family polymerases, Pyrococcus furiosus DNA Polymerase iProof Hi-fidelity DNA Polymerase (Bio-Rad), Pfu DNA polymerase (Promega), KAPA HiFi DNA Polymerase (KAPA Biosystems), Phusion High-Fidelity DNA Polymerase (New England Biolabs), Q5 High-Fidelity DNA Polymerase (New England BioLabs), AccuPrime Pfx (Life Technologies), PfuUltra II Phusion HS (Agilent), PfuUltra High-Fidelity DNA Polymerase (Agilent), Platinum Taw HiFi (Life Technologies), and KOD DNA Polymerase (EMD). In some cases, an enzyme used in an amplification reaction has an error rate of less than 1 in 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 45, 50, 55, 60, 65, 70, 75, 80, 85, 90, 95, 100, 110, 125, 150, 200, 250, 300, 400, 500, 750, 1000, 2000, 3000, 4000, 5000, 10000, 15000, 20000 bases. Enzymes or enzyme blends that are suitable for long range PCR, for example, for the amplification of fragments that are longer than 2, 3, 4, 5, 6, 7, 8, 9, 10, 15, 20 kilobases, or longer may also be used for amplification reactions described herein. In some cases, a hot-start amplification reaction is performed using a suitable enzyme or enzyme mixture, for example, KAPA2G Fast HotStart DNA Polymerase (KAPA Biosystems), KAPA2G Robust HotStart DNA Polymerase (KAPA Biosystems), KAPA HiFi HotStart DNA Polymerase (KAPA Biosystems), KAPA Long Range HotStart DNA Polymerase (KAPA Biosystems), Go Taq Hot Start Polymerase (Promega), Hot Start Taq DNA Polymerase (New England BioLabs), HotStarTaq DNA Polymerase (Qiagen), Maxima Hot Start Taq DNA Polymerase (Thermo Scientific), TrueStart Hot Start Taq DNA Polymerase (Thermo Scientific), Phusion Hot Start II High-Fidelity DNA Polymerase (Thermo Scientific), PfuTurbo Cx Hotstart DNA Polymerase (Agilent Technologies), Hot Start TaKaRa Taq DNA Polymerase (Clone Tech/Takara Bio).

In some embodiments, nucleic acids amplified within partitioned fractions (nucleic acid products) are starting materials for one or more additional methods. In some cases, the nucleic acid products of the fractions are sequenced. In some embodiments, the nucleic acid products of a fraction are combined with products from another fraction comprising the same population of products. In some cases, nucleic acid products are treated with an enzyme. For example, nucleic acid products comprising concatemers are treated to separate copies within the concatemers. In some cases, nucleic acid products are inserted into a vector. In some cases, nucleic acid products are cloned. In some cases, nucleic acid products are expressed in vivo. In some cases, nucleic acid products are expressed in vitro.

In one aspect of the nucleic acid sorting methods described herein, one or more partitioned fractions comprise a parent nucleic acid molecule and clonal amplification products thereof. In some embodiments, the methods further comprise sequencing one or more partitioned fractions to identify fractions comprising a homogeneous population of nucleic acids. In some embodiments, sequence variation within a fraction is less than about 1 in 5, 6, 7, 8, 9, 10, 15, 20, 25, 30, 35, 40, 45, 50, 60, 70, 80, 90, 100, 120, 140, 160, 180, 200, 250, 300, 400, 500 bases or less. In some cases, sequence variation within a fraction is limited by the error rate of an enzyme used to generate the amplification products within the fraction, e.g., the polymerase.

In some embodiments, methods for cell-sorting described herein include hybridizing a discontinuous strand of circularized DNA having 1, 2, 3, 4, 5, 6, 7, 8, 9, 10, 11, 12, 13, 14, 15, 20 or more fewer bases than a continuous strand of the circularized DNA to which it is hybridized, generating one or more gaps, or abasic sites. In some embodiments, a double-strand adapter sequence bridges the two ends of a target sequence, and the second strand of the adapter lacks a 5′ phosphate so that it does not ligate at this end with the second strand of the target nucleic acid. In some embodiments, the gap is formed at a juncture of the second strand of the adapter and the second strand of a target nucleic acid. In some embodiments, the continuous circular strand comprises about or at least about 30, 40, 50, 60, 70, 80, 90, 100, 120, 140, 160, 180, 200, 250, 300, 350, 400, 450, 500, 550, 600, 650, 700, 800, 900, 1000, 1100, 1200, 1300, 1400, 1500, 1600, 1700, 1800, 1900, 2000 or 2500 bases.

In some embodiments, a population of target nucleic acids is diluted prior to RCA. For example, the population of target nucleic acids is diluted to a DNA concentration of about or less than about 100 nM, 10 nM, 1 nM, 100 pM, 10 pM, 1 pM, 100 fM, 10 fM, 5 fM, or less prior to RCA reaction. In some embodiments, the amplicons are diluted prior to partitioning so that a given volume would comprise from about 0.1 to about 2 amplicons. In some embodiments, the given volume is the volume of amplicons partitioned into a fraction. In some cases, the given volume is less than or about 100 ul, 50 ul, 20 ul, 10 ul, 9 ul, 8 ul, 7 ul, 6 ul, 5 ul, 4 ul, 3 ul, 2 ul, 1 ul, 0.9 ul, 0.8 ul, 0.7 ul, 0.6 ul, 0.5 ul, 0.4 ul, 0.3 ul, 0.2 ul, 0.1 ul, 90 nl, 80 nl, 70 nl, 60 nl, 50 nl, 40 nl, 30 nl, 20 nl, 10 nl, 1 nl, 50 pl, 10 pl or 1 pl. In some embodiments, the partitioned volume is between about 10 pl and 1 ul, including any volumes within the provided ranges. In some embodiments, the sample of amplicons is diluted about or at least about 10, 100, 1000 fold or more prior to partitioning. In various aspects of the methods, in order to partition a sample of amplicons into fractions having, on average, about 0.1 to about 2 amplicons per fraction, the concentration of the sample of amplicons is measured prior to partitioning. In some embodiments, the sample is partitioned into fractions having, on average, 0.001 to 200, 0.1 to 2, 0.5 to 2.0, 0.1 to 20, 0.5 to 1.3, or 0.1 to 1 DNA molecules or amplicons per fraction. In some cases, one or more fractions will not comprise an amplicon. In some cases, one or more fractions will comprise one amplicon. In some cases, one or more fractions will comprise two or more amplicons. In some embodiments, the amplicons are single-stranded.

In some embodiments, an amplification product is partitioned into about or at least about 2, 3, 4, 5, 6, 7, 8, 9, 10, 15, 20, 30, 40, 50, 60, 70, 80, 90, 100 or more fractions. In some embodiments, the sample is partitioned into from about 2 fractions to about 100 fractions. In various embodiments, a sample is partitioned into two or more sets of fractions, where one set of fractions comprises, on average, a first number of amplicons per fraction, and another set of fractions comprises, on average, a second number of amplicons per fraction. For example, a first number of amplicons is from about 0.1 to about 2 amplicons per fraction. As another example, a second number of amplicons is from about 1 amplicon to about 10 amplicons per fraction.

In some embodiments, the target nucleic acids are prepared for hybridization and ligation to an adapter molecule by the formation of sticky ends or overhangs at one or both ends of the target nucleic acids. In some cases, the overhang is a 3′ overhang. In some cases, the overhang is a 5′ overhang. In some cases, the target nucleic acid has both a 3′ and a 5′ overhang. In some cases, an overhang of a 3′ and/or 5′ strand of a double-stranded target nucleic acid is 3, 4, 5, 6, 7, 8, 9, 10, 11, 12, 13, 14 or 15 bases long. In some embodiments, the adapter comprises one or two sticky ends or overhangs. In some cases, the adapter overhang is a 3′ overhang. In some cases, the adapter overhang is a 5′ overhang. In some cases, the adapter has both a 3′ and a 5′ overhang. In some embodiments, a 3′ and/or 5′ overhang of an adapter is 3, 4, 5, 6, 7, 8, 9, 10, 11, 12, 13, 14 or 15 bases in length. In some embodiments, circularization of the target nucleic acids is performed using a ligase. Examples of suitable ligases include, but are not limited to, T4 DNA ligase, T3 DNA ligase, T7 DNA ligase, Taq DNA ligase, Ampligase, 7N DNA ligase, and RNA ligase. In some embodiments, circularization of the target nucleic acids is performed using a polymerase.

In another aspect of the disclosure, provided are methods for purifying a sample comprising a heterogeneous population of target nucleic acids. In various embodiments, the sample comprises a plurality of synthesized nucleic acids (including synthesized, assembled nucleic acids). In various aspects, provided are methods for purifying a sample of target nucleic acids having at least two different nucleic acid sequences, the methods comprising partitioning (e.g., by aliquoting) the sample into partitions of packages of nucleic acids such that each partition receives on average from about 0.001 to about 2 packages, wherein each package of nucleic acids comprises nucleic acids from a single one of the at least two different nucleic acid sequences. In some embodiments, the target nucleic acids are amplicons. In some embodiments, the sample comprises about or at least about 2, 3, 4, 5, 6, 7, 8, 9, 10, 20, 30, 40, 50, 60, 70, 80, 90 or 100 nucleic acids with different nucleic acid sequences. In some embodiments, the number of packages is about or at least about 2, 3, 4, 5, 6, 7, 8, 9, 10, 20, 30, 40, 50, 60, 70, 80, 90 or 100.

In some embodiments, the sample is partitioned into droplets, beads, wells, resolved features of a substrate, discrete volumes in a gel, or a combination thereof. In some embodiments, the partition comprises droplets in an emulsion and wherein the droplets in the emulsion are sorted. In some embodiments, the droplets in the emulsion are sorted by flow cytometry. In some cases, the substrate comprises a pattern surface comprising active and passive areas (e.g., substrates described elsewhere herein), wherein the active areas are capable of retaining the packages and the passive areas are not capable of retaining the packages. In some embodiments, an active area of the structure is capable of holding at most one package.

In some embodiments, a method for purifying a sample of target nucleic acids further comprises performing nucleic acid amplification reactions within the partitions. In some cases, the nucleic acid amplification comprises PCR. In some cases, the nucleic acid amplification comprises MDA. In some embodiments, the partition comprises the package of nucleic acids and one or more reagents for performing an amplification reaction. For example, the partition comprises one or a set of primers. As another example, the partition comprises a DNA polymerase. In a further example, the partition comprises a nucleic acid dye. In some cases, the nucleic acid dye comprises N′,N-dimethyl-N-[4-[(E)-(3-methyl-1,3-benzothiazol-2-ylidene)methyl]-1-phenylquinolin-1-ium-2-yl]-N-propylpropane-1,3-diamine.

In some cases, methods disclosed herein for isolations, sequencing, and subsequent selection of a single clone in a heterogeneous population of nucleic acid sequences provides an efficient procedure for generating an error free clone from a population of clone nucleic acids containing an error. In some embodiments, a heterogeneous population of nucleic acids comprises oligonucleic acid synthesis products (including assembled products thereof) comprising a predetermined sequence and one or more oligonucleic acid synthesis products comprising a sequence that differs by one or more bases from the predetermined sequence. One of skill in the art would generally be aware of methods for correcting such errors once identified, such as through PCR-based point mutation error correction.

In various aspects, a cell-free method for correcting error in a sample of heterogeneous nucleic acid sequences comprises (a) providing a heterogeneous sample of target nucleic acids, wherein one or more of the nucleic acids has a different sequence from one or more of the other nucleic acids, (b) partitioning the target nucleic acids of the sample into at least two different fractions; and (c) generating isolated copies of the target nucleic acids in each of the least two or more fractions. To determine error rate, the sequence encoded by a target nucleic acid is compared to the sequence of a predetermine nucleic acid sequence. In some embodiments, one or more of the target nucleic acids comprise 250 or more bases. In some embodiments, at least 5 isolated copies of the partitioned target nucleic acids are generated per fraction. In some embodiments, the isolated copies have an error rate of less than 1 in 10,000 bases. In some embodiments, the isolated copies have an error rate of less than 1 in 15000, less than 1 in 20000, less than 1 in 25000, less than 1 in 30000, less than 1 in 40000, less than 1 in 50000, less than 1 in 60000, less than 1 in 70000, less than 1 in 80000, less than 1 in 90000, or less than 1 in 100000 bases.

In some embodiments, the heterogeneous sample comprises at least 2, 3, 4, 5, 6, 7, 8, 9, 10, 15, 20, 30, 40, 50, 100 or more nucleic acids having a sequence different from another sequence within the sample. In some embodiments, one or more of the target nucleic acids within a sample comprise about or at least about 100, 200, 300, 400, 500, 600, 700, 800, 900, 1000, 1100, 1200, 1300, 1400, 1500, 1750, 2000, 2500, 3000, 4000, or 5000 bases. In some embodiments, generating isolated copies of the different target nucleic acids comprises performing a nucleic acid amplification reaction in a diluted sample. In some embodiments, the nucleic acid amplification reaction comprises rolling cycle amplification (RCA).

In some embodiments, a cell-free method for correcting error in a sample of heterogeneous nucleic acid sequences further comprises performing a nucleic acid amplification reaction in one or more of the fractions using a DNA polymerase. In some embodiments, the isolated copies have an error rate that is about the same (e.g., about 20% lower or higher) as the maximum error rate of the DNA polymerase. In some embodiments, the isolated copies have an error rate that is about the same (e.g., about 20% lower or higher) as the average error rate of the DNA polymerase. In some embodiments, the isolated copies have an error rate that is about the same (e.g., about 20% lower or higher) as the minimum error rate of the DNA polymerase. In some embodiments, the DNA polymerase is selected from the group consisting of Q5 DNA polymerase (NEB), Kapa HiFi polymerase (Kapa), Herculase Fusion II and Pfu DNA polymerase (Agilent), and Phusion DNA polymerase (ThermoFisher).

In some embodiments, the isolated copies comprise about or at least about 2, 5, 10, 15, 20, 50, 500, 5000, or 50000 copies of each of the target nucleic acids. In some embodiments, the isolated copies have at least 0.001, 0.01, 0.1, or 1 femtomoles of each of the target nucleic acids. In some embodiments, the method further comprises sequencing nucleic acids from one or more fractions. In some embodiments, two or more of the nucleic acids within a fraction have a variation between sequences of less than 1:10, 1:100, 1:500, 1:1000, 1:2000, 1:3000, 1:4000, 1:5000, 1:6000, 1:7000, 1:8000, 1:9000, or 1:10000 bases. In some embodiments, two or more of the target nucleic acids differ in sequence by more than 1 difference for every 5 bases.

Gene Library Generation

In a further aspect of the disclosure, provided are methods for generating a gene library comprising a plurality of genes partitioned into separate fractions, wherein one or more of the fractions each comprise a subpopulation of nucleic acids that differ from a predetermined sequence by no more than about 1 in 1000 nucleotides. In some embodiments, one or more of the fractions differ from the predetermined sequence by no more than about 1 in 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, 55000, 60000, 70000, 80000, 90000, or 100000 bases.

In various aspects, a method of preparing a gene library comprises synthesizing a plurality of genes having one or more predetermined nucleic acid sequences, amplifying the plurality of genes, and partitioning the plurality of genes into a plurality of fractions. In some embodiments, the genes are synthesized using the methods and substrates described elsewhere herein. In some embodiments, the plurality of genes comprises about or at least about 50, 100, 150, 200, 250, 300, 400, 500, 600, 700, 800, 900, 1000, 6000, 10000 or more genes. In some embodiments, the plurality of genes comprises about or at least about 50, 100, 150, 200, 250, 300, 400, 500, 600, 700, 800, 900 or 1000 genes having different predetermined nucleic acid sequences. In some embodiments, the plurality of fractions comprises about or at least about 10, 20, 30, 40, 50, 100, 150, 200, 250, 300, 400, 500, 600, 700, 800, 900 or more fractions. In some embodiments, each of the plurality of genes has a predetermined nucleic acid sequence comprising about or at least about 100, 200, 300, 400, 500, 600, 700, 800, 900, 1000 or more bases. In some embodiments, the error rate in at least 90% of the fractions is less than about 1 in 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, 55000, 60000, 70000, 80000, 90000, or 100000 bases. In some embodiments, the gene library is generated in less than about 1 month, 1 week, 6 days, 5 days, 4 days, 72 hours, 48 hours, 24 hours, 12 hours or 6 hours. In some embodiments, the plurality of synthesized genes is partitioned into fractions prior to amplification.

In some embodiments, each fraction comprises about or at least about 0.1, 0.2, 0.3, 0.4, 0.5, 1, 1.1, 1.2, 1.3, 1.4, 1.5, 2, 3, 4, 5, 10 or more nucleic acid molecules that are subject to cell-free sorting. Cell-free sorting includes any of the methods described herein, including, for example, methods comprising amplification of nucleic acid molecules within a fraction and sequencing to select clonal populations of nucleic acids. In additional instances, the amplified nucleic acids within each fraction have identical or nearly identical sequences to the parent nucleic acid(s). For example, sequence deviations expected could occur during amplification with a frequency similar to polymerase error rates.

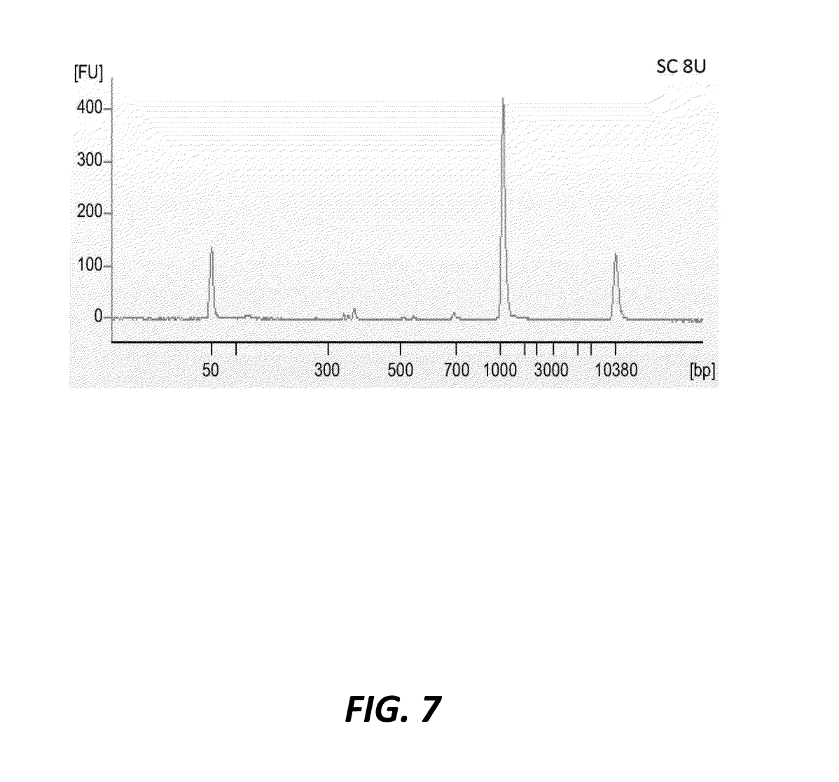

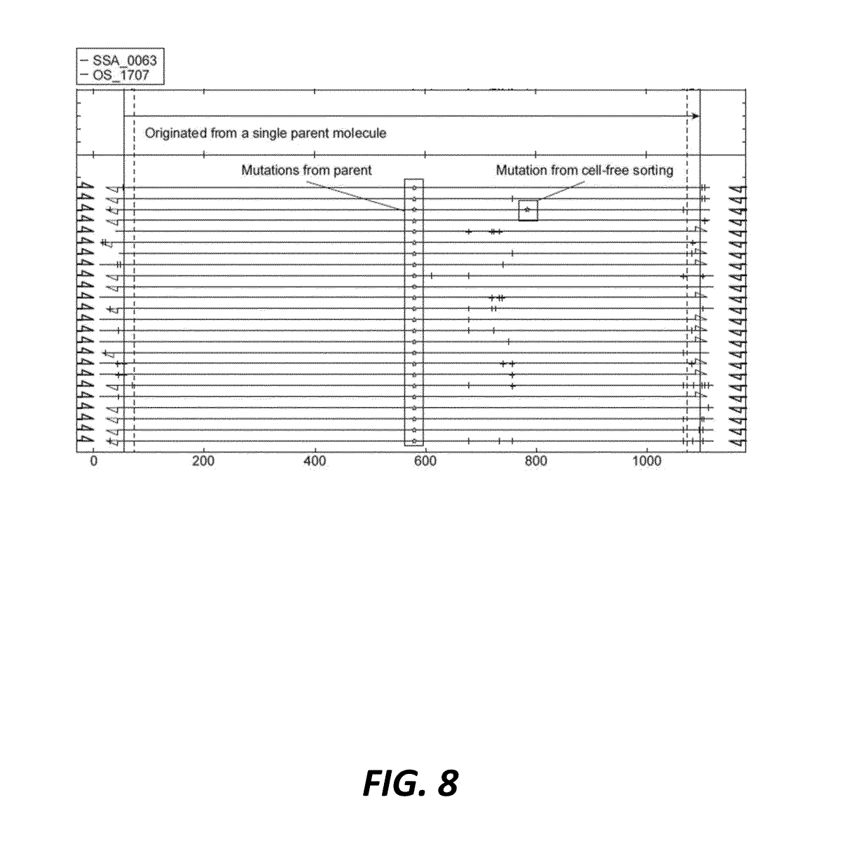

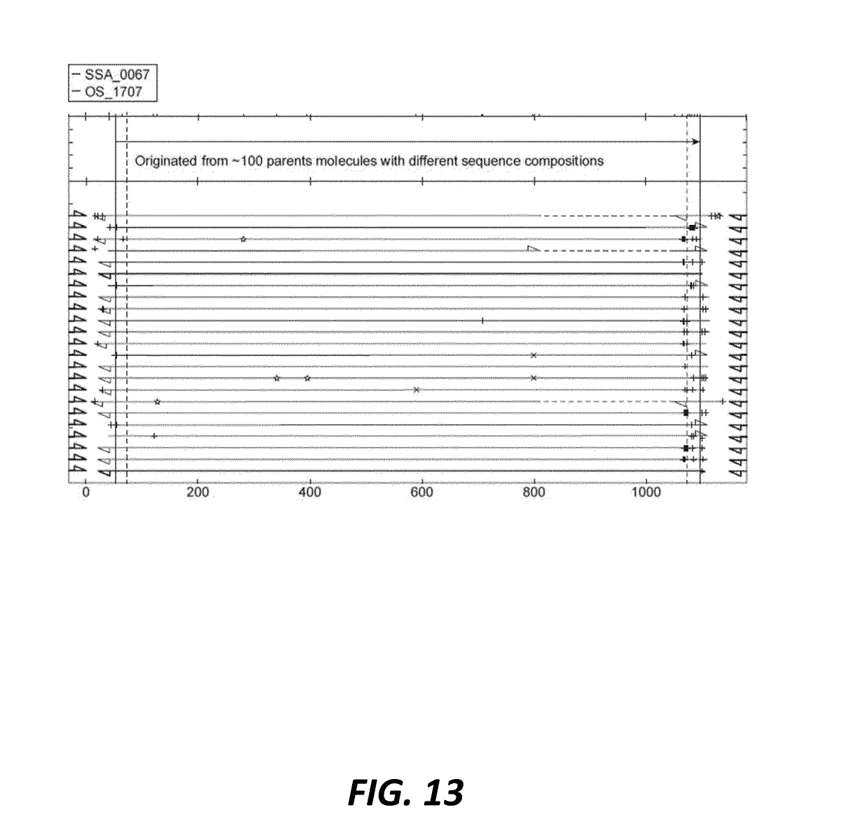

An embodiment of a method of cell free sorting using double-stranded circularized DNA is exemplified by FIGS. 7-13. In this embodiment, a sample of double-stranded target nucleic acids with a heterogeneous sequence population is partitioned using cell-free sorting methods described herein. The sample comprises a subpopulation of sequences having a predetermined desired sequence and a subpopulation of sequences having the predetermined sequence with one or more errors (e.g., mutations). The target sequences are amplified with 5′ uracil containing primers to generate uracil-containing target nucleic acids. An electrophoresis digital trace of the amplified uracil-containing target nucleic acids is shown in FIG. 7. The uracil-containing target nucleic acids are then digested with UDG and EndoVIII to generate 3′ overhangs. The digested target nucleic acids are ligated with an adapter comprising a first strand and a second strand annealed to have 3′ overhangs. The first strand of the adapter has a 5′ phosphate group for ligation to the 3′ end of the first strand of a target nucleic acid, and the first strand of the target nucleic acid has a 5′ phosphate group for ligation to the 3′ end of the first adapter strand, so upon ligation, a continuous, single-strand of circular DNA is generated. The 5′ end of the second target nucleic acid strand has a phosphate group for ligation to the 3′ end of the second adapter strand. The 5′ end of the second adapter strand lacks a 5′ phosphate and has one fewer bases at its 5′ end, so that upon ligation and subsequent hybridization to the continuous circular strand, the second strands form a discontinuous nucleic acid strand with a single nucleotide gap. The hybridized ligation products having a continuous circular strand and a discontinuous strand are referred to as nicked, circularized double-stranded DNA. The nicked, circularized double-stranded DNA products are purified, diluted to femtomolar concentrations, and amplified using RCA. The nicked strand serves as a primer for the template continuous strand. The RCA products are then quantified, diluted, and partitioned into fractions so that, on average, each fraction has a single RCA product. The fractions are each amplified to generate clonal copies of the single parent DNA molecule. Amplification products of 5 clonal fractions are sequenced and the sequence traces shown in FIGS. 8-12. In addition, a sample of RCA products prior to fractioning is sequenced and the sequence trace is shown in FIG. 13. The sequence trace of FIG. 13 shows the heterogeneous nature of the sample prior to cell-free sorting.

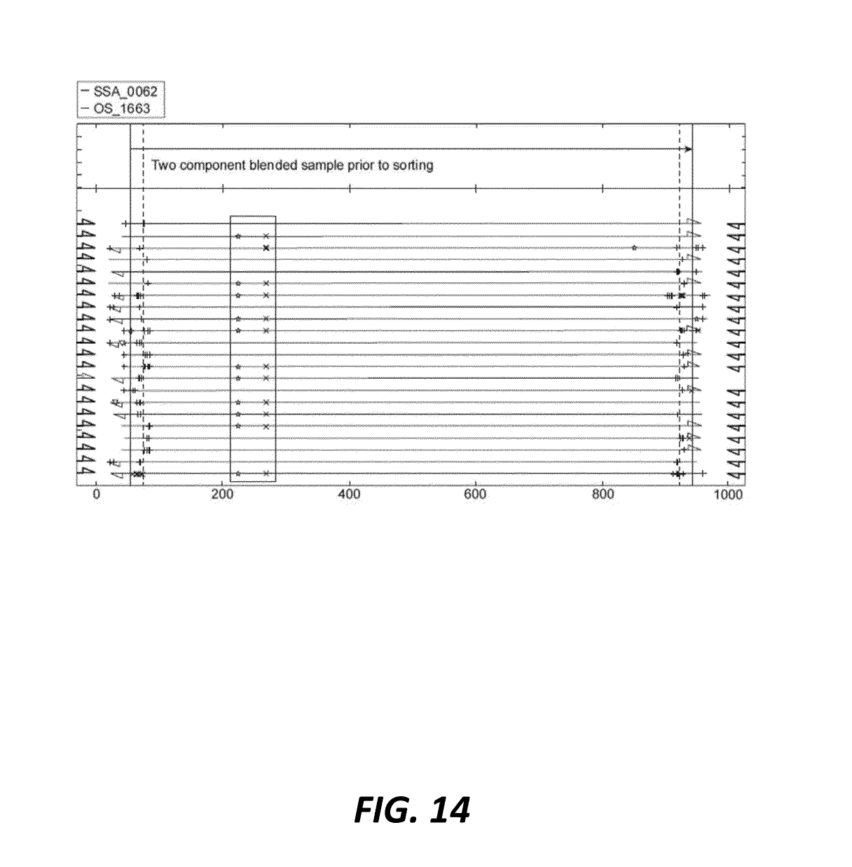

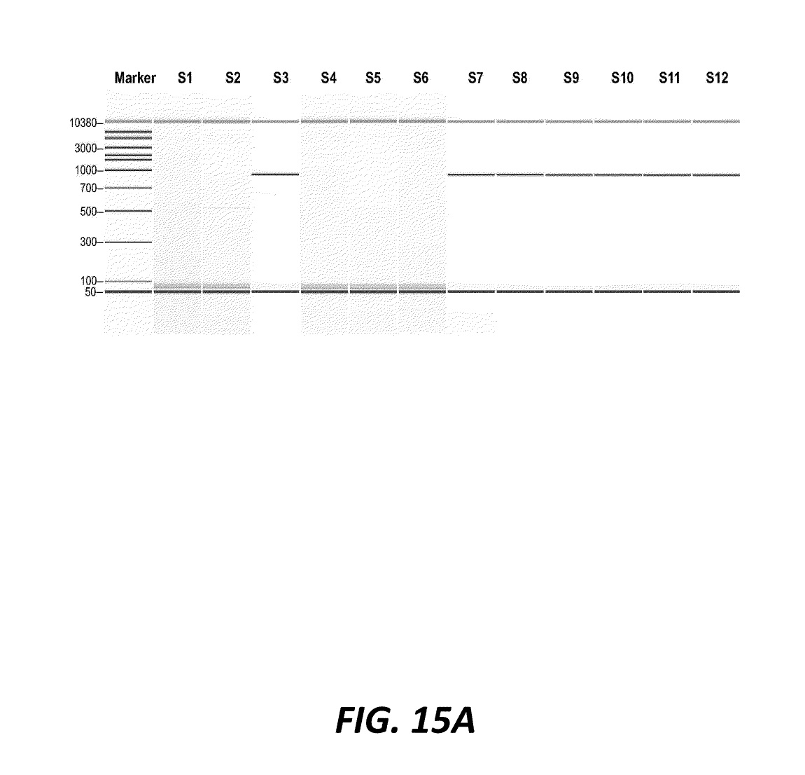

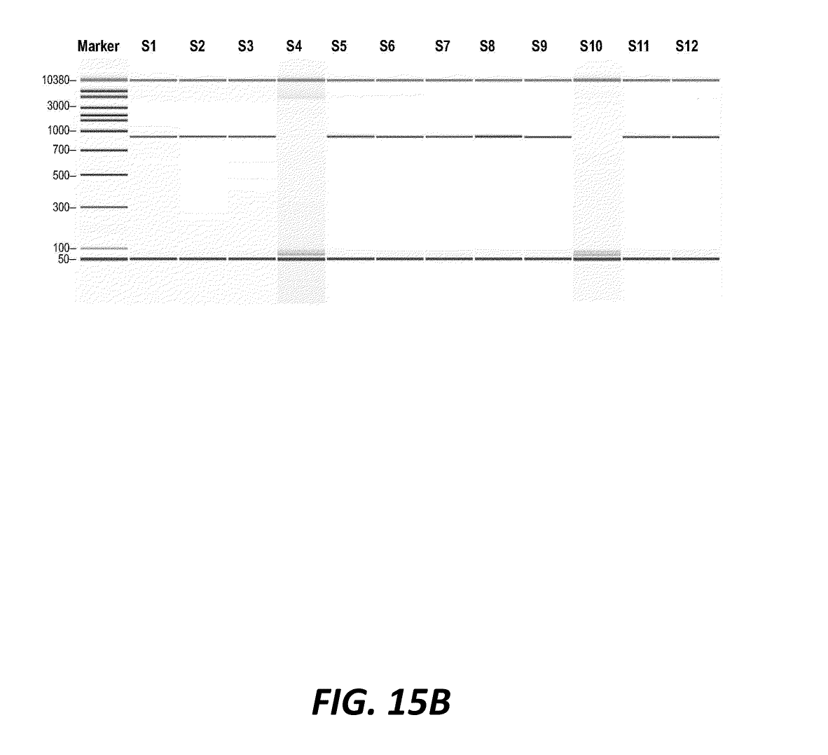

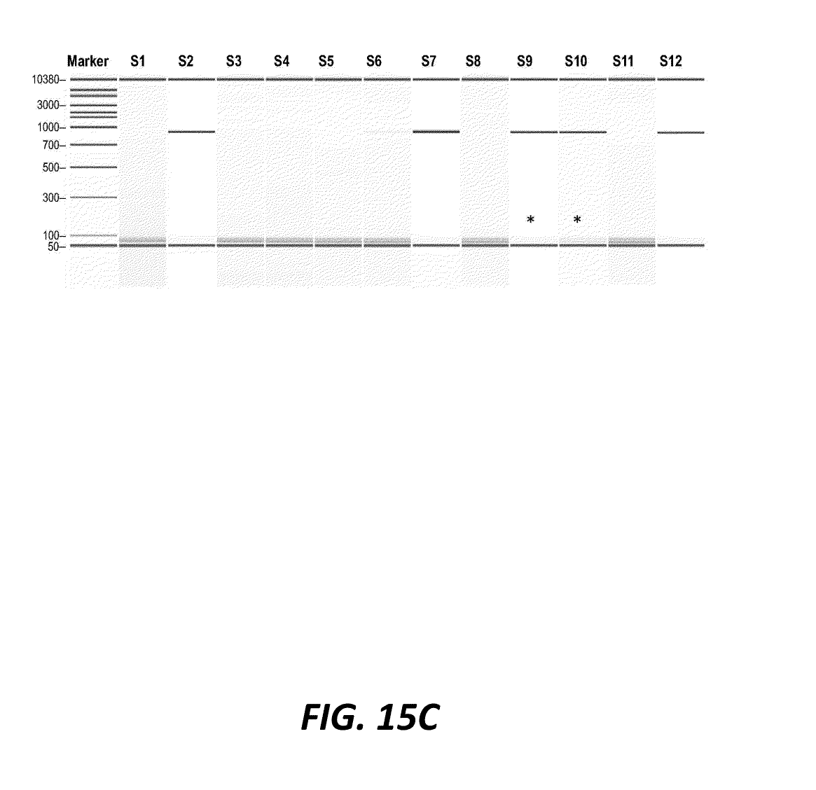

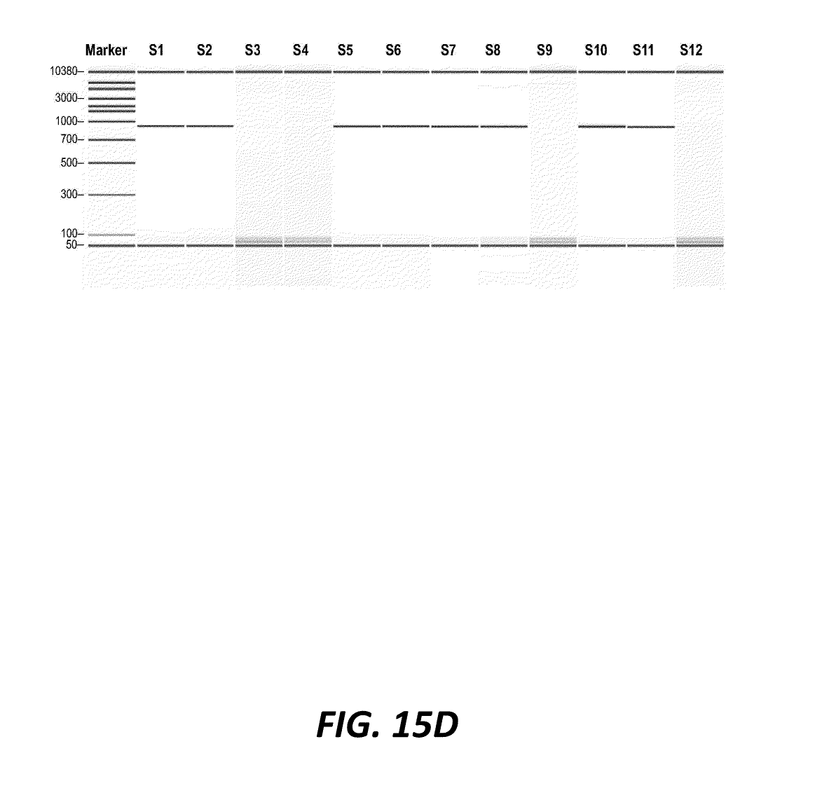

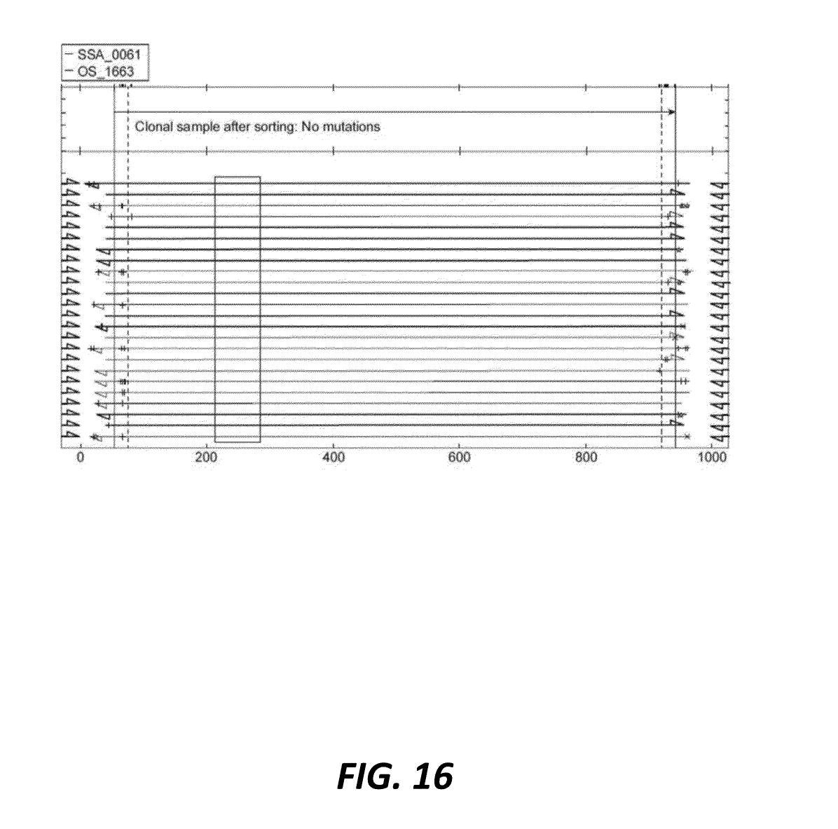

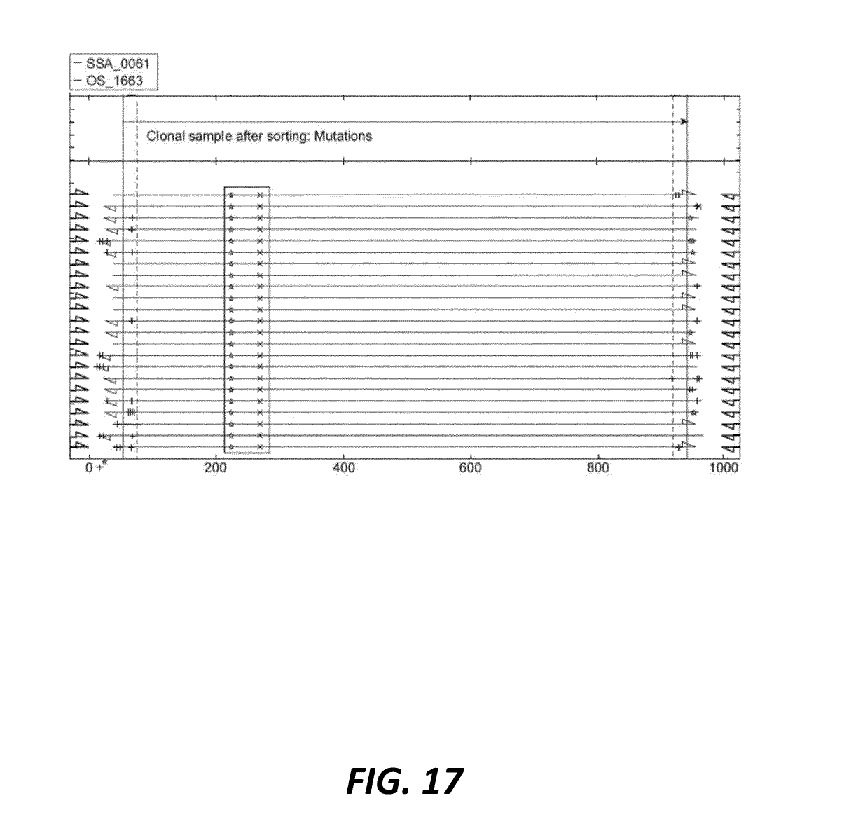

Another embodiment of a method of cell free sorting using double-stranded circularized DNA is exemplified by FIGS. 14-17. In this embodiment, a sample of double-stranded target nucleic acids with a heterogeneous, two-component sequence population is partitioned using the cell-free sorting methods described herein. The sample comprises a subpopulation of sequences having a predetermined desired sequence and a subpopulation of sequences having the predetermined sequence with two mutations. A sequence trace of the sample of target nucleic acids is shown in FIG. 14, where the mutations are indicated by an asterisk and a cross. The sample is diluted and partitioned into 24 fractions so that, on average, each fraction has a single DNA molecule (about 1.2 molecules). Each fraction is subjected to amplification conditions by PCR and the products are visualized by gel electrophoresis, as shown in FIGS. 15A-15B. Similarly, the sample is diluted and partitioned into an additional 24 fractions so that, on average, each fraction has a single DNA molecule (about 0.6 molecules). Each fraction is then subjected to amplification conditions by PCR and the products are visualized by gel electrophoresis, as shown in FIGS. 15C-15D. As shown in FIGS. 15A-15D, some fractions contained product, while others did not, indicating that when performing single molecule partitioning, some fractions will contain a target nucleic acid that can be amplified by PCR, while other fractions will not contain any target nucleic acids. However, as shown in by sequence traces of the amplification products in two separate fractions, FIGS. 16 and 17, at least some of the fractions with amplification products of single molecules have monoclonal populations of nucleic acids (i.e. nucleic acids having the same sequences). The fraction represented in FIG. 16 has a monoclonal population of nucleic acids with the predetermined target sequence. The fraction represented in FIG. 17 has a monoclonal population of nucleic acids with the predetermined target sequence having two mutations.

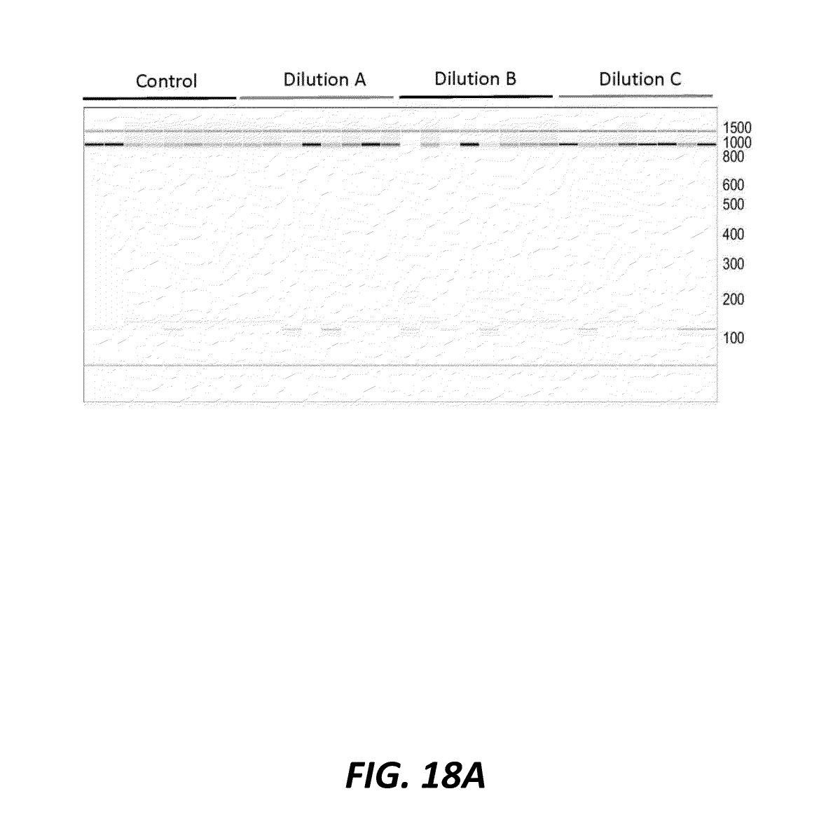

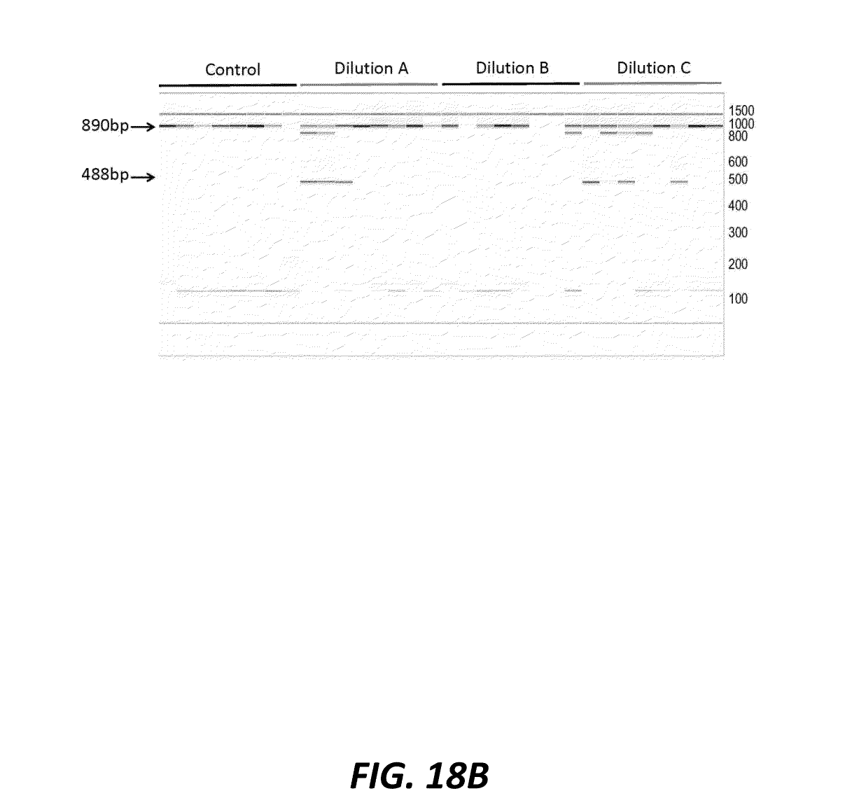

Another embodiment of a method of cell free sorting using double-stranded circularized DNA is exemplified by FIGS. 18A-18B. In this embodiment, a sample of double-stranded target nucleic acids having two different subpopulations of sequences is partitioned into single molecule fractions in nanowells, followed by amplification by RCA. The sample has a first subpopulation of plasmids having a 322 base insert and a second population of plasmids having a 724 base insert. This method is in contrast to the methods embodied in FIGS. 7-13, and FIGS. 14-15, in that the sample is partitioned into single molecule fractions prior to amplification by RCA and that partitioning is performed in small volumes suitable for partitioning into nanowells. For example, the fractions in this embodiment have a volume of 0.3 ul and a RCA reaction is performed within this small volume in a nanowell. After RCA of single molecules in nanowells, samples are extracted and further amplified by PCR for further analysis. FIG. 18B depicts a gel electrophoresis image of a sample of target nucleic acids that are partitioned into about 100 (dilution A), about 10 (dilution B) and about single molecule (dilution C) fractions, followed by RCA and PCR amplification.

For cell free sorting methods that comprise partitioning of target nucleic acid samples prior to RCA amplification, preparing the partitioned fractions for RCA is one factor to be considered for the generation of RCA amplification products. One method for preparing a RCA reaction mixture comprises (a) combining RCA reaction reagents with a primer and a fractionated sample comprising, on average, a single target nucleic acid to generate a first reaction mixture; (b) heating the first reaction mixture to a denaturation temperature; (c) cooling the first reaction mixture of step (b); and (d) combining the first reaction mixture of step (c) with a second reaction mixture comprising DNA polymerase. In one example, a RCA reaction is performed on the RCA reaction mixture prepared using this method, followed by amplification of any RCA amplification products by PCR. FIG. 18B is an image of a gel showing that the presence of PCR amplification products, indicating the presence of RCA amplification products using the RCA reaction mixture prepared by the described method. In some embodiments, the primer comprises 15, 14, 13, 12, 11, 10, 9, 8, 7, 6, 5, or 4 bases. In some cases, the primer is random. Examples of RCA reaction reagents include, without limitation, polymerase buffer, dNTPs, DTT, Tween20, and any combination thereof. Denaturation temperatures include temperatures between about 90° C. to about 105° C. In some cases, a denaturation temperature is about 95° C. In some embodiments, the first reaction mixture is heated to a denaturation temperature for less than about 20, 15, 10, 9, 8, 7, 6, 5, 4, 3, 2 or 1 min. In some cases, the first reaction mixture is heated for 3 minutes. In some embodiments, the first reaction mixture is cooled on ice for more than 1, 2, 3, 4, 5, 6, 7, 8, 9, 10, 15 or 20 minutes. In some embodiments, cooling the first reaction mixture comprises incubating the first reaction mixture on ice. In some cases, the first reaction mixture is cooled on ice for 5 minutes. In some embodiments, the DNA polymerase is phi29 DNA polymerase. In some embodiments, the second reaction mixture further comprises BSA and/or pyrophosphatase.

A second method for preparing a RCA reaction mixture comprises (a) providing a fractionated sample comprising, on average, a single target nucleic acid; (b) heating the fractionated sample to a denaturation temperature; (c) cooling the fractionated sample of step (b); (d) combining RCA reaction reagents with a DNA polymerase to generate a first reaction mixture and incubating the first reaction mixture at room temperature; and (e) combining the fractionated sample of step (c) with the reaction mixture of step (d) and a primer. In this case, in contrast to the prior example, (1) the RCA step occurs after fractionation and (2) RCA reagents are pre-incubated at room temperature. In one example, a RCA reaction is performed on the RCA reaction mixture, followed by amplification of any RCA amplification products by PCR. FIG. 18A is an image of a gel that does not show the presence of any PCR amplification products, indicating that the likely absence of RCA products. In some embodiments, the primer comprises 15, 14, 13, 12, 11, 10, 9, 8, 7, 6, 5, or 4 bases. In some cases, the primer is random. Examples of RCA reaction reagents include, without limitation, polymerase buffer, dNTPs, DTT, Tween20, BSA, pyrophosphatase, and any combination thereof Denaturation temperatures include temperatures between about 90° C. to about 105° C. In some cases, a denaturation temperature is about 95° C. In some embodiments, the fractionated sample is heated to a denaturation temperature for less than about 20, 15, 10, 9, 8, 7, 6, 5, 4, 3, 2 or 1 min. In some cases, the fractionated sample is heated for 3 minutes. In some embodiments, the fractionated sample is cooled on ice for more than 1, 2, 3, 4, 5, 6, 7, 8, 9, 10, 15 or 20 minutes. In some embodiments, cooling the fractionated sample comprises incubating the first reaction mixture on ice. In some cases, the fractionated sample is cooled on ice for 5 minutes. In some embodiments, the DNA polymerase is phi29 DNA polymerase. In some embodiments, the first reaction mixture is incubated at room temperature for about 5 to 30 minutes, e.g., 10 minutes.

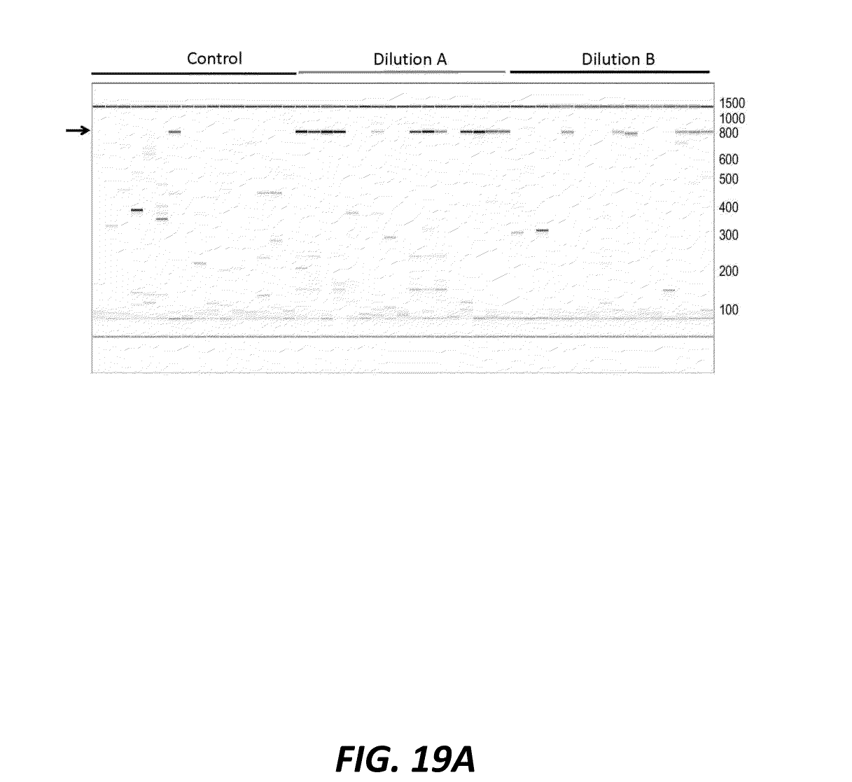

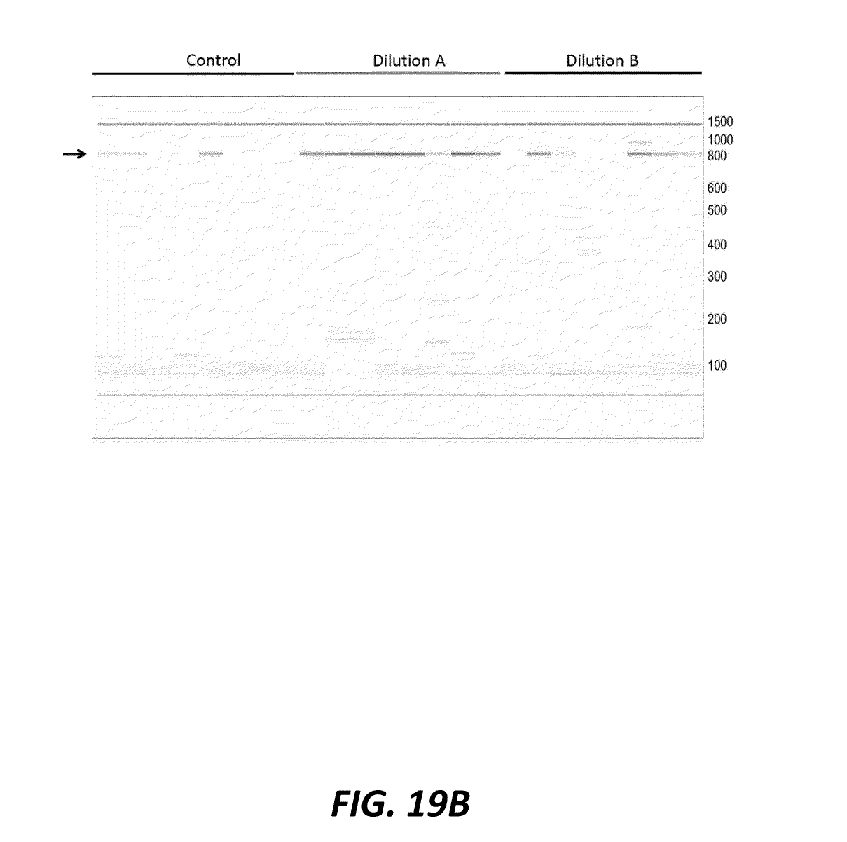

A further embodiment of a method of cell free sorting using double-stranded circularized DNA is exemplified by FIG. 19A-19B. In this embodiment, a heterogeneous two-component sample of double-stranded plasmid target nucleic acids are partitioned into single molecule fractions in nanowells, followed by amplification by RCA. The sample has a subpopulation of plasmids with inserts having a predetermined sequence and a subpopulation of plasmids with inserts having the predetermined sequence and one mutation. The sample is fractionated into nanowells so that each well has, on average, about 5 or about 1 DNA molecules. RCA is performed on each fraction, followed by PCR. FIG. 19A-19B show the PCR amplification products, indicating that some fractions contained DNA products and by extension, parent DNA molecules from partitioning. In particular, the electrophoresis gel of FIG. 19A shows PCR products that were amplified from RCA products that are amplified in nanowells having, on average about 5 (dilution A) or about 1 (dilution B) parent DNA molecules. The electrophoresis gel of FIG. 19B shows PCR products that are amplified from RCA products that are amplified in tubes having, on average about 5 (dilution A) or about 1 (dilution B) parent DNA molecules. Sequencing of the PCR amplification products from selected fractions indicate that each of the sequenced fractions have a monoclonal population of nucleic acids (all copies have either the predetermined sequence or the predetermined sequence with the base mutation).

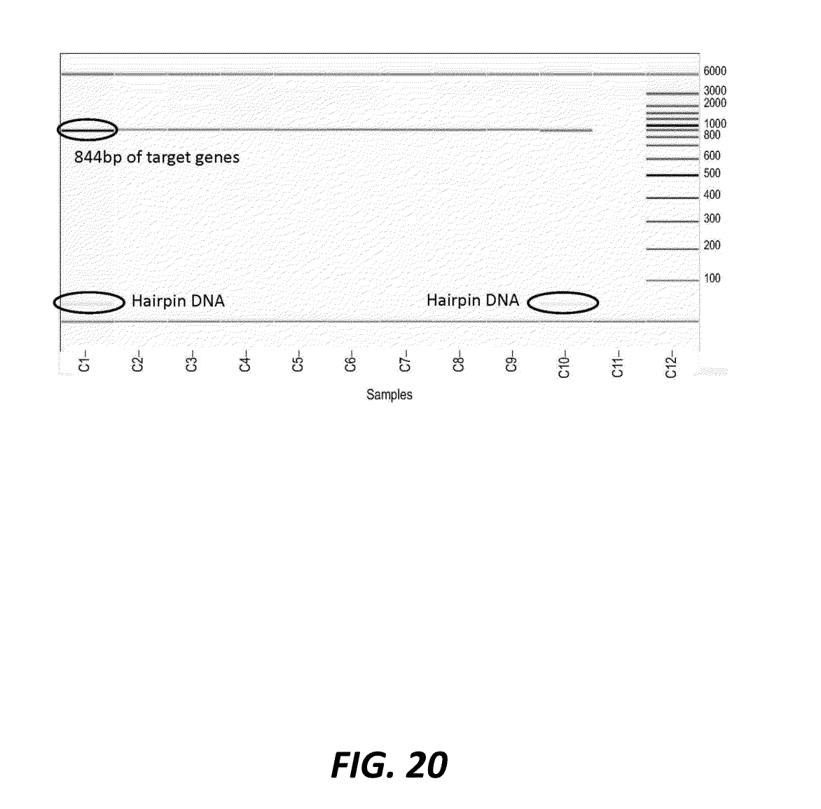

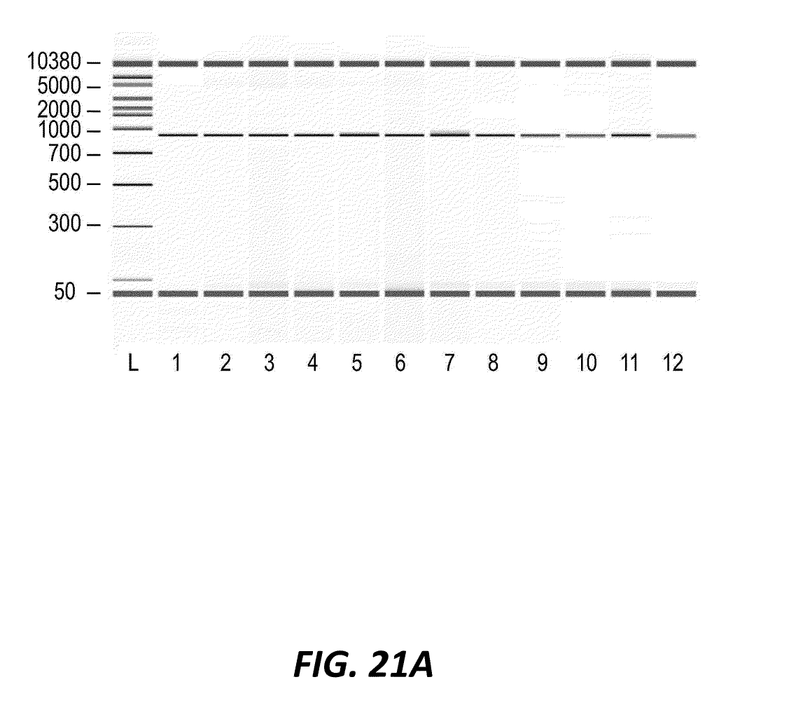

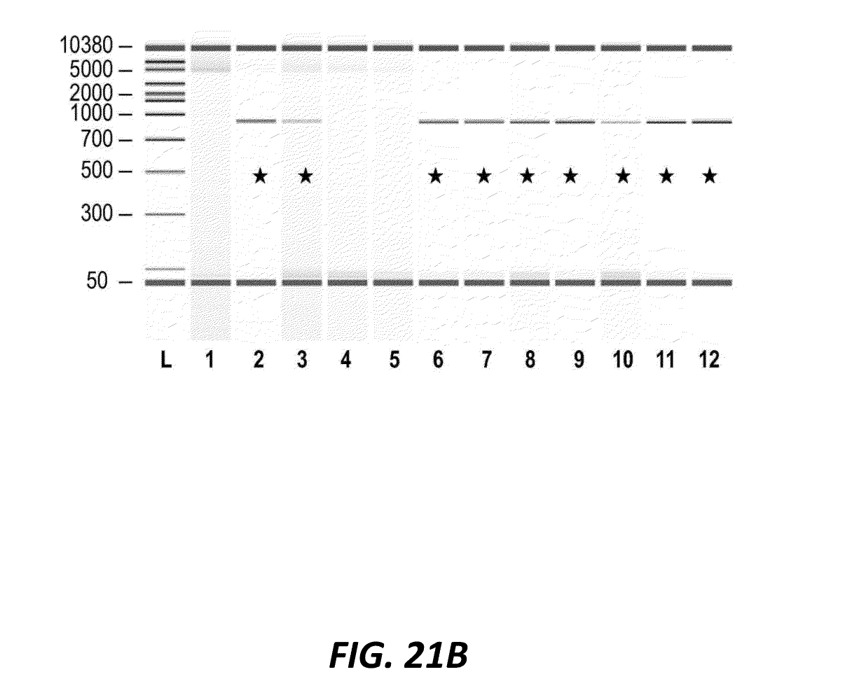

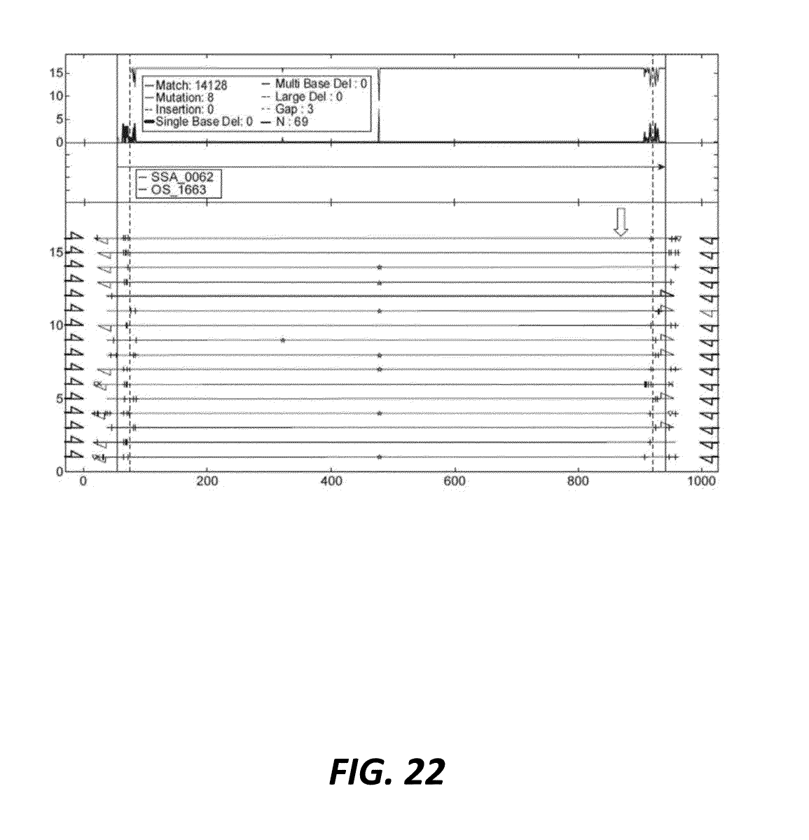

An embodiment of a method of cell-free sorting using target nucleic acids circularized using DNA hairpins is exemplified by FIGS. 20-30. In this embodiment, a sample of double-stranded target nucleic acids has a subpopulation of nucleic acids having a predetermined sequence and a subpopulation of nucleic acids with the predetermined sequence and one mutation. The sample of target nucleic acids are amplified with uracil containing primers to generate target nucleic acids with 5′ uracil bases. The target nucleic acids are treated with UDG and EndoVIII to generate 3′ overhangs. To generate single-stranded circular DNA, e.g., bell DNA, the target nucleic acids are hybridized and ligated to hairpin DNA. FIG. 20 shows a gel electrophoresis of target nucleic acids ligated to DNA hairpins. Single-stranded target nucleic acids are amplified by RCA, diluted and partitioned into fractions having, on average about 10 or 1 DNA molecules per fraction. Each fraction is then amplified by PCR. FIG. 21A shows a gel electrophoresis of fractions having about 10 molecules of parent DNA that are amplified by RCA followed by PCR. FIG. 21B shows a gel electrophoresis of fractions having about 1 molecule of parent DNA that are amplified by RCA followed by PCR. Sequencing traces of the PCR products shown in FIG. 21B are provided in FIGS. 22-30. FIGS. 22-30 show that a population of heterogeneous bell DNA molecules can be amplified, separated into single molecule fractions, and amplified to generate fractions having monoclonal populations of nucleic acids. One benefit of using bell like DNA for cell-free sorting is that RCA amplification products are compact and allow for handling in small volumes that facilitate partitioning into single molecule fractions.

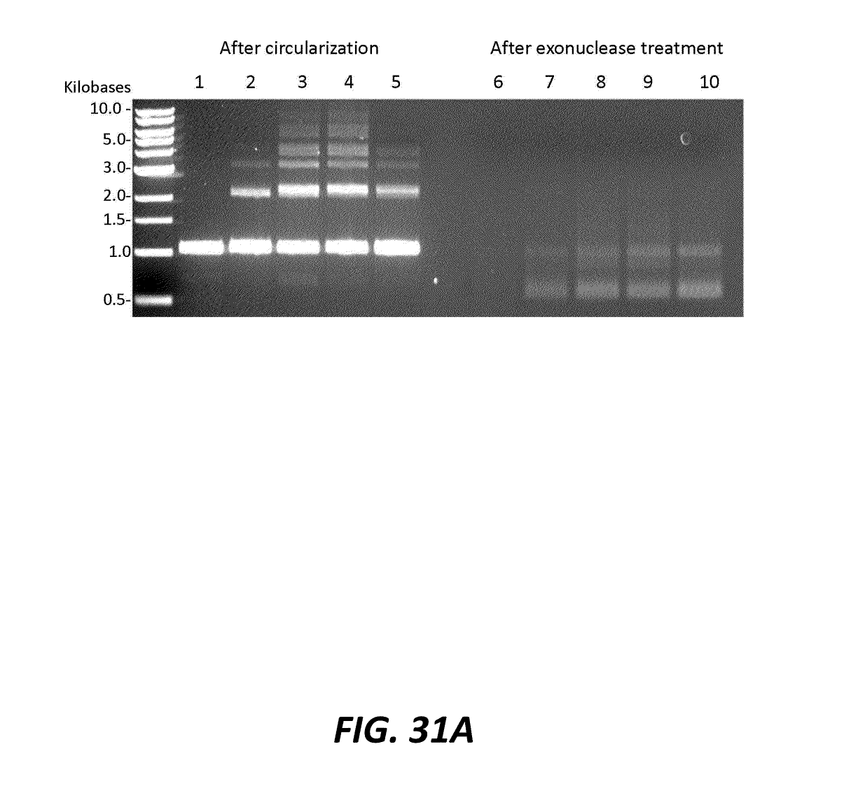

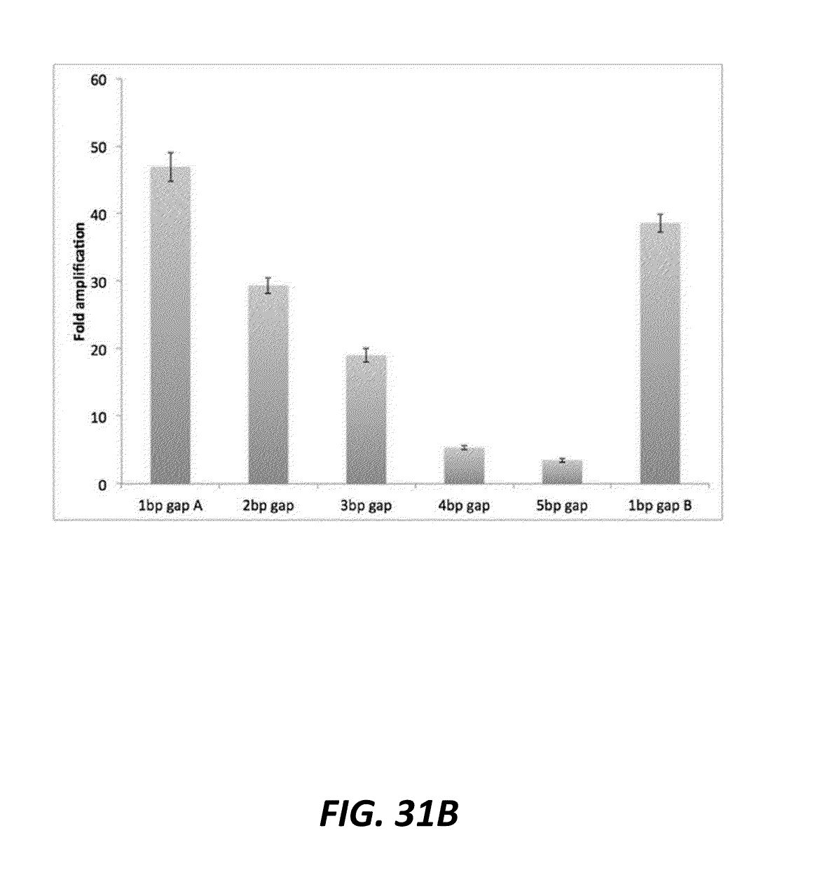

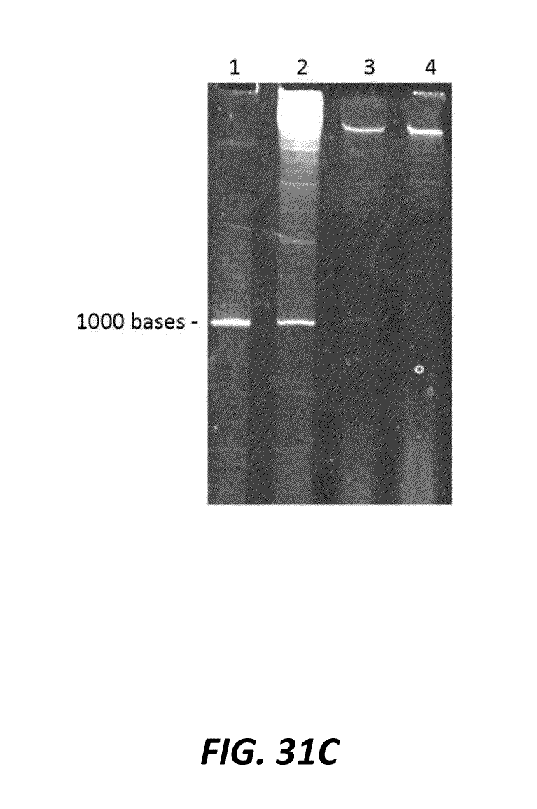

In some embodiments, target nucleic acids are circularized by self-ligation for cell-free sorting. FIG. 31A-31C illustrates embodiments for generating a circularized target nucleic acid. One method for generating a circularized target nucleic acid comprises generating sticky ends on both ends of the target. For the embodiment illustrated in FIG. 31A, double-stranded target nucleic acids (1 kbp) are self-ligated with sticky ends and treated with exonuclease to remove non-circularized DNA. The sticky ends are generated by amplification of the target nucleic acids with uracil containing primers, followed by enzymatic digestion of PCR products with UDG and EndoVIII. FIG. 31A shows the circularization of the target nucleic acids using sticky ends having overhangs of 4 (lane 2), 6 (lane 3), 8 (lane 4), and 10 (lane 5) bases; and target nucleic acids lacking sticky ends (control). The circularized target nucleic acids are visualized after exonuclease treatment and are shown in lanes 6-10 (corresponding to lanes 1-5). Target nucleic acids circularized by sticky end self-ligation serve as templates for RCA. FIG. 31B depicts a plot of the amplification of self-ligated target nucleic acids having various gap sizes. Another method for generating a circularized target nucleic acid comprises blunt end self-ligation. FIG. 31C demonstrates an example of a target nucleic acid (1 kbp) circularized using blunt end self-ligation. In this example, the target nucleic acid is amplified with a first primer having a 5′ phosphate and a second primer lacking a 5′ phosphate and a few 5′ bases so that upon ligation, one strand would have fewer bases, generating a nick in a double-stranded, circularized DNA. The second primer also comprised 5′ phosphorothioated bonds to resist digestion by exonuclease treatment.

Generation of Source Material for Cell-Free Sorting and Cloning

The cell-free sorting and cloning methods described herein is suitable for both enzymatically or non-enzymatic generated nucleic acids starting material. Exemplary sources of nucleic acid starting material include, without limitation, cellular extracts, PCR amplification products, and chemical oligonucleic acid synthesis reactions. In one example, de novo synthesized oligonucleic acids referenced herein are synthesized on a device comprising a substrate having distinct regions functionalized to support nucleic acid attachment and elongation. In such a case, distinct regions include clusters, where each cluster comprises a plurality of loci, with each locus optionally configured to support the synthesis of an oligonucleic acid encoding for a particular predetermined sequence.

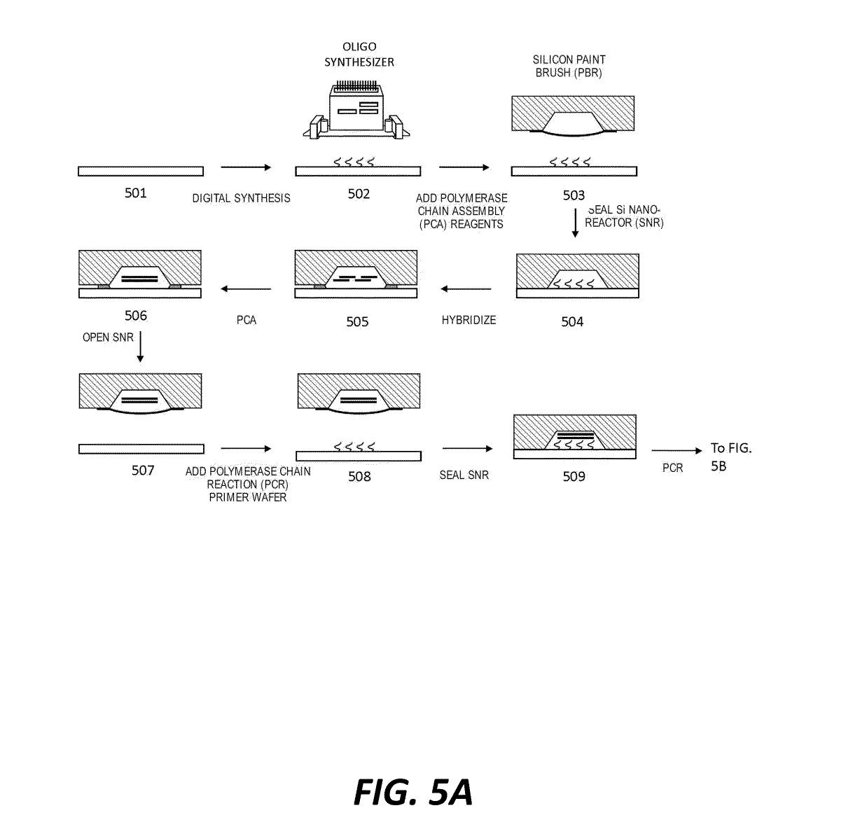

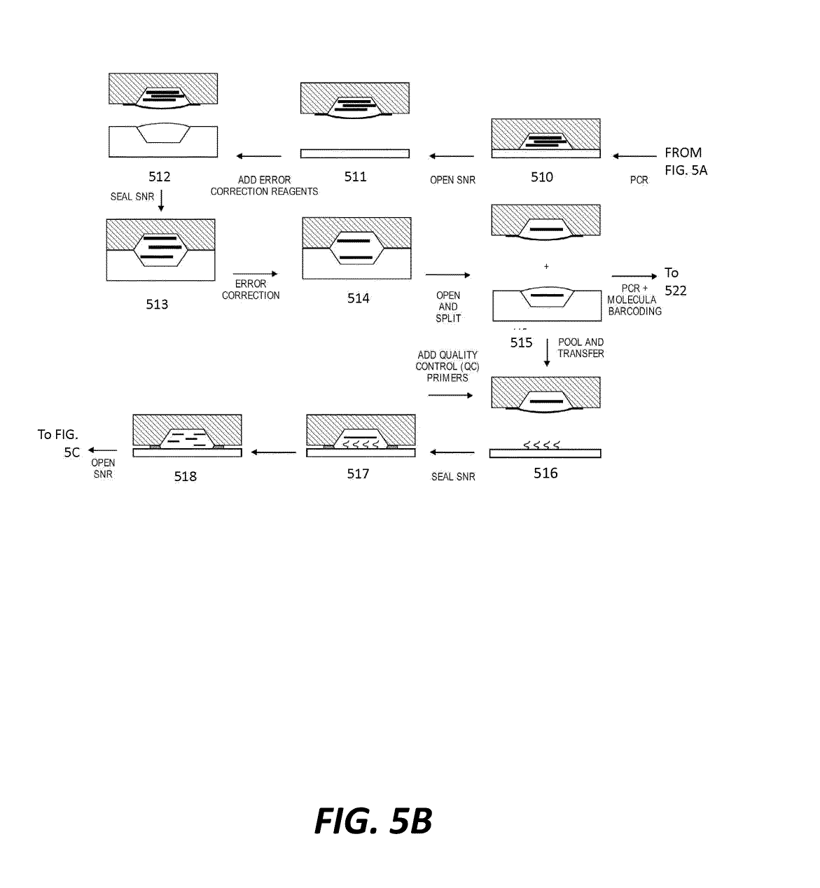

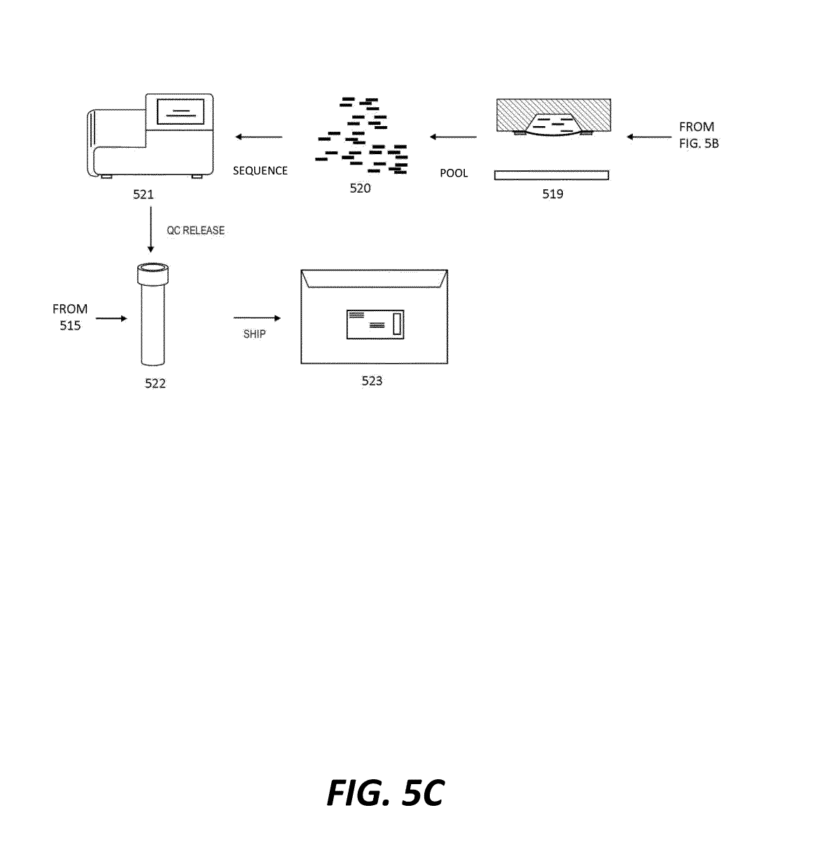

FIGS. 5A-5C illustrates an exemplary process workflow for the de novo synthesis of a population of large oligonucleic acids. Prior to de novo synthesis, an intended nucleic acid sequence or group of nucleic acid sequences is predetermined. After de novo synthesis, the synthesized oligonucleic acids are sorted into subpopulations having the desired, predetermined synthesized sequence. The workflow of FIGS. 5A-5C is divided generally into phases: (1) de novo synthesis of a single-stranded oligonucleic acid library, (2) joining oligonucleic acids to form larger fragments, (3) error correction, (4) quality control, and (5) shipment. Nucleic acid sorting is suitably performed between one or more of these phases, or as a part of a phase, for example, during error correction or quality control.

Various suitable methods are known for generating high density oligonucleic acid arrays. In the workflow example, a substrate surface is provided. In the example, chemistry of the surface is altered in order to improve the oligonucleic acid synthesis process. Areas of low surface energy are generated to repel liquid while areas of high surface energy are generated to attract liquids. The surface itself may be in the form of a planar surface or contain variations in shape, such as protrusions or nanowells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of oligonucleic acid arrays is generated on a solid support and utilizes single nucleotide extension processes to extend multiple oligomers in parallel. A device, such as an oligonucleic acid synthesizer, is designed to release reagents in a step wise fashion such that multiple oligonucleic acids extend, in parallel, one residue at a time to generate oligomers with a predetermined nucleic acid sequence 502. In some cases, oligonucleic acids are cleaved from the surface at this stage. Cleavage may include gas cleavage, e.g., with ammonia or methylamine.

The generated oligonucleic acid libraries are placed in a reaction chamber. In this exemplary workflow, the reaction chamber (also referred to as “nanoreactor”) is a silicon coated well containing PCR reagents lowered onto the oligonucleic acid library 503. Prior to or after the sealing 504 of the oligonucleic acids, a reagent is added to release the oligonucleic acids from the substrate. In the exemplary workflow, the oligonucleic acids are released subsequent to sealing of the nanoreactor 505. Once released, fragments of single-stranded oligonucleic acids hybridize in order to span an entire long range sequence of DNA. Partial hybridization 505 is possible because each synthesized oligonucleic acid is designed to have a small portion overlapping with at least one other oligonucleic acid in the pool.

After hybridization, a PCA reaction is commenced. During the polymerase cycles, the oligonucleic acids anneal to complementary fragments and gaps are filled in by a polymerase. Each cycle increases the length of various fragments randomly depending on which oligonucleic acids find each other. Complementarity amongst the fragments allows for forming a complete large span of double-stranded DNA 506.

After PCA is complete, the nanoreactor is separated from the substrate 507 and positioned for interaction with a substrate having primers for PCR 508. After sealing, the nanoreactor is subject to PCR 509 and the larger nucleic acids are amplified. After PCR 510, the nanochamber is opened 511, error correction reagents are added 512, the chamber is sealed 513 and an error correction reaction occurs to remove mismatched base pairs and/or strands with poor complementarity from the double-stranded PCR amplification products 114. The nanoreactor is opened and separated 515. Error corrected product is next subject to additional processing steps, such as PCR, nucleic acid sorting, and/or molecular bar coding, and then packaged 522 for shipment 523.

In some cases, quality control measures are taken. After error correction, quality control steps include, for example, interaction with a wafer having sequencing primers for amplification of the error corrected product 516, sealing the wafer to a chamber containing error corrected amplification product 517, and performing an additional round of amplification 518. The nanoreactor is opened 519 and the products are pooled 520 and sequenced 521. In some cases, nucleic acid sorting is performed prior to sequencing. Cell-free sorting and cloning methods disclosed herein are applicable to this phase in the workflow. After an acceptable quality control determination is made, the packaged product 522 is approved for shipment 523.

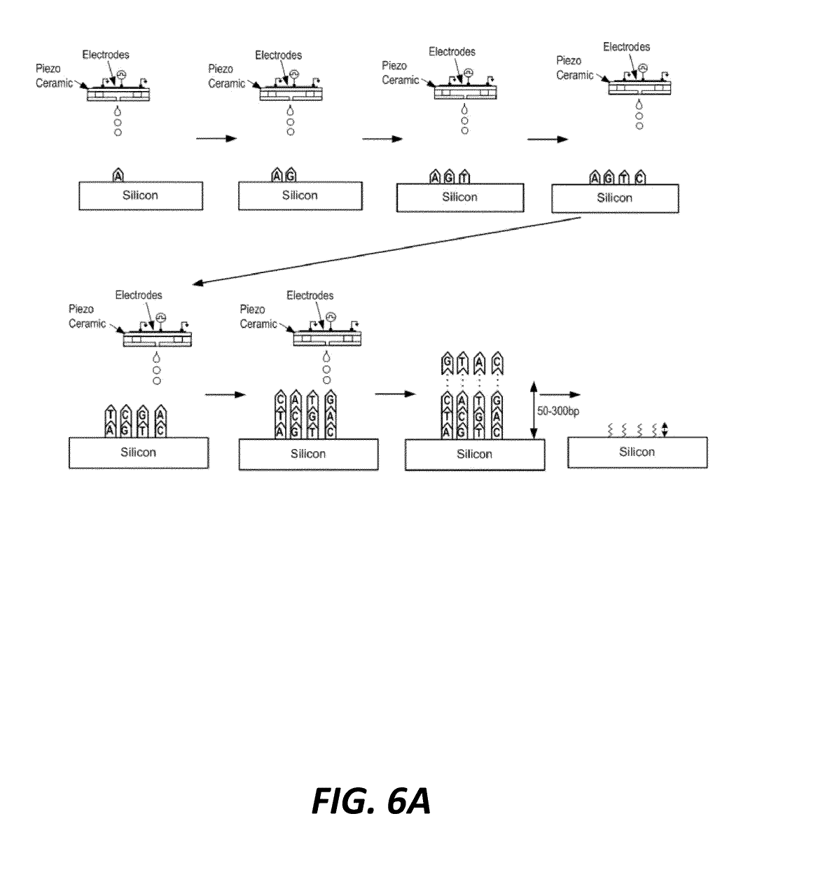

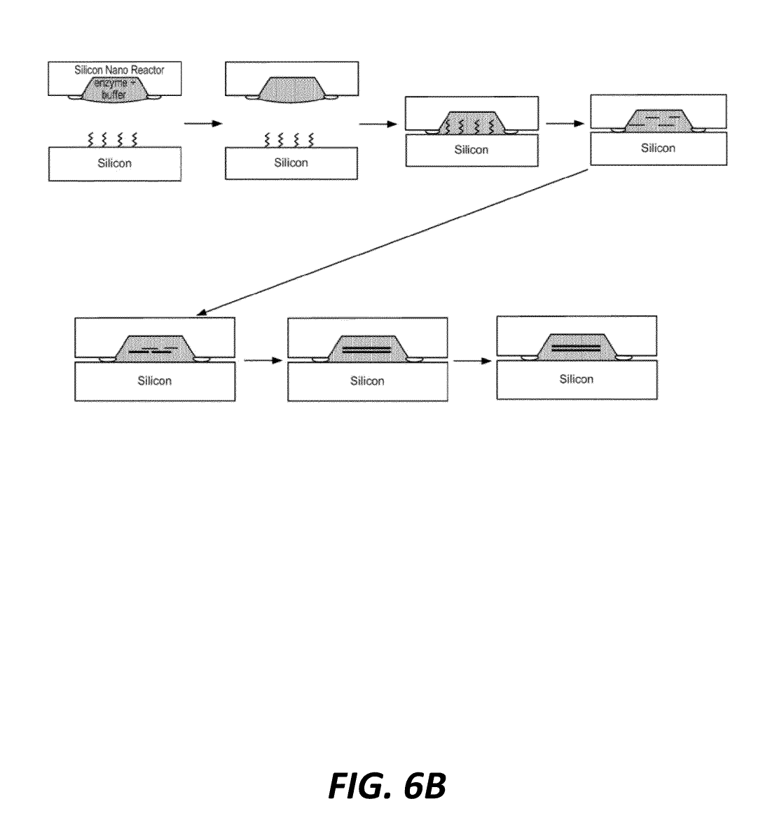

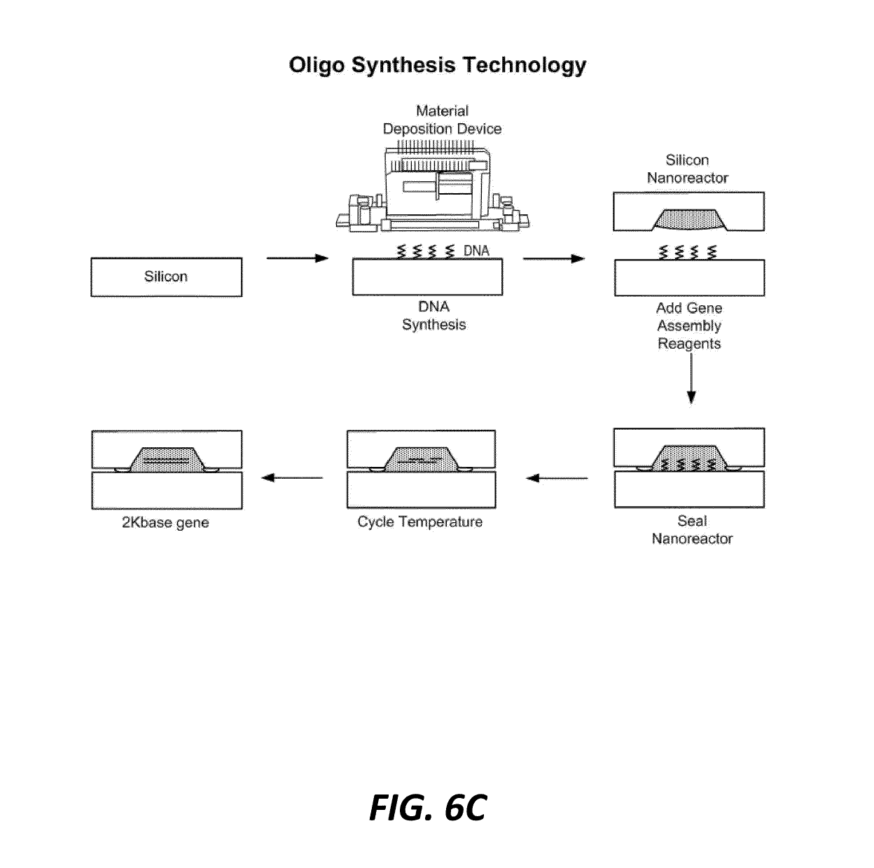

FIGS. 6A-6C illustrates an exemplary process workflow for synthesis of large oligonucleic acids, such as genes, which are targets for nucleic acid sorting using cell-free methods. FIG. 6A illustrates an example process for de novo synthesis of a single-stranded oligonucleic acid library on a substrate using an oligonucleic acid synthesizer. In FIG. 6A, droplets are released from a device having a piezo ceramic material and electrodes to convert electrical signals into a mechanical signal for releasing droplets. Droplets are release to specific locations on the surface of a wafer and droplets comprise reagents for the extension reaction. FIG. 6B illustrates an example process for joining the synthesized oligonucleic acids to form larger fragments in a resolved enclosure or nanoreactor. In this example, a silicon nanoreactor containing enzymes and buffers is deposited on the surface having synthesized oligonucleic acids. Oligonucleic acids are released from the surface by a liquid or gas step. When the nanoreactor makes contact with the oligonucleic acids, they disperse in the fluid. After annealing and PCA reactions, a longer nucleic acid is formed.

FIG. 6C illustrates an exemplary process for gene synthesis using a device, such as an oligonucleic acid synthesizer, to de novo synthesize a library of oligonucleic acids for assembly in a sealed nanoreactor. In situ preparation of oligonucleic acid arrays is generated on the substrate, such as a silicon functionalized substrate, utilizing a single nucleotide extension process to extend multiple oligomers. The device releases reagents in a step wise fashion such that multiple oligonucleic acids extend, in parallel, one residue at a time to generate oligomers with a predetermined nucleic acid sequence. The generated oligonucleic acid libraries are placed in a reaction chamber. In this exemplary workflow, the reaction chamber (also referred to as “nanoreactor”) is a silicon coated well containing PCR reagents and lowered onto the oligonucleic acid library generated during de novo synthesis. Prior to or after the sealing of the nanoreactor with the substrate having the oligonucleic acid library, a reagent is added to release the oligonucleic acids from the substrate. Once released, fragments of the synthesized single-stranded oligonucleic acids hybridize in order to span an entire long range sequence of DNA. Partial hybridization is possible because each synthesized oligonucleic acid is designed to have a small portion overlapping with at least one other oligonucleic acid in the pool. After hybridization, a PCA reaction is commenced. During the polymerase cycles, the oligonucleic acids anneal to complementary fragments and gaps are filled in by a polymerase. Each cycle increases the length of various fragments randomly depending on which oligonucleic acids find each other. Complementarity amongst the fragments allows for forming a complete large span of double-stranded DNA, for example, a double-stranded DNA having 2000 base pairs as shown in FIG. 2C. The double-stranded DNA products are clonally sorted to separate fractions having the predetermined desired synthesis sequence and fractions having one or more errors.

Oligonucleic acids are synthesized on a substrate described herein using a system comprising an oligonucleic acid synthesizer that deposits reagents necessary for synthesis. Reagents for oligonucleic acid synthesis include, for example, reagents for oligonucleic acid extension and wash buffers. As non-limiting examples, the oligonucleic acid synthesizer deposits coupling reagents, capping reagents, oxidizers, de-blocking agents, acetonitrile and gases such as nitrogen gas. In addition, the oligonucleic acid synthesizer optionally deposits reagents for the preparation and/or maintenance of substrate integrity.

In some embodiments, a substrate having a plurality of clusters is configured to seal with a capping element having a plurality of caps, wherein when the substrate and capping element are sealed, each cluster is separate from another cluster to form separate resolved reactors for each cluster. In some instances, the capping element is not present in the system or is present and stationary. A resolved reactor is configured to allow for the transfer of fluid, including oligonucleic acids and/or reagents, from the substrate to the capping element and/or vice versa. Fluid may pass through either or both the substrate and the capping element and includes, without limitation, coupling reagents, capping reagents, oxidizers, de-blocking agents, acetonitrile and nitrogen gas. The oligonucleic acid synthesizer of an oligonucleic acid synthesis system may comprise a plurality of material deposition devices, for example from about 1 to about 50 material deposition devices. Each material deposition device, in various instances, deposits a reagent component that is different from another material deposition device. In some cases, each material deposition device has a plurality of nozzles, where each nozzle is optionally configured to correspond to a cluster on a substrate. For example, for a substrate having 256 clusters, a material deposition device has 256 nozzles and 100 μm fly height. In some cases, each nozzle deposits a reagent component that is different from another nozzle.

Synthesis of Target Nucleic Acids

In some embodiments, the error rates for synthesized oligonucleic acids is less than about 1 in 1000, less than about 1 in 2000, less than about 1 in 3000 or less than about 1 in 5000. In some embodiments, these error rates are for at least 50%, 60%, 70%, 80%, 90%, 95%, 98%, 99%, 99.5%, or more of the oligonucleic acids synthesis products. In some embodiments, these error rates are for 100% of the oligonucleic acids synthesis products. The term error rate as used in this context, refers to a comparison of the collective sequence of synthesized nucleic acids compared to the aggregate sequence of a predetermined longer nucleic acid, e.g., a gene.

In some instances, a surface of the substrate of a device is coated with a layer of material comprising an active functionalization agent. An active functionalization agent is one that binds to the surface of the substrate and also binds to a nucleic acid monomer, thereby supporting a coupling reaction to the surface. In some cases, active functionalization agents are molecules having a hydroxyl group available for interacting with a nucleoside in a coupling reaction. In some instances, a surface of the substrate is coated with a layer of material comprising a passive functionalization agent. A passive functionalization agent or material binds to the surface of the substrate but does not efficiently bind to nucleic acid, thereby preventing nucleic acid attachment at sites where passive functionalization agent is bound. In some cases, active functionalization agents are molecules lacking an available hydroxyl group for interacting with a nucleoside in a coupling reaction.

Oligonucleic acids synthesized using the methods and/or substrates described herein comprise, in various embodiments, at least about 50, 60, 70, 75, 80, 90, 100, 120, 150, 200, 300, 400, 500, 600, 700, 800 or more bases. In some embodiments, a library of oligonucleic acids are synthesized, wherein a population of distinct oligonucleic acids are assembled to generate a larger nucleic acid comprising at least about 500 to; 1,000; 2,000; 3,000; 4,000; 5,000; 6,000; 7,000; 8,000; 9,000; 10,000; 11,000; 12,000; 13,000; 14,000; 15,000; 16,000; 17,000; 18,000; 19,000; 20,000; 25,000; 30,000; 40,000; or 50,000 bases. In some embodiments, methods for oligonucleic acid synthesis described herein generate an oligonucleic acid library comprising at least 500; 1,000; 5,000; 10,000; 20,000; 50,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 2,200,000; 2,400,000; 2,600,000; 2,800,000; 3,000,000; 3,500,000; 4,00,000; or 5,000,000 distinct oligonucleic acids.

In some embodiments, libraries of oligonucleic acids are synthesized in parallel on substrate. For example, a substrate comprising about or at least about 100; 1,000; 10,000; 100,000; 1,000,000; 2,000,000; 3,000,000; 4,000,000; or 5,000,000 resolved loci is able to support the synthesis of at least the same number of distinct oligonucleic acids, wherein oligonucleic acid encoding a distinct sequence is synthesized on a resolved locus. In some embodiments, a library of oligonucleic acids are synthesized on a substrate with low error rates described herein in less than about three months, two months, one month, three weeks, 15, 14, 13, 12, 11, 10, 9, 8, 7, 6, 5, 4, 3, 2 days, 24 hours or less. In some embodiments, larger nucleic acids assembled from an oligonucleic acid library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some embodiments, oligonucleic acid error rate is dependent on the efficiency of one or more chemical steps of oligonucleic acid synthesis. In some cases, oligonucleic acid synthesis comprises a phosphoramidite method, wherein a base of a growing oligonucleic acid chain is coupled to phosphoramidite. In some embodiments, coupling efficiency of the base is related to error rate. For example, higher coupling efficiency correlates to lower error rates. In some cases, the substrates and/or synthesis methods described herein allow for a coupling efficiency greater than 98%, 98.5%, 99%, 99.5%, 99.6%, 99.7%, 99.8%, 99.9%, 99.95%, 99.96%, 99.97%, 99.98%, or 99.99%. In some cases, an oligonucleic acid synthesis method comprises a double coupling process, wherein a base of a growing oligonucleic acid chain is coupled with a phosphoramidite, the oligonucleic acid is washed and dried, and then treated a second time with a phosphoramidite. In some embodiments, efficiency of deblocking in a phosphoramidite oligonucleic acid synthesis method contributes to error rate. In some cases, the substrates and/or synthesis methods described herein allow for removal of 5′-hydroxyl protecting groups at efficiencies greater than 98%, 98.5%, 99%, 99.5%, 99.6%, 99.7%, 99.8%, 99.9%, 99.95%, 99.96%, 99.97%, 99.98%, or 99.99%. In some embodiments, error rate is reduced by minimization of depurination side reactions.

Methods for oligonucleic acid synthesis, in various embodiments, include processes involving phosphoramidite chemistry. In some embodiments, oligonucleic acid synthesis comprises coupling a base with phosphoramidite. In some embodiments, oligonucleic acid synthesis comprises coupling a base by deposition of phosphoramidite under coupling conditions, wherein the same base is optionally deposited with phosphoramidite more than once, i.e. double coupling. In some embodiments, oligonucleic acid synthesis comprises capping of unreacted sites. In some cases, capping is optional. In some embodiments, oligonucleic acid synthesis comprises oxidation. In some embodiments, oligonucleic acid synthesis comprises deblocking or detritylation. In some embodiments, oligonucleic acid synthesis comprises sulfurization. In some cases, oligonucleic acid synthesis comprises either oxidation or sulfurization. In some embodiments, between one or each step during an oligonucleic acid synthesis reaction, the substrate is washed, for example, using tetrazole or acetonitrile. Time frames for any one step in a phosphoramidite synthesis method include less than about 2 min, 1 min, 50 sec, 40 sec, 30 sec, 20 sec and 10 sec.

Oligonucleic acid synthesis using a phosphoramidite method comprises the subsequent addition of a phosphoramidite building block (e.g., nucleoside phosphoramidite) to a growing oligonucleic acid chain for the formation of a phosphite triester linkage. Phosphoramidite oligonucleic acid synthesis proceeds in the 3′ to 5′ direction. Phosphoramidite oligonucleic acid synthesis allows for the controlled addition of one nucleotide to a growing nucleic acid chain per synthesis cycle. In some embodiments, each synthesis cycle comprises a coupling step. Phosphoramidite coupling involves the formation of a phosphite triester linkage between an activated nucleoside phosphoramidite and a nucleoside bound to the substrate, for example, via a linker. In some embodiments, the nucleoside phosphoramidite is provided to the substrate activated. In some embodiments, the nucleoside phosphoramidite is provided to the substrate with an activator. In some embodiments, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some embodiments, the addition of nucleoside phosphoramidite is performed in an anhydrous environment, for example, in anhydrous acetonitrile. Following addition of a nucleoside phosphoramidite, the substrate is optionally washed. In some embodiments, the coupling step is repeated one or more additional times, optionally with a wash step between nucleoside phosphoramidite additions to the substrate. In some embodiments, an oligonucleic acid synthesis method used herein comprises 1, 2, 3 or more sequential coupling steps. Prior to coupling, in many cases, the nucleoside bound to the substrate is de-protected by removal of a protecting group, where the protecting group functions to prevent polymerization. A common protecting group is 4,4′-dimethoxytrityl (DMT).

Following coupling, phosphoramidite oligonucleic acid synthesis methods optionally comprise a capping step. In a capping step, the growing oligonucleic acid is treated with a capping agent. A capping step generally serves to block unreacted substrate-bound 5′-OH groups after coupling from further chain elongation, preventing the formation of oligonucleic acids with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the oligonucleotide thus reducing the yield of the full-length product. The O6 modifications may be removed by treatment with the capping reagent prior to oxidation with I₂/water. In some embodiments, inclusion of a capping step during oligonucleic acid synthesis decreases the error rate as compared to synthesis without capping. As an example, the capping step comprises treating the substrate-bound oligonucleic acid with a mixture of acetic anhydride and 1-methylimidazole. Following a capping step, the substrate is optionally washed.

In some embodiments, following addition of a nucleoside phosphoramidite, and optionally after capping and one or more wash steps, the substrate bound growing nucleic acid is oxidized. The oxidation step comprises the phosphite triester is oxidized into a tetracoordinated phosphate triester, a protected precursor of the naturally occurring phosphate diester internucleoside linkage. In some cases, oxidation of the growing oligonucleic acid is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing oligonucleic acid is optionally washed. In some embodiments, the step of oxidation is substituted with a sulfurization step to obtain oligonucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the substrate bound growing oligonucleic acid must be removed so that the primary hydroxyl group can react with a next nucleoside phosphoramidite. In some embodiments, the protecting group is DMT and deblocking occurs with trichloroacetic acid in dichloromethane. Conducting detritylation for an extended time or with stronger than recommended solutions of acids may lead to increased depurination of solid support-bound oligonucleotide and thus reduces the yield of the desired full-length product. Methods and compositions of the invention described herein provide for controlled deblocking conditions limiting undesired depurination reactions. In some cases, the substrate bound oligonucleic acid is washed after deblocking. In some cases, efficient washing after deblocking contributes to synthesized oligonucleic acids having a low error rate.

Methods for the synthesis of oligonucleic acids typically involve an iterating sequence of the following steps: application of a protected monomer to an actively functionalized surface (e.g., locus) to link with either the activated surface, a linker or with a previously deprotected monomer; deprotection of the applied monomer so that it can react with a subsequently applied protected monomer; and application of another protected monomer for linking. One or more intermediate steps include oxidation or sulfurization. In some cases, one or more wash steps precede or follow one or all of the steps.

In some embodiments, oligonucleic acids are synthesized with photolabile protecting groups, where the hydroxyl groups generated on the surface are blocked by photolabile-protecting groups. When the surface is exposed to UV light, e.g., through a photolithographic mask, a pattern of free hydroxyl groups on the surface may be generated. These hydroxyl groups can react with photoprotected nucleoside phosphoramidites, according to phosphoramidite chemistry. A second photolithographic mask can be applied and the surface can be exposed to UV light to generate second pattern of hydroxyl groups, followed by coupling with 5′-photoprotected nucleoside phosphoramidite. Likewise, patterns can be generated and oligomer chains can be extended. Without being bound by theory, the lability of a photocleavable group depends on the wavelength and polarity of a solvent employed and the rate of photocleavage may be affected by the duration of exposure and the intensity of light. This method can leverage a number of factors, e.g., accuracy in alignment of the masks, efficiency of removal of photo-protecting groups, and the yields of the phosphoramidite coupling step. Further, unintended leakage of light into neighboring sites can be minimized. The density of synthesized oligomer per spot can be monitored by adjusting loading of the leader nucleoside on the surface of synthesis.

In some embodiments, the surface of the substrate that provides support for oligonucleic acid synthesis is chemically modified to allow for the synthesized oligonucleic acid chain to be cleaved from the surface. In some cases, the oligonucleic acid chain is cleaved at the same time as the oligonucleic acid is deprotected. In some cases, the oligonucleic acid chain is cleaved after the oligonucleic acid is deprotected. In an exemplary scheme, a trialkoxysilyl amine (e.g., (CH3CH2O)3Si—(CH2)2-NH2) is reacted with surface SiOH groups of a substrate, followed by reaction with succinic anhydride with the amine to create an amide linkage and a free OH on which the nucleic acid chain growth is supported.

Oligonucleic acids synthesized using the methods and substrates described herein, are optionally released from the surface from which they are synthesized. In some cases, oligonucleic acids are cleaved from the surface at this stage. Cleavage may include gas cleavage, e.g., with ammonia or methylamine. In some embodiments, all the loci in a single cluster collectively correspond to sequence encoding for a single gene, and, when cleaved, remain on the surface of the loci. In some embodiments, the application of ammonia gas simultaneous deprotects phosphates groups protected during the synthesis steps, i.e. removal of electron-withdrawing cyano group. In some embodiments, once released from the surface, oligonucleic acids are assembled into larger nucleic acids. Synthesized oligonucleic acids are useful, for example, as components for gene assembly/synthesis, site-directed mutagenesis, nucleic acid amplification, microarrays, and sequencing libraries.

In some embodiments, oligonucleic acids of predetermined sequence are designed to collectively span a large region of a target sequence, such as a gene. In some embodiments, larger oligonucleic acids are generated through ligation reactions to join the synthesized oligonucleic acids. One example of a ligation reaction is polymerase chain assembly (PCA). In some cases, at least of a portion of the oligonucleic acids are designed to include an appended region that is a substrate for universal primer binding. For PCA reactions, the presynthesized oligonucleic acids include overlaps with each other (e.g., 4, 20, 40 or more bases with overlapping sequence). During the polymerase cycles, the oligonucleic acids anneal to complementary fragments and then are filled in by polymerase. Each cycle thus increases the length of various fragments randomly depending on which oligonucleic acids find each other. Complementarity amongst the fragments allows for forming a complete large span of double-stranded DNA. In some cases, after the PCA reaction is complete, an error correction step is conducted using mismatch repair detecting enzymes to remove mismatches in the sequence. Once larger fragments of a target sequence are generated, they can be amplified. For example, in some cases, a target sequence comprising 5′ and 3′ terminal adapter sequences is amplified in a polymerase chain reaction (PCR) which includes modified primers, e.g., uracil containing primers the hybridize to the adapter sequences. The use of modified primers allows for removal of the primers through enzymatic reactions centered on targeting the modified base and/or gaps left by enzymes which cleave the modified base pair from the fragment. What remains is a double-stranded amplification product that lacks remnants of adapter sequence. In this way, multiple amplification products can be generated in parallel with the same set of primers to generate different fragments of double-stranded DNA.

In some embodiments, error correction is performed on synthesized oligonucleic acids and/or assembled products. An example strategy for error correction involves site-directed mutagenesis by overlap extension PCR to correct errors, which is optionally coupled with two or more rounds of cloning and sequencing. In certain embodiments, double-stranded nucleic acids with mismatches, bulges and small loops, chemically altered bases and/or other heteroduplexes are selectively removed from populations of correctly synthesized nucleic acids by affinity purification. In some embodiments, error correction is performed using proteins/enzymes that recognize and bind to or next to mismatched or unpaired bases within double-stranded nucleic acids to create a single or double-strand break or to initiate a strand transfer transposition event. Non-limiting examples of proteins/enzymes for error correction include endonucleases (T7 Endonuclease I, E. coli Endonuclease V, T4 Endonuclease VII, mung bean nuclease, Cell, E. coli Endonuclease IV, UVDE), restriction enzymes, glycosylases, ribonucleases, mismatch repair enzymes, resolvases, helicases, ligases, antibodies specific for mismatches, and their variants. Examples of specific error correction enzymes include T4 endonuclease 7, T7 endonuclease 1, S1, mung bean endonuclease, MutY, MutS, MutH, MutL, cleavase, CELI, and HINF1. In some cases, DNA mismatch-binding protein MutS (Thermus aquaticus) is used to remove failure products from a population of synthesized products. In some embodiments, error correction is performed using the enzyme Correctase. In some cases, error correction is performed using SURVEYOR endonuclease (Transgenomic), a mismatch-specific DNA endonuclease that scans for known and unknown mutations and polymorphisms for heteroduplex DNA.

Target Nucleic Acid Synthesis Systems

Provided herein, in some embodiments, are systems for the synthesis of oligonucleic acid libraries on a substrate. In some embodiments, the system comprises the substrate for synthesis support, as described elsewhere herein. In some embodiments, the system comprises a device for application of one or more reagents of a synthesis method, for example, an oligonucleic acid synthesizer. In some embodiments, the system comprises a device for treating the substrate with a fluid, for example, a flow cell. In some embodiments, the system comprises a device for moving the substrate between the application device and the treatment device.

In one aspect, provided is an automated system for use with an oligonucleic acid synthesis method described herein that is capable of processing one or more substrates, comprising: a material deposition device for spraying a microdroplet comprising a reagent on a substrate; a scanning transport for scanning the substrate adjacent to the material deposition device to selectively deposit the microdroplet at specified sites; a flow cell for treating the substrate on which the microdroplet is deposited by exposing the substrate to one or more selected fluids; an alignment unit for aligning the substrate correctly relative to the material deposition device each time when the substrate is positioned adjacent to the material deposition device for deposition. In some embodiments, the system optionally comprises a treating transport for moving the substrate between the material deposition device and the flow cell for treatment in the flow cell, where the treating transport and said scanning transport are different elements. In other embodiments, the system does not comprise a treating transport.

In some embodiments, a device for application of one or more reagents during a synthesis reagent is an oligonucleic acid synthesizer comprising a plurality of material deposition devices. In some embodiments, each material deposition device is configured to deposit nucleotide monomers, for example, for phosphoramidite synthesis. In some embodiments, the oligonucleic acid synthesizer deposits reagents to the resolved loci, wells, and/or microchannels of a substrate. In some cases, the oligonucleic acid synthesizer deposits a drop having a diameter less than about 200 um, 100 um, or 50 um in a volume less than about 1000, 500, 100, 50, or 20 pl. In some cases, the oligonucleic acid synthesizer deposits between about 1 and 10000, 1 and 5000, 100 and 5000, or 1000 and 5000 droplets per second. In some embodiments, the oligonucleic acid synthesizer uses organic solvents.

In some embodiments, during oligonucleic acid synthesis, the substrate is positioned within or sealed within a flow cell. In some embodiments, the flow cell provides continuous or discontinuous flow of liquids such as those comprising reagents necessary for reactions within the substrate, for example, oxidizers and/or solvents. In some embodiments, the flow cell provides continuous or discontinuous flow of a gas, such as nitrogen, for drying the substrate typically through enhanced evaporation of a volatile substrate. A variety of auxiliary devices are useful to improve drying and reduce residual moisture on the surface of the substrate. Examples of such auxiliary drying devices include, without limitation, a vacuum source, depressurizing pump and a vacuum tank. In some cases, an oligonucleic acid synthesis system comprises one or more flow cells, such as 2, 3, 4, 5, 6, 7, 8, 9, 10, or 20 and one or more substrates, such as 2, 3, 4, 5, 6, 7, 8, 9, 10 or 20. In some cases, a flow cell is configured to hold and provide reagents to the substrate during one or more steps in a synthesis reaction. In some embodiments, a flowcell comprises a lid that slides over the top of a substrate and can be clamped into place to form a pressure tight seal around the edge of the substrate. An adequate seal, includes, without limitation, a seal that allows for about 1, 2, 3, 4, 5, 6, 7, 8, 9 or 10 atmospheres of pressure. In some cases, the lid of the flow cell is opened to allow for access to an application device such as an oligonucleic acid synthesizer. In some cases, one or more steps of an oligonucleic acid synthesis method are performed on a substrate within a flow cell, without the transport of the substrate.

In some embodiments, during oligonucleic acid synthesis, a capping element, seals with the substrate, to form a resolved reactor. In some embodiments, a substrate having a plurality of clusters is configured to seal with a capping element having a plurality of caps, wherein when the substrate and capping element are sealed, each cluster is separate from another cluster to form separate resolved reactors for each cluster. In some instances, the capping element is not present in the system or is present and stationary. A resolved reactor is configured to allow for the transfer of fluid, including oligonucleic acids and/or reagents, from the substrate to the capping element and/or vice versa. In some embodiments, reactors are interconnected or in fluid communication. Fluid communication of reactors allows for washing and perfusion of new reagents for different steps of a synthesis reaction. In some cases, the resolved reactors comprise inlets and/or outlets. In some cases, the inlets and/or outlets are configured for use with a flow cell. As an example, a substrate is sealed within a flow cell where reagents can be introduced and flowed through the substrate, after which the reagents are collected. In some cases, the substrate is drained of fluid and purged with an inert gas such as nitrogen. The flow cell chamber can then be vacuum dried to reduce residual liquids or moisture to less than 1%, 0.1%, 0.01%, 0.001%, 0.0001%, or 0.00001% by volume of the chamber. In some embodiments, a vacuum chuck is in fluid communication with the substrate for removing gas.

In some embodiments, an oligonucleic acid synthesis system comprises one or more elements useful for downstream processing of the synthesized oligonucleic acids. As an example, the system comprises a temperature control element such as a thermal cycling device. In some embodiments, the temperature control element is used with a plurality of resolved reactors to perform nucleic acid assembly such as PCA and/or nucleic acid amplification such as PCR.

Substrates for Target Nucleic Acid Synthesis

In some embodiments, a substrate described herein comprises one or more features (e.g., wells, nanowells, channels, areas of active or passive functionalization) that provide support for a single molecule nucleic acid partitioned from a population of heterogeneous nucleic acids. In some cases, a substrate described herein comprises one or more features that provide support for performing an amplification reaction. As a non-limiting example, a substrate comprising a plurality of wells is suitable for receiving a plurality of partitioned single molecule fractions.

In some embodiments, a substrate described herein provides a surface for oligonucleic acid synthesis. In some embodiments, a substrate is configured for both active and passive functionalization of moieties bound to the surface at different areas of the substrate surface, generating distinct regions for oligonucleic acid synthesis to take place. In some embodiments, both active and passive functionalization agents are mixed within a particular region of the surface. Such a mixture provides a diluted region of active functionalization agent and therefore lowers the density of functionalization agent in a particular region.

In some embodiments, the surface comprises a high surface energy region. In one example, the high surface energy region is coated with amino silane. The silane group binds to the surface, while the rest of the molecule provides a distance from the surface and a free hydroxyl group at the end to which incoming bases attach. In some instances, the high surface energy region includes an active functionalization reagent, e.g., a chemical that binds the substrate efficiently and also couples efficiently to monomeric nucleic acid molecules. In some cases, such molecules have a hydroxyl group which is available for interacting with a nucleoside in a coupling reaction. In some embodiments, the amino silane is selected from the group consisting of 11-acetoxyundecyltriethoxysilane, n-decyltriethoxysilane, (3-aminopropyl)trimethoxysilane, (3-aminopropyl)triethoxysilane, (3-aminopropyl)triethoxysilane, glycidyloxypropyl/trimethoxysilane and N-(3-triethoxysilylpropyl)-4-hydroxybutyramide. In some instances the high surface energy region includes a passive functionalization reagent, e.g., a chemical that binds the substrate efficiently but does not couple efficiently to monomeric nucleic acid molecules.

In one aspect, described herein are substrates comprising a plurality of clusters, wherein each cluster comprises a plurality of loci that support the attachment and synthesis of oligonucleic acids. In one aspect, described herein are substrates comprising a plurality of clusters, wherein each cluster comprises a plurality of loci that support the amplification of single molecule fractions partitioned into the plurality of loci. In some embodiments, the term “locus” refers to a discrete region on a structure which provides support for oligonucleotides encoding for a single sequence to extend from the surface. In some embodiments, the term “locus” refers to a discrete region on a substructure which provides support for a partitioned nucleic acid molecule. In some embodiments, a locus is on a two dimensional surface, e.g., a substantially planar surface. In some embodiments, a locus is on a three-dimensional surface, e.g., a well, nanowell, channel, or post. In some embodiments, a surface of a locus comprises a material that is actively functionalized to attach to at least one nucleotide for oligonucleic acid synthesis, or preferably, a population of identical nucleotides for synthesis of a population of oligonucleic acids. In some embodiments, oligonucleic acid refers to a population of oligonucleic acids encoding for the same nucleic acid sequence. In some cases, a surface of a substrate is inclusive of one or a plurality of surfaces of a substrate.

In some embodiments, a substrate comprises a surface that supports the synthesis of a plurality of oligonucleic acids having different predetermined sequences at addressable locations on a common support. In some embodiments, a substrate provides support for the synthesis of more than 2,000; 5,000; 10,000; 20,000; 50,000; 100,000; 200,000; 400,000; 600,000; 800,000; 1,000,000; 1,500,000; 2,000,000; 2,500,000; 3,000,000; 3,500,000; 4,000,000; 4,500,000; 5,000,000; 10,000,000 or more non-identical oligonucleic acids. In some embodiments, at least a portion of the oligonucleic acids have an identical sequence or are configured to be synthesized with an identical sequence. In some embodiments, the substrate provides a surface environment for the growth of oligonucleic acids having at least 80, 90, 100, 120, 150, 175, 200, 225, 250, 275, 300, 325, 350, 375, 400, 425, 450, 475, 500 bases or more.

In some embodiments, oligonucleic acids are synthesized on distinct loci of a substrate, wherein each locus supports the synthesis of a population of oligonucleic acids. In some cases, each locus supports the synthesis of a population of oligonucleic acids having a different sequence than a population of oligonucleic acids grown on another locus. In some embodiments, the loci of a substrate are located within a plurality of clusters. In some instances, a substrate comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some embodiments, a substrate comprises more than 2,000; 5,000; 10,000; 100,000; 200,000; 300,000; 400,000; 500,000; 600,000; 700,000; 800,000; 900,000; 1,000,000; 2,000,000; 500,000; 800,000; 1,000,000; 2,000,000; 3,000,000; 4,000,000, 5,000,000, 10,000,000 or more distinct loci. The amount of loci within a single cluster is varied in different embodiments. In some cases, each cluster includes 1, 2, 3, 4, 5, 6, 7, 8, 9, 10, 20, 30, 40, 50, 60, 70, 80, 90, 100, 120, 130, 150 or more loci.

In some embodiments, the number of distinct oligonucleic acids synthesized on a substrate is dependent on the number of distinct loci available in the substrate. In some cases, a substrate comprises from about 10 loci per mm² to about 500 mm², from about 50 loci per mm² to about 500 mm², from about 100 loci per mm² to about 500 mm², from about 10 loci per mm² to about 250 mm², from about 50 loci per mm² to about 250 mm², from about 100 loci per mm² to about 200 mm², or from about 50 loci per mm² to about 200 mm². In some embodiments, the distance between the centers of two adjacent loci within a cluster is from about 10 um to about 500 um, from about 10 um to about 200 um, or from about 10 um to about 100 um.

In some embodiments, the number of distinct nucleic acids or genes assembled from a plurality of oligonucleic acids synthesized on a substrate is dependent on the number of clusters available in the substrate. In some embodiments, the density of clusters within a substrate is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some embodiments, a substrate comprises from about 1 cluster per 10 mm² to about 10 clusters per 1 mm². In some embodiments, the distance between the centers of two adjacent clusters is greater than about 50 um, 100 um, 200 um, 500 um, 1000 um, or 2000 um or 5000 um. In some cases, the distance between the centers of two adjacent clusters is less than about 2000 um, 1000 um, 500 um, 100 um or 50 um.

In various embodiments, a substrate comprises raised and/or lowered features. One benefit of having such features is an increase in surface area to support oligonucleic acid synthesis. In some embodiments, a substrate having raised and/or lowered features is referred to as a three-dimensional substrate. In some cases, a three-dimensional substrate comprises one or more channels. In some cases, one or more loci comprise a channel. In some cases, the channels are accessible to reagent deposition via a deposition device such as an oligonucleic acid synthesizer. In some cases, reagents and/or fluids may collect in a larger well in fluid communication one or more channels. For example, a substrate comprises a plurality of channels corresponding to a plurality of loci with a cluster, and the plurality of channels are in fluid communication with one well of the cluster. In some methods, a library of oligonucleic acids are synthesized in a plurality of loci of a cluster, followed by the assembly of the oligonucleic acids into a large nucleic acid such as gene, wherein the assembly of the large nucleic acid optionally occurs within a well of the cluster, e.g., by using PCA.

A well of a substrate may have the same or different width, height, and/or volume as another well of the substrate. A channel of a substrate may have the same or different width, height, and/or volume as another channel of the substrate. In some embodiments, the diameter of a cluster or the diameter of a well comprising a cluster, or both, is between about 0.05 mm to about 10 mm, between about 0.05 mm and about 5 mm, between about 0.05 mm and about 2 mm, between about 0.1 mm and 10 mm, between about 0.2 mm and 10 mm, between about 0.3 mm and about 10 mm, between about 0.4 mm and about 10 mm, between about 0.5 mm and 10 mm, between about 0.5 mm and about 5 mm, or between about 0.5 mm and about 2 mm. In some embodiments, the diameter of a cluster or well or both is between about 1.0 and 1.3 mm. In some embodiments, the diameter of a cluster or well or both is about 1.150 mm. The diameter of a cluster refers to clusters within a two-dimensional or three-dimensional substrate.

In some embodiments, the height of a well is from about 20 um to about 1000 um, from about 50 um to about 1000 um, from about 100 um to about 1000 um, from about 200 um to about 1000 um, from about 300 um to about 1000 um, from about 400 um to about 1000 um, or from about 500 um to about 1000 um. In some cases, the height of a well is less than about 1000 um, less than about 900 um, less than about 800 um, less than about 700 um, or less than about 600 um.

In some embodiments, a substrate comprises a plurality of channels corresponding to a plurality of loci within a cluster, wherein the height or depth of a channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some embodiments, the diameter of a channel, locus (e.g., in a substantially planar substrate) or both channel and locus (e.g., in a three-dimensional substrate wherein a locus corresponds to a channel) is from about 1 um to about 1000 um, from about 1 um to about 500 um, from about 1 um to about 200 um, from about 1 um to about 100 um, from about 5 um to about 100 um, or from about 10 um to about 100 um, for example, about 50 um.

The substrates provided may be fabricated from a variety of materials suitable for the methods and compositions described herein. In certain embodiments, substrate materials are fabricated to exhibit a low level of nucleotide binding. In some cases, substrate materials are modified to generate distinct surfaces that exhibit a high level of nucleotide binding. In some embodiments, substrate materials are transparent to visible and/or UV light. In some embodiments, substrate materials are sufficiently conductive, e.g., are able to form uniform electric fields across all or a portion of a substrate. In some embodiments, conductive materials may be connected to an electric ground. In some cases, the substrate is heat conductive or insulated. In some cases, the materials are chemical resistant and heat resistant to support chemical or biochemical reactions, for example oligonucleic acid synthesis reaction processes. In some embodiments, a substrate comprises flexible materials. Flexible materials include, without limitation, modified nylon, unmodified nylon, nitrocellulose, polypropylene, and the like. In some embodiments, a substrate comprises rigid materials. Rigid materials include, without limitation, glass, fuse silica, silicon, silicon dioxide, silicon nitride, plastics (for example, polytetrafluoroethylene, polypropylene, polystyrene, polycarbonate, and blends thereof, and the like), and metals (for example, gold, platinum, and the like). In some embodiments, a substrate is fabricated from a material comprising silicon, polystyrene, agarose, dextran, cellulosic polymers, polyacrylamides, polydimethylsiloxane (PDMS), glass, or any combination thereof. The substrates may be manufactured with a combination of materials listed herein or any other suitable material known in the art.

Surface Modifications

In various embodiments, surface modifications are employed for the chemical and/or physical alteration of a surface by an additive or subtractive process to change one or more chemical and/or physical properties of a substrate surface or a selected site or region of a substrate surface. For example, surface modification may involve (1) changing the wetting properties of a surface, (2) functionalizing a surface, i.e. providing, modifying or substituting surface functional groups, (3) defunctionalizing a surface, i.e. removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some cases, the addition of a chemical layer on top of a surface (referred to as adhesion promoter) facilitates structured patterning of loci on a surface of a substrate. Exemplary surfaces which can benefit from adhesion promotion include, without limitation, glass, silicon, silicon dioxide and silicon nitride. In some cases, the adhesion promoter is a chemical with a high surface energy. In some embodiments, a second chemical layer is deposited on a surface of a substrate. In some cases, the second chemical layer has a low surface energy. The surface energy of a chemical layer coated on a surface can facilitate localization of droplets on the surface. Depending on the patterning arrangement selected, the proximity of loci and/or area of fluid contact at the loci can be altered.

In some embodiments, a substrate surface is modified with one or more different layers of compounds. Such modification layers of interest include, without limitation, inorganic and organic layers such as metals, metal oxides, polymers, small organic molecules and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyetheyleneamines, polyarylene sulfides, polysiloxanes, polyimides, polyacetates, and any other suitable compounds described herein or otherwise known in the art. In some cases, polymers are heteropolymeric. In some cases, polymers are homopolymeric. In some cases, polymers comprise functional moieties or are conjugated.

In some embodiments, resolved loci of a substrate are functionalized with one or more moieties that increase and/or decrease surface energy. In some cases, a moiety is chemically inert. In some cases, a moiety is configured to support a desired chemical reaction, for example, one or more processes in an oligonucleic acid synthesis reaction. The surface energy, or hydrophobicity, of a surface is a factor for determining the affinity of a nucleotide to attach onto the surface. In some embodiments, a method for substrate functionalization comprises: (a) providing a substrate having a surface that comprises silicon dioxide; and (b) silanizing the surface using, a suitable silanizing agent described herein or otherwise known in the art, for example, an organofunctional alkoxysilane molecule. In some cases, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some embodiments, a substrate surface comprises functionalized with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluoroethylene. Other methods and functionalizing agents are described in U.S. Pat. No. 5,474,796, which is herein incorporated by reference in its entirety.

In some embodiments, a substrate surface is functionalized by contact with a derivatizing composition that contains a mixture of silanes, under reaction conditions effective to couple the silanes to the substrate surface, typically via reactive hydrophilic moieties present on the substrate surface. Silanization generally can be used to cover a surface through self-assembly with organofunctional alkoxysilane molecules. A variety of siloxane functionalizing reagents can further be used as currently known in the art, e.g., for lowering or increasing surface energy. The organofunctional alkoxysilanes are classified according to their organic functions. Non-limiting examples of siloxane functionalizing reagents include hydroxyalkyl siloxanes (silylate surface, functionalizing with diborane and oxidizing the alcohol by hydrogen peroxide), diol (dihydroxyalkyl) siloxanes (silylate surface, and hydrolyzing to diol), aminoalkyl siloxanes (amines require no intermediate functionalizing step), glycidoxysilanes (3-glycidoxypropyl-dimethyl-ethoxysilane, glycidoxy-trimethoxysilane), mercaptosilanes (3-mercaptopropyl-trimethoxysilane, 3-4 epoxycyclohexyl-ethyltrimethoxysilane or 3-mercaptopropyl-methyl-dimethoxysilane), bicyclohepthenyl-trichlorosilane, butyl-aldehydr-trimethoxysilane, or dimeric secondary aminoalkyl siloxanes. The hydroxyalkyl siloxanes can include allyl trichlorochlorosilane turning into 3-hydroxypropyl, or 7-oct-1-enyl trichlorochlorosilane turning into 8-hydroxyoctyl. The aminoalkyl siloxanes include 3-aminopropyl trimethoxysilane turning into 3-aminopropyl (3-aminopropyl-triethoxysilane, 3-aminopropyl-diethoxy-methylsilane, 3-aminopropyl-dimethyl-ethoxysilane, or 3-aminopropyl-trimethoxysilane). The dimeric secondary aminoalkyl siloxanes can be bis (3-trimethoxysilylpropyl) amine turning into bis(silyloxylpropyl)amine.

In some embodiments, the functionalizing agent comprises 11-acetoxyundecyltriethoxysilane, n-decyltriethoxysilane, (3-aminopropyl)trimethoxysilane, (3-aminopropyl)triethoxysilane, (3-aminopropyl)triethoxysilane, glycidyloxypropyl/trimethoxysilane and N-(3-triethoxysilylpropyl)-4-hydroxybutyramide.

In some embodiments, a substrate surface is contacting with a mixture of functionalization groups, e.g., amino silanes, which can be in any different ratio. In some embodiments, a mixture comprises at least 2, 3, 4, 5 or more different types of functionalization agents. In some embodiments, the mixture comprises 1, 2, 3 or more silanes. In some embodiments, desired surface tensions, wettabilities, water contact angles, and/or contact angles for other suitable solvents are achieved by providing a substrate surface with a suitable ratio of functionalization agents. In some cases, the agents in a mixture are chosen from suitable reactive and inert moieties, thus diluting the surface density of reactive groups to a desired level for downstream reactions. In some embodiments, the density of the fraction of a surface functional group that reacts to form a growing oligonucleotide in an oligonucleotide synthesis reaction is about 0.005 to about 100.0 μMol/m².

In some embodiments, a surface of a substrate is prepared to have a low surface energy. In some cases, a surface is functionalized to enable covalent binding of molecular moieties that can lower the surface energy so that wettability can be reduced. In some embodiments, a surface of a substrate is prepared to have a high surface energy and increased wettability.

In some instances, a surface is modified to have a higher surface energy, or become more hydrophilic with a coating of reactive hydrophilic moieties. By altering the surface energy of different parts of a substrate surface, spreading of a deposited reagent liquid (e.g., a reagent deposited during an oligonucleic acid synthesis method) can be adjusted, in some cases facilitated. In some embodiments, a droplet of reagent is deposited over a predetermined area of a surface with high surface energy. The liquid droplet can spread over and fill a small surface area having a higher surface energy as compared to a nearby surface. In some embodiments, a substrate surface is modified to comprise reactive hydrophilic moieties such as hydroxyl groups, carboxyl groups, thiol groups, and/or substituted or unsubstituted amino groups. Suitable materials include, but are not limited to, supports that can be used for solid phase chemical synthesis, e.g., cross-linked polymeric materials (e.g., divinylbenzene styrene-based polymers), agarose (e.g., Sepharose®), dextran (e.g., Sephadex®), cellulosic polymers, polyacrylamides, silica, glass (particularly controlled pore glass, or “CPG”), ceramics, and the like. The supports may be obtained commercially and used as is, or they may be treated or coated prior to functionalization.

The surface of the substrate or a portion of the surface of the substrate can be functionalized or modified to be more hydrophilic or hydrophobic as compared to the surface or the portion of the surface prior to the functionalization or modification. In some cases, one or more surfaces can be modified to have a difference in water contact angle of greater than 90°, 85°, 80°, 75°, 70°, 65°, 60°, 55°, 50°, 45°, 40°, 35°, 30°, 25°, 20°, 15° or 10° as measured on one or more uncurved, smooth or planar equivalent surfaces. Unless otherwise stated, water contact angles mentioned herein correspond to measurements that would be taken on uncurved, smooth or planar equivalents of the surfaces in question.

In some cases, hydrophilic resolved loci can be generated by first applying a protectant, or resist, over each locus within the substrate. The unprotected area can be then coated with a hydrophobic agent to yield an unreactive surface. For example, a hydrophobic coating can be created by chemical vapor deposition of (tridecafluorotetrahydrooctyl)-triethoxysilane onto the exposed oxide surrounding the protected circles. Finally, the protectant, or resist, can be removed exposing the loci regions of the substrate for further modification and oligonucleotide synthesis. In some embodiments, the initial modification of such unprotected regions may resist further modification and retain their surface functionalization, while newly unprotected areas can be subjected to subsequent modification steps.

Substrate Manufacture

In some embodiments, a method for functionalizing a surface of a substrate comprises photolithography. In various aspects, photolithography is a process for patterning substrates. In some examples, a photolithography method comprises 1) applying a photoresist to a substrate, 2) exposing the resist to light, e.g., using a binary mask opaque in some areas and clear in others, and 3) developing the resist; wherein the areas that were exposed are patterned. The patterned resist can then serve as a mask for subsequent processing steps, for example, etching, ion implantation, and deposition. After processing, the resist is typically removed, for example, by plasma stripping or wet chemical removal. In some embodiments, plasma descum is used to facilitate the removal of residual organic contaminants in resist cleared areas, for example, by using a typically short plasma cleaning step (e.g., oxygen plasma). In some embodiments, the resist is stripped by dissolving it in a suitable organic solvent, plasma etching, exposure and development, etc., thereby exposing the areas of the substrate that had been covered by the resist. In some embodiments, resist is removed in a process that does not remove functionalization groups or otherwise damage the functionalized surface.

In some embodiments, a method for functionalizing a surface of a substrate comprises a resist or photoresist coat. Photoresist, in many cases, refers to a light-sensitive material useful in photolithography to form patterned coatings. It is applied as a liquid to solidify on a substrate as volatile solvents in the mixture evaporate. In some embodiments, the resist is applied in a spin coating process as a thin film, e.g., 1 um to 100 um. In some cases, the coated resist is patterned by exposing it to light through a mask or reticle, changing its dissolution rate in a developer. In some cases, the resist cost is used as a sacrificial layer that serves as a blocking layer for subsequent steps that modify the underlying surface, e.g., etching, and then is removed by resist stripping. In some embodiments, the flow of resist throughout various features of the structure is controlled by the design of the structure. In some embodiments, a surface of a structure is functionalized while areas covered in resist are protected from active or passive functionalization.

In some cases, a preliminary step for surface functionalization is preparation of the surface. For example, the surface is chemically cleaned. In some embodiments, active functionalization is performed prior to lithography. In other embodiments, active functionalization is performed after lithography. In some embodiments, a substrate is prepared for oligonucleic acid synthesis by a process that comprises a first and a second functionalization step. For example, areas of a substrate functionalized by the first functionalization step block the deposition of functional groups in the second functionalization step. In some embodiments, differential functionalization facilitates spatial control of regions on a substrate where oligonucleic acids are synthesized. In some embodiments, differential functionalization provides improved flexibility to control the fluidic properties of the substrate. In some embodiments, after oligonucleic acid synthesis, oligonucleic acids are removed from the surface of a substrate and maintained in a reactor or optionally transferred to a second reactor device for assembly into a longer nucleic acid. In some cases, differential functionalization of the substrate improves the removal and/or transfer of a synthesized oligonucleic acid. In some embodiments, functionalized surfaces are relatively hydrophilic as compared to other surfaces of the substrate which are optionally relatively hydrophobic.

An exemplary workflow for the generation of differential functionalization patterns of a substrate is described herein. The following workflow is an example process and any step or component may be omitted or changed in accordance with properties desired of the final functionalized substrate. In some cases, additional components and/or process steps are added to the process workflows embodied herein. In some embodiments, a substrate is first cleaned, for example, using a piranha solution. An example of a cleaning process includes soaking a substrate in a piranha solution (e.g., 90% H₂SO₄, 10% H₂O₂) at an elevated temperature (e.g., 120° C.) and washing (e.g., water) and drying the substrate (e.g., nitrogen gas). The process optionally includes a post piranha treatment comprising soaking the piranha treated substrate in a basic solution (e.g., NH₄OH) followed by an aqueous wash (e.g., water). In some embodiments, a substrate is plasma cleaned, optionally following the piranha soak and optional post piranha treatment. An example of a plasma cleaning process comprises an oxygen plasma etch.

Active functionalization of a substrate involves the deposition of a molecule onto a surface of the substrate where the molecule enhances the substrates preferential binding for molecules deposited on the substrate surface. In some embodiments, the surface is deposited with an active functionalization agent following by vaporization. In some embodiments, the substrate is actively functionalized prior to cleaning, for example, by piranha treatment and/or plasma cleaning. In some embodiments, an active functionalization agent comprises N-(3-triethosysilylpropyl)-4-hydroxybutyramide. In various embodiments, an active functionalization agent comprises a silane. In some embodiments, an active functionalization agent comprises a solution of mixed silanes. The composition of the silanes in the mixed silane solution may be optimized depending on the surface of the substrate to be functionalized. In some cases, the density of oligonucleic acids (e.g., concentration) is altered to increase or reduce the amount of functionalization of the surface.

The process for substrate functionalization optionally comprises a resist coat and a resist strip. In some embodiments, following active surface functionalization, the substrate is spin coated with a resist, for example, SPR™ 3612 positive photoresist. The process for substrate functionalization, in various embodiments, comprises lithography with patterned functionalization. In some embodiments, photolithography is performed following resist coating. In some embodiments, after lithography, the substrate is visually inspected for lithography defects. The process for substrate functionalization, in some embodiments, comprises a descum step, whereby residues of the substrate are removed, for example, by plasma cleaning or etching. In some embodiments, the descum step is performed at some step after the lithography step.

The process for substrate functionalization, in some embodiments, comprises passive surface functionalization. In some embodiments, the surface is passively functionalized after active functionalization. In some embodiments, passive surface functionalization occurs after lithography. In some cases, the passive functionalization agent comprises a silane. In some cases, the passive functionalization agent comprises a mixture of silanes. In some cases, the passive functionalization agent comprises perfluorooctyltrichlorosilane.

In some embodiments, a substrate coated with a resist is treated to remove the resist, for example, after functionalization and/or after lithography. In some cases, the resist is removed with a solvent, for example, with a stripping solution comprising N-methyl-2-pyrrolidone. In some cases, resist stripping comprises sonication or ultrasonication. In some embodiments, a resist is coated and stripped, followed by active functionalization of the exposed areas to create a desired differential functionalization pattern.

In some embodiments, a substrate is functionalized by a process that comprises active functionalization as a step that follows resist coating and stripping. In some cases, the surface density of the active functionalized sites depends on the order in which the surface of the substrate is actively functionalized, e.g., whether the surface is actively functionalized prior to or after resist coating and stripping. For example, residues from the resist interfere with control of the surface density of the active sites. In some embodiments, a substrate is functionalized as a last step in substrate processing so that an active functionalization agent is deposited onto the substrate after any resist strip process. In this manner, residues from the resist may not interfere with the control of the surface density of the active sites.

In some cases, following oligonucleic acid synthesis using a substrate as a support, oligonucleic acids within one cluster are released from their respective surfaces and pool into the common well. In some embodiments, the pooled oligonucleic acids are assembled into a larger nucleic acid, such as a gene, within the well, so that the well functions as a reactor for nucleic acid assembly. In some embodiments, nucleic acid verification (e.g., sequencing of oligonucleic acids and/or assembled genes) is performed within a reactor or well. In some embodiments, one or more steps of a nucleic acid sorting method described herein is perform within a reactor or well. In some cases, a capping element or other device is placed over an open side of the well to create an enclosed reactor. A substrate comprising a well that functions as a reactor for each cluster has the advantage that each cluster may have a different environment from another cluster in another reactor. As an example, sealed reactors (e.g., those with capping elements) may experience controlled humidity, pressure or gas content.

Applications

Nucleic acids sorted using the cell-free methods described herein are suitable for use in various applications including, by way of example, hybridization methods such as gene expression analysis, genotyping by hybridization (competitive hybridization and heteroduplex analysis), sequencing by hybridization, probes for Southern blot analysis (labeled primers), probes for array (either microarray or filter array) hybridization, “padlock” probes usable with energy transfer dyes to detect hybridization in genotyping or expression assays, and other types of probes. The nucleic acids sorted in accordance with the this disclosure may also be used in enzyme-based reactions such as polymerase chain reaction (PCR), as primers for PCR, templates for PCR, allele-specific PCR (genotyping/haplotyping) techniques, real-time PCR, quantitative PCR, reverse transcriptase PCR, and other PCR techniques. The sorted nucleic acids may be used for various ligation techniques, including ligation-based genotyping, oligo ligation assays (OLA), ligation-based amplification, ligation of adapter sequences for cloning experiments, Sanger dideoxy sequencing (primers, labeled primers), high throughput sequencing (using electrophoretic separation or other separation method), primer extensions, mini-sequencings, and single base extensions (SBE). The nucleic acids sorted in accordance with this disclosure may be used in mutagenesis studies, (introducing a mutation into a known sequence with an oligo), reverse transcription (making a cDNA copy of an RNA transcript), gene synthesis, introduction of restriction sites (a form of mutagenesis), protein-DNA binding studies, and like experiments.

Computer Systems

Any of the systems described herein, may be operably linked to a computer and may be automated through a computer either locally or remotely. In various embodiments, the methods and systems of the invention may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. The computer systems may be programmed to interface between the user specified base sequence and the position of a material deposition device to deliver the correct reagents to specified regions of the substrate.

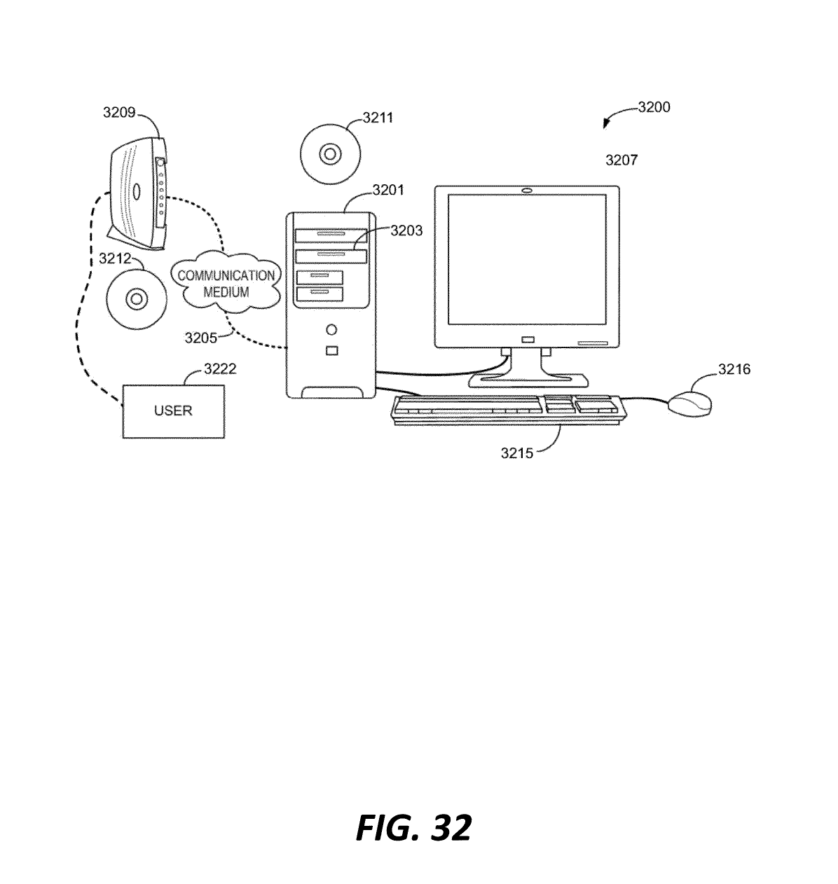

The computer system 3200 illustrated in FIG. 32 may be understood as a logical apparatus that can read instructions from media 3211 and/or a network port 3205, which can optionally be connected to server 3209 having fixed media 3212. The system, such as shown in FIG. 32 can include a CPU 3201, disk drives 3203, optional input devices such as keyboard 3215 and/or mouse 3216 and optional monitor 3207. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 3222 as illustrated in FIG. 32.

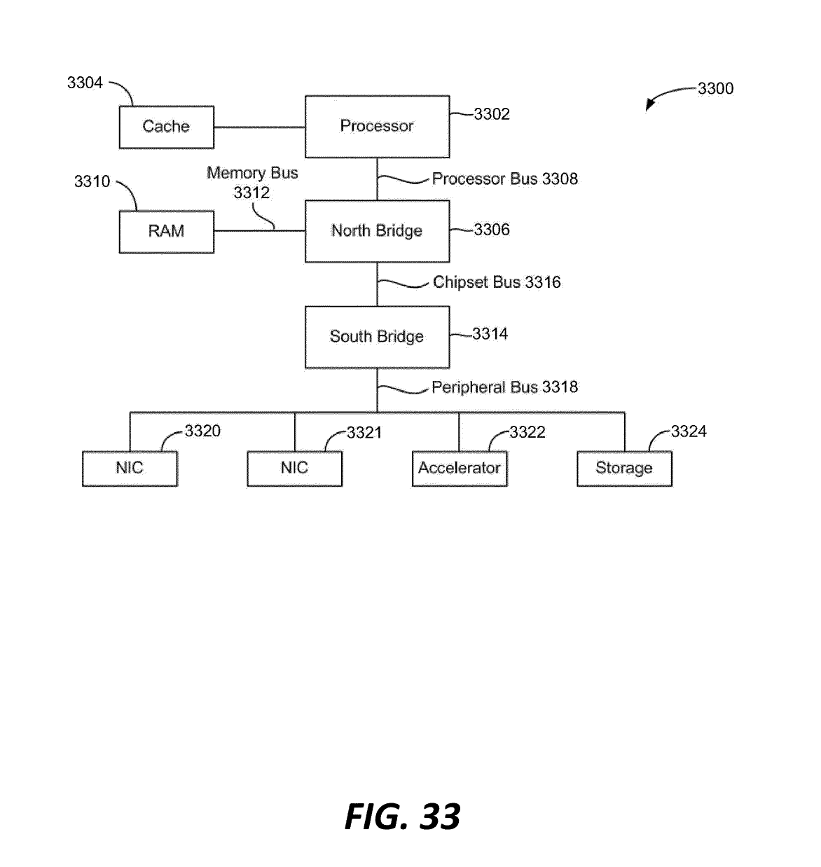

FIG. 33 is a block diagram illustrating a first example architecture of a computer system 3300 that can be used in connection with example embodiments of the present invention. As depicted in FIG. 33, the example computer system can include a processor 3302 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some embodiments, multiple processors or processors with multiple cores can also be used, whether in a single computer system, in a cluster, or distributed across systems over a network comprising a plurality of computers, cell phones, and/or personal data assistant devices.

As illustrated in FIG. 33, a high speed cache 3304 can be connected to, or incorporated in, the processor 3302 to provide a high speed memory for instructions or data that have been recently, or are frequently, used by processor 3302. The processor 3302 is connected to a north bridge 3306 by a processor bus 3308. The north bridge 3306 is connected to random access memory (RAM) 3310 by a memory bus 3312 and manages access to the RAM 3310 by the processor 3302. The north bridge 3306 is also connected to a south bridge 3314 by a chipset bus 3316. The south bridge 3314 is, in turn, connected to a peripheral bus 3318. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 3318. In some alternative architectures, the functionality of the north bridge can be incorporated into the processor instead of using a separate north bridge chip.

In some embodiments, system 2000 can include an accelerator card 2022 attached to the peripheral bus 2018. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 3324 and can be loaded into RAM 3310 and/or cache 3304 for use by the processor. The system 3300 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention. In this example, system 3300 also includes network interface cards (NICs) 3320 and 3321 connected to the peripheral bus for providing network interfaces to external storage, such as Network Attached Storage (NAS) and other computer systems that can be used for distributed parallel processing.

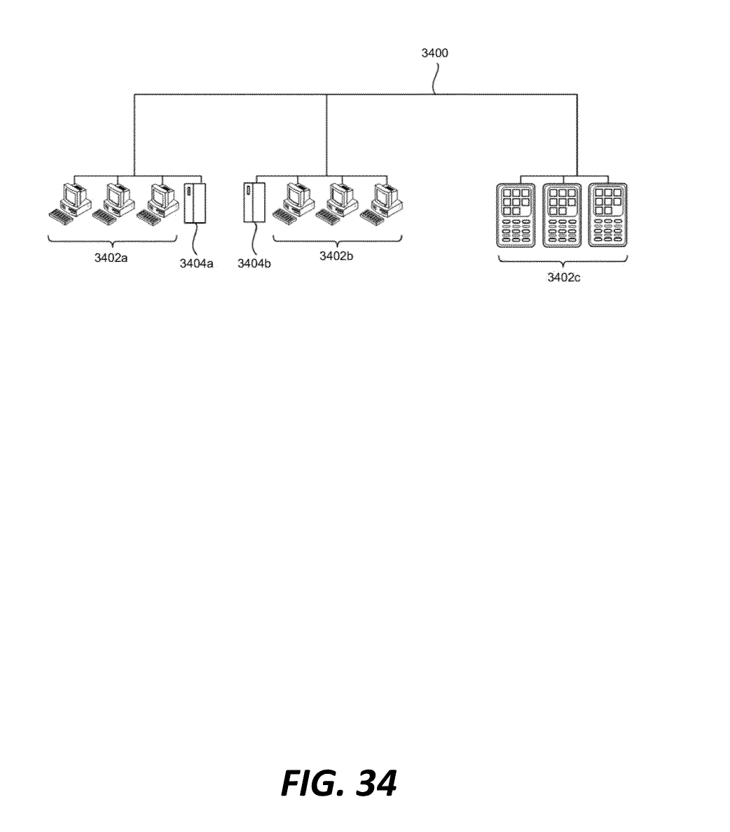

FIG. 34 is a diagram showing a network 3400 with a plurality of computer systems 3402a, and 3402b, a plurality of cell phones and personal data assistants 3402c, and Network Attached Storage (NAS) 3404a, and 3404b. In example embodiments, systems 3402a, 3402b, and 3402c can manage data storage and optimize data access for data stored in Network Attached Storage (NAS) 3404a and 3404b. A mathematical model can be used for the data and be evaluated using distributed parallel processing across computer systems 3402a, and 3402b, and cell phone and personal data assistant systems 3402c. Computer systems 3402a, and 3402b, and cell phone and personal data assistant systems 3402c can also provide parallel processing for adaptive data restructuring of the data stored in Network Attached Storage (NAS) 3404a and 3404b. FIG. 34 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space.

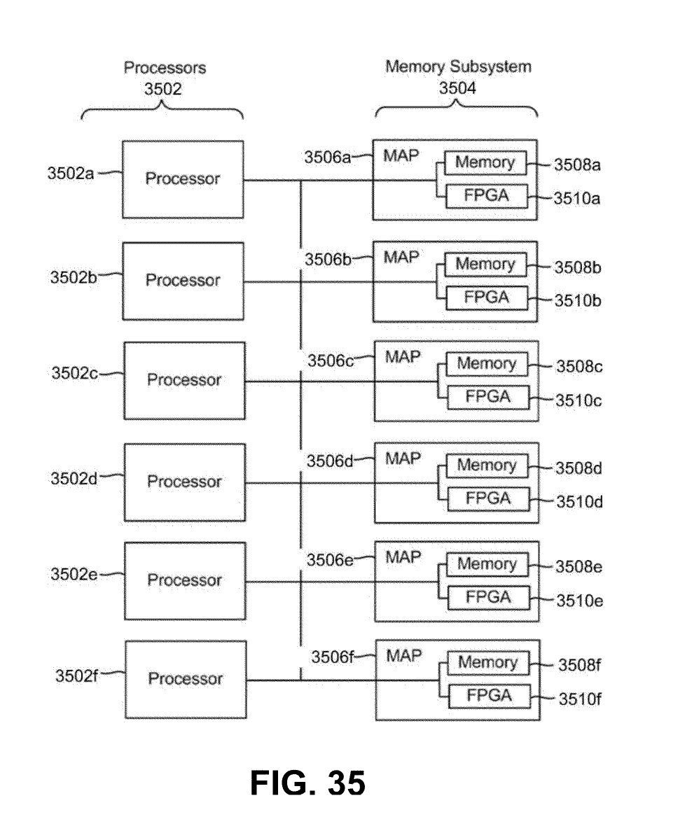

FIG. 35 is a block diagram of a multiprocessor computer system 3500 using a shared virtual address memory space in accordance with an example embodiment. The system includes a plurality of processors 3502a-f that can access a shared memory subsystem 3504. The system incorporates a plurality of programmable hardware memory algorithm processors (MAPs) 3506a-f in the memory subsystem 3504. Each MAP 3506a-f can comprise a memory 3508a-f and one or more field programmable gate arrays (FPGAs) 3510a-f. The MAP provides a configurable functional unit and particular algorithms or portions of algorithms can be provided to the FPGAs 3510a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 3508a-f, allowing it to execute tasks independently of, and asynchronously from, the respective microprocessor 3502a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs) as referenced in FIG. 35, system on chips (SOCs), application specific integrated circuits (ASICs), or other processing and logic elements. For example, the Set Processor and Optimizer can be implemented with hardware acceleration through the use of a hardware accelerator card, such as accelerator card 3222 illustrated in FIG. 32.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1

### Synthesis of a 100-Mer Oligonucleic Acid on a Substantially Planar Substrate

A substantially planar substrate functionalized for oligonucleic acid synthesis was assembled into a flow cell and connected to an Applied Biosystems ABI394 DNA Synthesizer. In one experiment, the substrate was uniformly functionalized with N-(3-triethoxysilylpropyl)-4-hydroxybutyramide. In another experiment, the substrate was functionalized with a 5/95 mix of 11-acetoxyundecyltriethoxysilane and N-decyltriethoxysilane. Synthesis of 100-mer oligonucleic acids (“100-mer oligonucleotide”; 5′CGGGATCCTTATCGTCATCGTCGTACAGATCCCGACCCATTTGCTGTCCACCAGTC ATGCTAGCCATACCATGATGATGATGATGATGAGAACCCCGCAT##TTTTTTTTTT3′ (SEQ ID NO.: 1), where # denotes Thymidine-succinyl hexamide CED phosphoramidite (CLP-2244 from ChemGenes)) were performed using the methods of Table 1.

TABLE 1
Table 1. Method for oligonucleic acid synthesis.

| General DNA Synthesis | | |
|---|---|---|
| Process Name | Process Step | Time (sec) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

Synthesized 100-mer oligonucleic acids were extracted from the substrate surface and analyzed on a Bioanalyzer chip (Agilent). The synthesized 100-mer oligonucleic acids were PCR amplified, cloned and Sanger sequenced. Table 2 summarizes the Sanger sequencing results for samples taken from spots 1-5 from one chip and spots 6-10 from a second chip.

TABLE 2
Table 2. Error rates of 100-mer oligonucleic acids
synthesized as determined by Sanger sequencing.

| Spot | Error rate | Cycle efficiency |
|---|---|---|
| 1 | 1/763 bp | 99.87% |
| 2 | 1/824 bp | 99.88% |
| 3 | 1/780 bp | 99.87% |
| 4 | 1/429 bp | 99.77% |
| 5 | 1/1525 bp | 99.93% |
| 6 | 1/1615 bp | 99.94% |
| 7 | 1/531 bp | 99.81% |
| 8 | 1/1769 bp | 99.94% |
| 9 | 1/854 bp | 99.88% |
| 10 | 1/1451 bp | 99.93% |

Overall, 89% (233/262) of the 100-mers that were sequenced had sequences without errors. Table 3 summarizes key error characteristics for the sequences obtained from the oligonucleic acid samples from spots 1-10.

TABLE 3
Table 3. Summary of error characteristics for sequences obtained from
synthesized 100-mer oligonucleic acid samples.

| | Sample ID/Spot No. | | | | |
|---|---|---|---|---|---|
| | OSA_0046/1 | OSA_0047/2 | OSA_0048/3 | OSA_0049/4 | OSA_0050/5 |
| Total | 32 | 32 | 32 | 32 | 32 |
| Sequences | | | | | |
| Sequencing | 25 of 28 | 27 of 27 | 26 of 30 | 21 of 23 | 25 of 26 |
| Quality | | | | | |
| Oligo | 23 of 25 | 25 of 27 | 22 of 26 | 18 of 21 | 24 of 25 |
| Quality | | | | | |
| ROI | 2500 | 2698 | 2561 | 2122 | 2499 |
| Match | | | | | |
| Count | | | | | |
| ROI | 2 | 2 | 1 | 3 | 1 |
| Mutation | | | | | |
| ROI | 0 | 0 | 0 | 0 | 0 |
| Multi | | | | | |
| Base | | | | | |
| Deletion | | | | | |
| ROI | 1 | 0 | 0 | 0 | 0 |
| Small | | | | | |
| Insertion | | | | | |
| ROI | 0 | 0 | 0 | 0 | 0 |
| Single | | | | | |
| Base | | | | | |
| Deletion | | | | | |
| Large | 0 | 0 | 1 | 0 | 0 |
| Deletion | | | | | |
| Count | | | | | |
| Mutation: | 2 | 2 | 1 | 2 | 1 |
| G > A | | | | | |
| Mutation: | 0 | 0 | 0 | 1 | 0 |
| T > C | | | | | |
| ROI | 3 | 2 | 2 | 3 | 1 |
| Error | | | | | |
| Count | | | | | |
| ROI | Err: ~1 | Err: ~1 | Err: ~1 | Err: ~1 | Err: ~1 |
| Error | in 834 | in 1350 | in 1282 | in 708 | in 2500 |
| Rate | | | | | |
| ROI | MP Err: | MP Err: | MP Err: | MP Err: | MP Err: |
| Minus | ~1 in | ~1 in | ~1 in | ~1 in | ~1 in |
| Primer | 763 | 824 | 780 | 429 | 1525 |
| Error | | | | | |
| Rate | | | | | |
| | Sample ID/Spot No. | | | | |
| | OSA_0051/6 | OSA_0052/7 | OSA_0053/8 | OSA_0054/9 | OSA_0055/10 |
| Total | 32 | 32 | 32 | 32 | 32 |
| Sequences | | | | | |
| Sequencing | 29 of 30 | 27 of 31 | 29 of 31 | 28 of 29 | 25 of 28 |
| Quality | | | | | |
| Oligo | 25 of 29 | 22 of 27 | 28 of 29 | 26 of 28 | 20 of 25 |
| Quality | | | | | |
| ROI | 2666 | 2625 | 2899 | 2798 | 2348 |
| Match | | | | | |
| Count | | | | | |
| ROI | 0 | 2 | 1 | 2 | 1 |
| Mutation | | | | | |
| ROI | 0 | 0 | 0 | 0 | 0 |
| Multi | | | | | |
| Base | | | | | |
| Deletion | | | | | |
| ROI | 0 | 0 | 0 | 0 | 0 |
| Small | | | | | |
| Insertion | | | | | |
| ROI | 0 | 0 | 0 | 0 | 0 |
| Single | | | | | |
| Base | | | | | |
| Deletion | | | | | |
| Large | 1 | 1 | 0 | 0 | 0 |
| Deletion | | | | | |
| Count | | | | | |
| Mutation: | 0 | 2 | 1 | 2 | 1 |
| G > A | | | | | |
| Mutation: | 0 | 0 | 0 | 0 | 0 |
| T > C | | | | | |
| ROI | 1 | 3 | 1 | 2 | 1 |
| Error | | | | | |
| Count | | | | | |
| ROI | Err: ~1 | Err: ~1 | Err: ~1 | Err: ~1 | Err: ~1 |
| Error | in 2667 | in 876 | in 2900 | in 1400 | in 2349 |
| Rate | | | | | |
| ROI | MP Err: | MP Err: | MP Err: | MP Err: | MP Err: |
| Minus | ~1 in | ~1 in | ~1 in | ~1 in | ~1 in |
| Primer | 1615 | 531 | 1769 | 854 | 1451 |
| Error | | | | | |
| Rate | | | | | |

### Example 2

### Gene Assembly in Reactors Using PCA

Gene assembly within nanoreactors created using a three-dimensional substrate was performed. PCA reactions were performed using oligonucleic acids described in Table 4 (SEQ ID NOs: 2-61) to assemble the 3075 base LacZ gene (SEQ ID NO.: 62) using the reaction mixture of Table 5 within individual nanoreactors.

TABLE 4
Oligonucleic acid sequences
(Sequence ID NOs.: 2-61) for
generating an assembled LacZ gene
product (SEQ ID NO.: 62) by PCA.

| Sequence Name | Sequence |
|---|---|
| Oligo 1, | 5′ATGACCATGATTACGGATTCACTGGC |
| SEQ ID NO.: 2 | CGTCGTTTTACAACGTCGTGACTGGGAA |
| | AACCCTGG3′ |
| Oligo 2, | 5′GCCAGCTGGCGAAAGGGGGATGTGCT |
| SEQ ID NO.: 3 | GCAAGGCGATTAAGTTGGGTAACGCCAG |
| | GGTTTTCCCAGTCACGAC3′ |
| Oligo 3, | 5′CCCCCTTTCGCCAGCTGGCGTAATAG |
| SEQ ID NO.: 4 | CGAAGAGGCCCGCACCGATCGCCCTTCC |
| | CAACAGTTGCGCAGCC3′ |
| Oligo 4, | 5′CGGCACCGCTTCTGGTGCCGGAAACC |
| SEQ ID NO.: 5 | AGGCAAAGCGCCATTCGCCATTCAGGCT |
| | GCGCAACTGTTGGGA3′ |
| Oligo 5, | 5′CACCAGAAGCGGTGCCGGAAAGCTGG |
| SEQ ID NO.: 6 | CTGGAGTGCGATCTTCCTGAGGCCGATA |
| | CTGTCGTCGTCCCCTC3′ |
| Oligo 6, | 5′GATAGGTCACGTTGGTGTAGATGGGC |
| SEQ ID NO.: 7 | GCATCGTAACCGTGCATCTGCCAGTTTG |
| | AGGGGACGACGACAGTATCGG3′ |
| Oligo 7, | 5′CCCATCTACACCAACGTGACCTATCC |
| SEQ ID NO.: 8 | CATTACGGTCAATCCGCCGTTTGTTCCC |
| | ACGGAGAATCCGACGGGTTG3′ |
| Oligo 8, | 5′GTCTGGCCTTCCTGTAGCCAGCTTTC |
| SEQ ID NO.: 9 | ATCAACATTAAATGTGAGCGAGTAACAA |
| | CCCGTCGGATTCTCCGTG3′ |
| Oligo 9, | 5′GCTGGCTACAGGAAGGCCAGACGCGA |
| SEQ ID NO.: 10 | ATTATTTTTGATGGCGTTAACTCGGCGT |
| | TTCATCTGTGGTGCAACGG3′ |
| Oligo 10, | 5′CAGGTCAAATTCAGACGGCAAACGAC |
| SEQ ID NO.: 11 | TGTCCTGGCCGTAACCGACCCAGCGCCC |
| | GTTGCACCACAGATGAAACG3′ |
| Oligo 11, | 5′CGTTTGCCGTCTGAATTTGACCTGAG |
| SEQ ID NO.: 12 | CGCATTTTTACGCGCCGGAGAAAACCGC |
| | CTCGCGGTGATGGTGCTG3′ |
| Oligo 12, | 5′GCCGCTCATCCGCCACATATCCTGAT |
| SEQ ID NO.: 13 | CTTCCAGATAACTGCCGTCACTCCAGCG |
| | CAGCACCATCACCGCGAG3′ |
| Oligo 13, | 5′AGGATATGTGGCGGATGAGCGGCATT |
| SEQ ID NO.: 14 | TTCCGTGACGTCTCGTTGCTGCATAAAC |
| | CGACTACACAAATCAGCGATTTC3′ |
| Oligo 14, | 5′CTCCAGTACAGCGCGGCTGAAATCAT |
| SEQ ID NO.: 15 | CATTAAAGCGAGTGGCAACATGGAAATC |
| | GCTGATTTGTGTAGTCGGTTTATG3′ |
| Oligo 15, | 5′ATTTCAGCCGCGCTGTACTGGAGGCT |
| SEQ ID NO.: 16 | GAAGTTCAGATGTGCGGCGAGTTGCGTG |
| | ACTACCTACGGGTAACAGTTT3′ |
| Oligo 16, | 5′AAAGGCGCGGTGCCGCTGGCGACCTG |
| SEQ ID NO.: 17 | CGTTTCACCCTGCCATAAAGAAACTGTT |
| | ACCCGTAGGTAGTCACG3′ |
| Oligo 17, | 5′GCGGCACCGCGCCTTTCGGCGGTGAA |
| SEQ ID NO.: 18 | ATTATCGATGAGCGTGGTGGTTATGCCG |
| | ATCGCGTCACACTACG3′ |
| Oligo 18, | 5′GATAGAGATTCGGGATTTCGGCGCTC |
| SEQ ID NO.: 19 | CACAGTTTCGGGTTTTCGACGTTCAGAC |
| | GTAGTGTGACGCGATCGGCA3′ |
| Oligo 19, | 5′GAGCGCCGAAATCCCGAATCTCTATC |
| SEQ ID NO.: 20 | GTGCGGTGGTTGAACTGCACACCGCCGA |
| | CGGCACGCTGATTGAAGCAG3′ |
| Oligo 20, | 5′CAGCAGCAGACCATTTTCAATCCGCA |
| SEQ ID NO.: 21 | CCTCGCGGAAACCGACATCGCAGGCTTC |
| | TGCTTCAATCAGCGTGCCG3′ |
| Oligo 21, | 5′CGGATTGAAAATGGTCTGCTGCTGCT |
| SEQ ID NO.: 22 | GAACGGCAAGCCGTTGCTGATTCGAGGC |
| | GTTAACCGTCACGAGCATCA3′ |
| Oligo 22, | 5′GCAGGATATCCTGCACCATCGTCTGC |
| SEQ ID NO.: 23 | TCATCCATGACCTGACCATGCAGAGGAT |
| | GATGCTCGTGACGGTTAACGC3′ |
| Oligo 23, | 5′CAGACGATGGTGCAGGATATCCTGCT |
| SEQ ID NO.: 24 | GATGAAGCAGAACAACTTTAACGCCGTG |
| | CGCTGTTCGCATTATCCGAAC3′ |
| Oligo 24, | 5′TCCACCACATACAGGCCGTAGCGGTC |
| SEQ ID NO.: 25 | GCACAGCGTGTACCACAGCGGATGGTTC |
| | GGATAATGCGAACAGCGCAC3′ |
| Oligo 25, | 5′GCTACGGCCTGTATGTGGTGGATGAA |
| SEQ ID NO.: 26 | GCCAATATTGAAACCCACGGCATGGTGC |
| | CAATGAATCGTCTGACCGATG3′ |
| Oligo 26, | 5′GCACCATTCGCGTTACGCGTTCGCTC |
| SEQ ID NO.: 27 | ATCGCCGGTAGCCAGCGCGGATCATCGG |
| | TCAGACGATTCATTGGCAC3′ |
| Oligo 27, | 5′CGCGTAACGCGAATGGTGCAGCGCGA |
| SEQ ID NO.: 28 | TCGTAATCACCCGAGTGTGATCATCTGG |
| | TCGCTGGGGAATGAATCAG3′ |
| Oligo 28, | 5′GGATCGACAGATTTGATCCAGCGATA |
| SEQ ID NO.: 29 | CAGCGCGTCGTGATTAGCGCCGTGGCCT |
| | GATTCATTCCCCAGCGACCAGATG3′ |
| Oligo 29, | 5′GTATCGCTGGATCAAATCTGTCGATC |
| SEQ ID NO.: 30 | CTTCCCGCCCGGTGCAGTATGAAGGCGG |
| | CGGAGCCGACACCACGGC3′ |
| Oligo 30, | 5′CGGGAAGGGCTGGTCTTCATCCACGC |
| SEQ ID NO.: 31 | GCGCGTACATCGGGCAAATAATATCGGT |
| | GGCCGTGGTGTCGGCTC3′ |
| Oligo 31, | 5′TGGATGAAGACCAGCCCTTCCCGGCT |
| SEQ ID NO.: 32 | GTGCCGAAATGGTCCATCAAAAAATGGC |
| | TTTCGCTACCTGGAGAGAC3′ |
| Oligo 32, | 5′CCAAGACTGTTACCCATCGCGTGGGC |
| SEQ ID NO.: 33 | GTATTCGCAAAGGATCAGCGGGCGCGTC |
| | TCTCCAGGTAGCGAAAGCC3′ |
| Oligo 33, | 5′CGCGATGGGTAACAGTCTTGGCGGTT |
| SEQ ID NO.: 34 | TCGCTAAATACTGGCAGGCGTTTCGTCA |
| | GTATCCCCGTTTACAGGGC3′ |
| Oligo 34, | 5′GCCGTTTTCATCATATTTAATCAGCG |
| SEQ ID NO.: 35 | ACTGATCCACCCAGTCCCAGACGAAGCC |
| | GCCCTGTAAACGGGGATACTGACG3′ |
| Oligo 35, | 5′CAGTCGCTGATTAAATATGATGAAAA |
| SEQ ID NO.: 36 | CGGCAACCCGTGGTCGGCTTACGGCGGT |
| | GATTTTGGCGATACGCCGAACG3′ |
| Oligo 36, | 5′GCGGCGTGCGGTCGGCAAAGACCAGA |
| SEQ ID NO.: 37 | CCGTTCATACAGAACTGGCGATCGTTCG |
| | GCGTATCGCCAAA3′ |
| Oligo 37, | 5′CGACCGCACGCCGCATCCAGCGCTGA |
| SEQ ID NO.: 38 | CGGAAGCAAAACACCAGCAGCAGTTTTT |
| | CCAGTTCCGTTTATCCG3′ |
| Oligo 38, | 5′CTCGTTATCGCTATGACGGAACAGGT |
| SEQ ID NO.: 39 | ATTCGCTGGTCACTTCGATGGTTTGCCC |
| | GGATAAACGGAACTGGAAAAACTGC3′ |
| Oligo 39, | 5′AATACCTGTTCCGTCATAGCGATAAC |
| SEQ ID NO.: 40 | GAGCTCCTGCACTGGATGGTGGCGCTGG |
| | ATGGTAAGCCGCTGGCAAGCG3′ |
| Oligo 40, | 5′GTTCAGGCAGTTCAATCAACTGTTTA |
| SEQ ID NO.: 41 | CCTTGTGGAGCGACATCCAGAGGCACTT |
| | CACCGCTTGCCAGCGGCTTACC3′ |
| Oligo 41, | 5′CAAGGTAAACAGTTGATTGAACTGCC |
| SEQ ID NO.: 42 | TGAACTACCGCAGCCGGAGAGCGCCGGG |
| | CAACTCTGGCTCACAGTACGCGTA3′ |
| Oligo 42, | 5′GCGCTGATGTGCCCGGCTTCTGACCA |
| SEQ ID NO.: 43 | TGCGGTCGCGTTCGGTTGCACTACGCGT |
| | ACTGTGAGCCAGAGTTG3′ |
| Oligo 43, | 5′CCGGGCACATCAGCGCCTGGCAGCAG |
| SEQ ID NO.: 44 | TGGCGTCTGGCGGAAAACCTCAGTGTGA |
| | CGCTCCCCGCCGC3′ |
| Oligo 44, | 5′CCAGCTCGATGCAAAAATCCATTTCG |
| SEQ ID NO.: 45 | CTGGTGGTCAGATGCGGGATGGCGTGGG |
| | ACGCGGCGGGGAGCGTC3′ |
| Oligo 45, | 5′CGAAATGGATTTTTGCATCGAGCTGG |
| SEQ ID NO.: 46 | GTAATAAGCGTTGGCAATTTAACCGCCA |
| | GTCAGGCTTTCTTTCACAGATGTG3′ |
| Oligo 46, | 5′TGAACTGATCGCGCAGCGGCGTCAGC |
| SEQ ID NO.: 47 | AGTTGTTTTTTATCGCCAATCCACATCT |
| | GTGAAAGAAAGCCTGACTGG3′ |
| Oligo 47, | 5′GCCGCTGCGCGATCAGTTCACCCGTG |
| SEQ ID NO.: 48 | CACCGCTGGATAACGACATTGGCGTAAG |
| | TGAAGCGACCCGCATTGAC3′ |
| Oligo 48, | 5′GGCCTGGTAATGGCCCGCCGCCTTCC |
| SEQ ID NO.: 49 | AGCGTTCGACCCAGGCGTTAGGGTCAAT |
| | GCGGGTCGCTTCACTTA3′ |
| Oligo 49, | 5′CGGGCCATTACCAGGCCGAAGCAGCG |
| SEQ ID NO.: 50 | TTGTTGCAGTGCACGGCAGATACACTTG |
| | CTGATGCGGTGCTGAT3′ |
| Oligo 50, | 5′TCCGGCTGATAAATAAGGTTTTCCCC |
| SEQ ID NO.: 51 | TGATGCTGCCACGCGTGAGCGGTCGTAA |
| | TCAGCACCGCATCAGCAAGTG3′ |
| Oligo 51, | 5′GGGGAAAACCTTATTTATCAGCCGGA |
| SEQ ID NO.: 52 | AAACCTACCGGATTGATGGTAGTGGTCA |
| | AATGGCGATTACCGTTGATGTTGA3′ |
| Oligo 52, | 5′GGCAGTTCAGGCCAATCCGCGCCGGA |
| SEQ ID NO.: 53 | TGCGGTGTATCGCTCGCCACTTCAACAT |
| | CAACGGTAATCGCCATTTGAC3′ |
| Oligo 53, | 5′GCGGATTGGCCTGAACTGCCAGCTGG |
| SEQ ID NO.: 54 | CGCAGGTAGCAGAGCGGGTAAACTGGCT |
| | CGGATTAGGGCCGCAAG3′ |
| Oligo 54, | 5′GGCAGATCCCAGCGGTCAAAACAGGC |
| SEQ ID NO.: 55 | GGCAGTAAGGCGGTCGGGATAGTTTTCT |
| | TGCGGCCCTAATCCGAGC3′ |
| Oligo 55, | 5′GTTTTGACCGCTGGGATCTGCCATTG |
| SEQ ID NO.: 56 | TCAGACATGTATACCCCGTACGTCTTCC |
| | CGAGCGAAAACGGTCTGC3′ |
| Oligo 56, | 5′GTCGCCGCGCCACTGGTGTGGGCCAT |
| SEQ ID NO.: 57 | AATTCAATTCGCGCGTCCCGCAGCGCAG |
| | ACCGTTTTCGCTCGG3′ |
| Oligo 57, | 5′ACCAGTGGCGCGGCGACTTCCAGTTC |
| SEQ ID NO.: 58 | AACATCAGCCGCTACAGTCAACAGCAAC |
| | TGATGGAAACCAGCCATC3′ |
| Oligo 58, | 5′GAAACCGTCGATATTCAGCCATGTGC |
| SEQ ID NO.: 59 | CTTCTTCCGCGTGCAGCAGATGGCGATG |
| | GCTGGTTTCCATCAGTTGCTG3′ |
| Oligo 59, | 5′CATGGCTGAATATCGACGGTTTCCAT |
| SEQ ID NO.: 60 | ATGGGGATTGGTGGCGACGACTCCTGGA |
| | GCCCGTCAGTATCGGCG3′ |
| Oligo 60, | 5′TTATTTTTGACACCAGACCAACTGGT |
| SEQ ID NO.: 61 | AATGGTAGCGACCGGCGCTCAGCTGGAA |
| | TTCCGCCGATACTGACGGGC3′ |
| LacZ gene- | 5′ATGACCATGATTACGGATTCACTGGC |
| SEQ ID NO: 62 | CGTCGTTTTACAACGTCGTGACTGGGAA |
| | AACCCTGGCGTTACCCAACTTAATCGCC |
| | TTGCAGCACATCCCCCTTTCGCCAGCTG |
| | GCGTAATAGCGAAGAGGCCCGCACCGAT |
| | CGCCCTTCCCAACAGTTGCGCAGCCTGA |
| | ATGGCGAATGGCGCTTTGCCTGGTTTCC |
| | GGCACCAGAAGCGGTGCCGGAAAGCTGG |
| | CTGGAGTGCGATCTTCCTGAGGCCGATA |
| | CTGTCGTCGTCCCCTCAAACTGGCAGAT |
| | GCACGGTTACGATGCGCCCATCTACACC |
| | AACGTGACCTATCCCATTACGGTCAATC |
| | CGCCGTTTGTTCCCACGGAGAATCCGAC |
| | GGGTTGTTACTCGCTCACATTTAATGTT |
| | GATGAAAGCTGGCTACAGGAAGGCCAGA |
| | CGCGAATTATTTTTGATGGCGTTAACTC |
| | GGCGTTTCATCTGTGGTGCAACGGGCGC |
| | TGGGTCGGTTACGGCCAGGACAGTCGTT |
| | TGCCGTCTGAATTTGACCTGAGCGCATT |
| | TTTACGCGCCGGAGAAAACCGCCTCGCG |
| | GTGATGGTGCTGCGCTGGAGTGACGGCA |
| | GTTATCTGGAAGATCAGGATATGTGGCG |
| | GATGAGCGGCATTTTCCGTGACGTCTCG |
| | TTGCTGCATAAACCGACTACACAAATCA |
| | GCGATTTCCATGTTGCCACTCGCTTTAA |
| | TGATGATTTCAGCCGCGCTGTACTGGAG |
| | GCTGAAGTTCAGATGTGCGGCGAGTTGC |
| | GTGACTACCTACGGGTAACAGTTTCTTT |
| | ATGGCAGGGTGAAACGCAGGTCGCCAGC |
| | GGCACCGCGCCTTTCGGCGGTGAAATTA |
| | TCGATGAGCGTGGTGGTTATGCCGATCG |
| | CGTCACACTACGTCTGAACGTCGAAAAC |
| | CCGAAACTGTGGAGCGCCGAAATCCCGA |
| | ATCTCTATCGTGCGGTGGTTGAACTGCA |
| | CACCGCCGACGGCACGCTGATTGAAGCA |
| | GAAGCCTGCGATGTCGGTTTCCGCGAGG |
| | TGCGGATTGAAAATGGTCTGCTGCTGCT |
| | GAACGGCAAGCCGTTGCTGATTCGAGGC |
| | GTTAACCGTCACGAGCATCATCCTCTGC |
| | ATGGTCAGGTCATGGATGAGCAGACGAT |
| | GGTGCAGGATATCCTGCTGATGAAGCAG |
| | AACAACTTTAACGCCGTGCGCTGTTCGC |
| | ATTATCCGAACCATCCGCTGTGGTACAC |
| | GCTGTGCGACCGCTACGGCCTGTATGTG |
| | GTGGATGAAGCCAATATTGAAACCCACG |
| | GCATGGTGCCAATGAATCGTCTGACCGA |
| | TGATCCGCGCTGGCTACCGGCGATGAGC |
| | GAACGCGTAACGCGAATGGTGCAGCGCG |
| | ATCGTAATCACCCGAGTGTGATCATCTG |
| | GTCGCTGGGGAATGAATCAGGCCACGGC |
| | GCTAATCACGACGCGCTGTATCGCTGGA |
| | TCAAATCTGTCGATCCTTCCCGCCCGGT |
| | GCAGTATGAAGGCGGCGGAGCCGACACC |
| | ACGGCCACCGATATTATTTGCCCGATGT |
| | ACGCGCGCGTGGATGAAGACCAGCCCTT |
| | CCCGGCTGTGCCGAAATGGTCCATCAAA |
| | AAATGGCTTTCGCTACCTGGAGAGACGC |
| | GCCCGCTGATCCTTTGCGAATACGCCCA |
| | CGCGATGGGTAACAGTCTTGGCGGTTTC |
| | GCTAAATACTGGCAGGCGTTTCGTCAGT |
| | ATCCCCGTTTACAGGGCGGCTTCGTCTG |
| | GGACTGGGTGGATCAGTCGCTGATTAAA |
| | TATGATGAAAACGGCAACCCGTGGTCGG |
| | CTTACGGCGGTGATTTTGGCGATACGCC |
| | GAACGATCGCCAGTTCTGTATGAACGGT |
| | CTGGTCTTTGCCGACCGCACGCCGCATC |
| | CAGCGCTGACGGAAGCAAAACACCAGCA |
| | GCAGTTTTTCCAGTTCCGTTTATCCGGG |
| | CAAACCATCGAAGTGACCAGCGAATACC |
| | TGTTCCGTCATAGCGATAACGAGCTCCT |
| | GCACTGGATGGTGGCGCTGGATGGTAAG |
| | CCGCTGGCAAGCGGTGAAGTGCCTCTGG |
| | ATGTCGCTCCACAAGGTAAACAGTTGAT |
| | TGAACTGCCTGAACTACCGCAGCCGGAG |
| | AGCGCCGGGCAACTCTGGCTCACAGTAC |
| | GCGTAGTGCAACCGAACGCGACCGCATG |
| | GTCAGAAGCCGGGCACATCAGCGCCTGG |
| | CAGCAGTGGCGTCTGGCGGAAAACCTCA |
| | GTGTGACGCTCCCCGCCGCGTCCCACGC |
| | CATCCCGCATCTGACCACCAGCGAAATG |
| | GATTTTTGCATCGAGCTGGGTAATAAGC |
| | GTTGGCAATTTAACCGCCAGTCAGGCTT |
| | TCTTTCACAGATGTGGATTGGCGATAAA |
| | AAACAACTGCTGACGCCGCTGCGCGATC |
| | AGTTCACCCGTGCACCGCTGGATAACGA |
| | CATTGGCGTAAGTGAAGCGACCCGCATT |
| | GACCCTAACGCCTGGGTCGAACGCTGGA |
| | AGGCGGCGGGCCATTACCAGGCCGAAGC |
| | AGCGTTGTTGCAGTGCACGGCAGATACA |
| | CTTGCTGATGCGGTGCTGATTACGACCG |
| | CTCACGCGTGGCAGCATCAGGGGAAAAC |
| | CTTATTTATCAGCCGGAAAACCTACCGG |
| | ATTGATGGTAGTGGTCAAATGGCGATTA |
| | CCGTTGATGTTGAAGTGGCGAGCGATAC |
| | ACCGCATCCGGCGCGGATTGGCCTGAAC |
| | TGCCAGCTGGCGCAGGTAGCAGAGCGGG |
| | TAAACTGGCTCGGATTAGGGCCGCAAGA |
| | AAACTATCCCGACCGCCTTACTGCCGCC |
| | TGTTTTGACCGCTGGGATCTGCCATTGT |
| | CAGACATGTATACCCCGTACGTCTTCCC |
| | GAGCGAAAACGGTCTGCGCTGCGGGACG |
| | CGCGAATTGAATTATGGCCCACACCAGT |
| | GGCGCGGCGACTTCCAGTTCAACATCAG |
| | CCGCTACAGTCAACAGCAACTGATGGAA |
| | ACCAGCCATCGCCATCTGCTGCACGCGG |
| | AAGAAGGCACATGGCTGAATATCGACGG |
| | TTTCCATATGGGGATTGGTGGCGACGAC |
| | TCCTGGAGCCCGTCAGTATCGGCGGAAT |
| | TCCAGCTGAGCGCCGGTCGCTACCATTA |
| | CCAGTTGGTCTGGTGTCAAAAATAA3′ |

TABLE 5
Table 5. PCA reaction mixture components for assembly
of the LacZ gene (SEQ ID NO.: 62) within nanoreactors.

| PCA reaction mixture | 1 (x100 ul) | Final conc. | |
|---|---|---|---|
| H₂O | 62.00 | | |
| 5x Q5 buffer | 20.00 | 1x | |
| 10 mM dNTP | 1.00 | 100 | uM |
| BSA 20 mg/ml | 5.00 | 1 | mg/ml |
| Oligonucleic acid mix (50 nM each) | 10.00 | 5 | nM |

| | | |
|---|---|---|
| Q5 polymerase 2 U/ul | 2.00 | 2 U/50 ul |

PCA reaction mixture drops of about 400 nL were dispensed using a Mantis dispenser (Formulatrix, MA) on the top of channels of a device side of a three-dimensional substrate having a plurality of loci channels in fluid communication with a single well of a cluster. A nanoreactor chip was manually mated with the substrate to pick up the droplets having the PCA reaction mixture and oligonucleic acids from each channel. The droplets were picked up into individual nanoreactors in the nanoreactor chip by releasing the nanoreactor from the substrate immediately after pick up. The nanoreactors were sealed with a heat sealing film, placed in a thermocycler for PCA. PCA thermocycling conditions are shown in Table 6. An aliquot of 0.5 ul was collected from 1-10 individual wells and the aliquots were amplified in plastic PCR tubes using forward primer (5′ATGACCATGATTACGGATTCACTGGCC3′ (SEQ ID NO.:63)) and reverse primer (5′TTATTTTTGACACCAGACCAACTGGTAATGG3′ (SEQ ID NO.:64)). Thermocycling conditions for PCR are shown in Table 7 and PCR reaction components are shown in Table 8. The amplification products were ran on a BioAnalyzer DNA 7500 instrument and on a DNA agarose gel. The gel showed products 1-10 having a size slightly larger than 3000 bp (not shown). A PCA reaction performed in plastic tube was also run as a positive control. A PCR reaction ran without a PCA template served as a negative control.

TABLE 6
Table 6. PCA thermocycling conditions for
assembly of the LacZ gene (SEQ ID NO.: 62).

| No. of cycles | Temperature (° C.) | Time |
|---|---|---|
| 1 | 98 | 45 seconds |
| 40 | 98 | 15 seconds |
| | 63 | 45 seconds |
| | 72 | 60 seconds |
| 1 | 72 | 5 minutes |
| 1 | 4 | Hold |

TABLE 7
Table 7. PCR thermocycling conditions for the amplification
of the LacZ gene (SEQ ID NO.: 62) assembled by PCA.

| No. of cycles | Temperature (° C.) | Time |
|---|---|---|
| 1 | 98 | 30 seconds |
| 30 | 98 | 7 seconds |
| | 63 | 30 seconds |
| | 72 | 90 seconds |
| 1 | 72 | 5 minutes |
| 1 | 4 | Hold |

TABLE 8
Table 8. PCR reaction mixture components for the amplification
of the LacZ gene (SEQ ID NO.: 62) assembled by PCA.

| | | Final |
|---|---|---|
| PCR reaction mixture | Volume (ul) | concentration |
| H2O | 17.50 | |
| 5x Q5 buffer | 5.00 | 1x |
| 10 mM dNTP | 0.50 | 200 uM |
| F-primer 20 uM | 0.63 | 0.5 uM |
| R-primer 20 uM | 0.63 | 0.5 uM |
| BSA 20 mg/ml | 0.00 | |
| Q5 pol 2 U/ul | 0.25 | 1 U/50 ul |
| Template (PCA assembled | 0.50 | 1 ul/50 ul rxn |
| product) | | |

### Example 3

### Cell-Free Sorting and Cloning of Heterogeneous Sequence Populations

A sample of double-stranded target nucleic acids with heterogeneous sequence populations was partitioned using cell-free cloning to separate the target nucleic acids by sequence. The sample comprised a synthesized gene fragment construct comprising a population of nucleic acids having a predetermined sequence and one or more nucleic acids having sequences that differed from the predetermined nucleic acid sequence by one or more bases. The construct was purchased as a single gBlock from IDT. The predetermined sequence is indicated by SEQ ID NO.: 65:

5′CAGCAGTTCCTCGCTCTTCTCACGACGAGTTCGACATCAACAAGC
TGCGCTACCACAAGATCGTGCTGATGGCCGACGCCGATGTTGACGGC
CAGCACATCGCAACGCTGCTGCTCACCCTGCTTTTCCGCTTCATGCC
AGACCTCGTCGCCGAAGGCCACGTCTACTTGGCACAGCCACCTTTGT
ACAAACTGAAGTGGCAGCGCGGAGAGCCAGGATTCGCATACTCCGAT
GAGGAGCGCGATGAGCAGCTCAACGAAGGCCTTGCCGCTGGACGCAA
GATCAACAAGGACGACGGCATCCAGCGCTACAAGGGTCTCGGCGAGA
TGAACGCCAGCGAGCTGTGGGAAACCACCATGGACCCAACTGTTCGT
ATTCTGCGCCGCGTGGACATCACCGATGCTCAGCGTGCTGATGAACT
GTTCTCCATCTTGATGGGTGACGACGTTGTGGCTCGCCGCAGCTTCA
TCACCCGAAATGCCAAGGATGTTCGTTTCCTCGATATCTAAAGCGCC
TTACTTAACCCGCCCCTGGAATTCTGGGGGCGGGTTTTGTGATTTTT
AGGGTCAGCACTTTATAAATGCAGGCTTCTATGGCTTCAAGTTGGCC
AATACGTGGGGTTGATTTTTTAAAACCAGACTGGCGTGCCCAAGAGC
TGAACTTTCGCTAGTCATGGGCATTCCTGGCCGGTTTCTTGGCCTTC
AAACCGGACAGGAATGCCCAAGTTAACGGAAAAACCGAAAGAGGGGC
ACGCCAGTCTGGTTCTCCCAAACTCAGGACAAATCCTGCCTCGGCGC
CTGCGAAAAGTGCCCTCTCCTAAATCGTTTCTAAGGGCTCGTCAGAC
CCCAGTTGATACAAACATACATTCTGAAAATTCAGTCGCTTAAATGG
GCGCAGCGGGAAATGCTGAAAACTACATTAATCACCGATACCCTAGG
GCACGTGACCTCTACTGAACCCACCACCACAGCCCATGTTCCACTAC
CTGATGGATCTTCCACTCCAGTCCAAATTTGGGCGTACACTGCGAGT
CCACTACGAT3′.

Prior to sorting, the double-stranded nucleic acids of the sample were circularized by ligating sticky ends of the gene fragment nucleic acids to sticky ends of an adapter.

Generation of Gene Fragments with Sticky Ends Using Uracil Containing Primers

To generate sticky ends, uracil bases were added near the 5′ ends of each strand of the double-stranded gene fragment and the fragment was treated with a mixture of Uracil DNA glycosylase (UDG) and Endonuclease VIII (EndoVIII). The uracil bases were added to the gene fragment by amplifying the gene fragment with uracil containing primers (forward primer (5′CAGCAGT/ideoxyU/CCTCGCTCTTCT3′; SEQ ID NO.: 66) and reverse primer (5′ATCGTAG/ideoxyU/GGACTCGCAGTGTA3′; SEQ ID NO.: 67) by polymerase chain reaction (PCR). The PCR reaction was performed on a 50 uL PCR reaction mixture having components shown in Table 9 using the reaction conditions of Table 10.

TABLE 9
Table 9. PCR reaction mixture components for incorporating
uracil containing primers into a gene fragment.

| | | | Final | |
|---|---|---|---|---|
| PCR reaction mixture | Volume (ul) | | concentration | |
| 5x HF buffer (ThermoFisher Scientific) | 10 | | 1x | |
| 10 mM dNTP (NEB) | 0.8 | | 160 | uM |
| Primer SEQ ID NO.: 66 | 2.5 | ul | 10 | uM |
| Primer SEQ ID NO.: 67 | 2.5 | ul | 10 | uM |
| Phusion-U hot start DNA polymerase | 0.5 | ul | | |
| (ThermoFisher Scientific) | | | | |
| Gene fragment template | 1 | ng | 1 | ng |

| | |
|---|---|
| Water | up to 50 ul |

TABLE 10
Table 10. PCR reaction conditions for incorporating
uracil containing primers into a gene fragment.

| No. of cycles | Temperature (° C.) | Time |
|---|---|---|
| 1 | 98 | 30 seconds |
| 20 | 98 | 10 seconds |
| | 68 | 15 seconds |
| | 72 | 60 seconds |
| 1 | 72 | 5 minutes |
| 1 | 4 | Hold |

The PCR products comprising the gene fragments having 5′ uracils were purified using Qiagen MinElute column, eluted in 10 uL EB buffer, and analyzed by gel electrophoresis using a Bioanalyzer DNA7500 instrument (Agilent). The electrophoresis trace is provided in FIG. 7, which shows the amplified product with a peak around 1040 base pairs. The concentration of the purified gene fragment was 93 ng/ul, as measured using a NanoDrop instrument. The uracil-containing gene fragments were then digested at 37° C. for 30 minutes in a digestion reaction (15 nM of uracil containing gene fragments, 10 uL of 10× CutSmart buffer (NEB), 2 uL of UDG/EndoVIII (NEB or Enzymatics) and water up to 94.7 uL) to generate gene fragments having sticky ends.

Preparation of Circularized Gene Fragments

A double-stranded adapter sequence having 3′ overhangs (sticky ends) was ligated to the gene fragments having sticky ends. The first strand of the adapter had a 5′ phosphate for ligation. The second strand of the adapter lacked a base on its 5′ end so that a nucleotide gap was created after the adapter was ligated with the gene fragment. The second strand also did not have a 5′ phosphate to prevent ligation with the gene fragment at the 5′ lacking end. In order to prevent exonuclease digestion of the second strand, the first 6 phosphate bonds were phosphorothioated. The first strand of the adapter sequence is indicated by SEQ ID NO.: 68 (5′/5phos/TACGCTCTTCCTCAGCAGTGGTCATCGTAGT3′). The second strand of the adapter sequence is indicated by SEQ ID NO.: 69 (5′A*C*C*A*C*T*GCTGAGGAAGAGCGTACAGCAGTT3′), wherein * denotes a phosphorothioated bond. The first and second strands of the adapter were annealed by combining 5 uM of each strand in 1× CutSmart buffer (NEB), incubating at 95° C. for 5 min, followed by a slow cool.

The gene fragments having sticky ends were circularized by ligation to the adapter nucleic acid. Ligation occurred by mixing 94.7 uL of the gene fragments having sticky ends with 0.3 uL of the adapter (5 uM), 5 uL of 10 mM ATP, and 1 uL T4 DNA ligase (400 U/uL, NEB); followed by incubation at 21° C. for 15 min, 14° C. for 15 min, and then 4° C. for 10 min. The ligated, circularized dsDNA gene fragments comprised a) a continuous circularized strand comprising the first adapter strand ligated to a first strand of the gene fragment, and b) a discontinuous nicked strand comprising the second adapter strand and a second strand of the gene fragment; wherein the nicked strand comprised a gap between the 5′ strand of the second adapter strand and the second strand of the gene fragment; and wherein the continuous strand and the discontinuous strand were hybridized.

DNA that was not circularized by the ligation reaction was digested by exonuclease treatment. The phosphorothioated bonds of the nicked strand served to prevent digestion of the nicked strand by the exonuclease. Exonuclease treatment occurred by supplementing the ligation reaction products with 0.5 uL Exonuclease I (NEB, 20 U/uL) and 1.5 uL T7 Exonuclease (NEB, 10 U/uL), and incubating at 25° C. for 45 min, 37° C. for 15 min, then 80° C. for 20 min (for exonuclease deactivation). Exonuclease treated, circularized gene fragments were purified using Qiagen MinElute and ERC kit and eluted in 10 uL EB buffer. The circularized gene fragments were eluted at a concentration of 9.5 ng/uL (14.4 nM), as quantified using Qubit BR dsDNA kit (Life Technologies), and subsequently diluted to a concentration of 1 pM.

RCA of Circularized Gene Fragments

The purified nicked, circularized dsDNA gene fragments were diluted to a final concentration of 100 fM in a RCA reaction mixture (3 uL of 1 pM dsDNA; 3 uL 10×phi29 buffer; 0.75 uL dNTP; 0.60 uL BSA; 0.90 uL phi29 (10 U/uL; Enzymatics); 21.75 ul water). RCA was performed by incubating the reaction mixture at 30° C. for 1 hr, followed by 70° C. for 10 min. The discontinuous nicked strand of the circularized dsDNA served as the primer and the continuous strand of the circularized dsDNA served as the template DNA for the RCA reaction. Similar RCA reactions were successfully performed on RCA reaction mixtures having between 1 fM and 100 pM of circularized dsDNA.

Amplification of Single Molecule RCA Products

RCA amplification products were diluted by 10⁴-fold in a 0.1% polysorbate-20 (polyoxyethylene (20) sorbitan monolaurate) solution so that on average there were about 1.2 molecules per 0.2 uL of solution. A 0.2 uL aliquot having, on average, 1.2 molecules of RCA product (a clonal fraction having on average, a single parent molecule), was used as a template for a PCR reaction. In other experiments, a multiple displacement amplification (MDA) reaction was performed either prior to, or as an alternative to, PCR. PCR reaction mixture conditions are shown in Table 11. PCR was performed on single molecule fractions using the thermocycling steps of Table 12. On average, 12 to 24 of the single molecule PCR reactions were performed using the methods of this example.

TABLE 11
PCR reaction mixture components for
amplification of partitioned single
molecule RCA products.

| | Volume |
|---|---|
| PCR reaction mixture | (ul) |
| NEB Q5 mastermix (NEB) | 10 uL |
| Forward primer (10 uM); SEQ ID NO.: 70 | 1 uL |
| (5′CAG CAG TTC CTC GCT CTT CT3′) | |
| Reverse primer (10 uM); SEQ ID NO.: 71 | 1 uL |
| (5′ATC GTA GTG GAC TCG CAG TGT A3′) | |
| Water | 7.8 uL |
| Diluted RCA product | 0.2 uL |

TABLE 12
Table 12. PCR reaction conditions for amplification
of partitioned single molecule RCA products.

| No. of cycles | Temperature (° C.) | Time |
|---|---|---|
| 1 | 98 | 30 seconds |
| 40 | 98 | 10 seconds |
| | 69 | 15 seconds |
| | 72 | 30 seconds |
| 1 | 72 | 5 minutes |
| 1 | 4 | 1 minute |

PCR amplification products were analyzed using a Bioanalyzer DNA 7500 instrument (Agilent) or a Fragment Analyzer™ (Advanced Analytical).

Sequence Analysis of Amplified Clonal Fractions

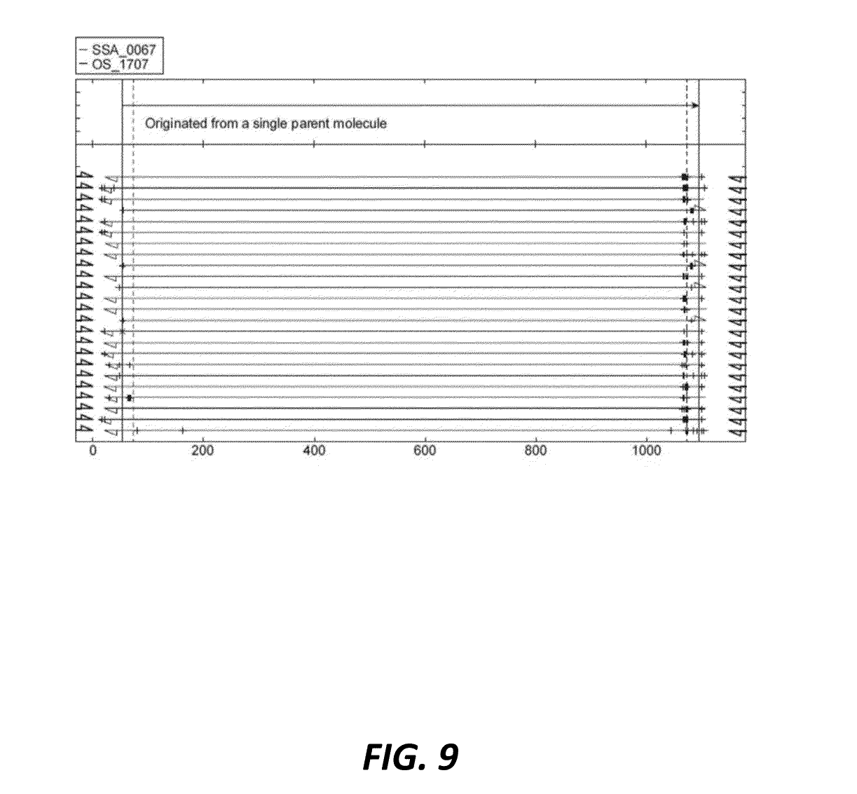

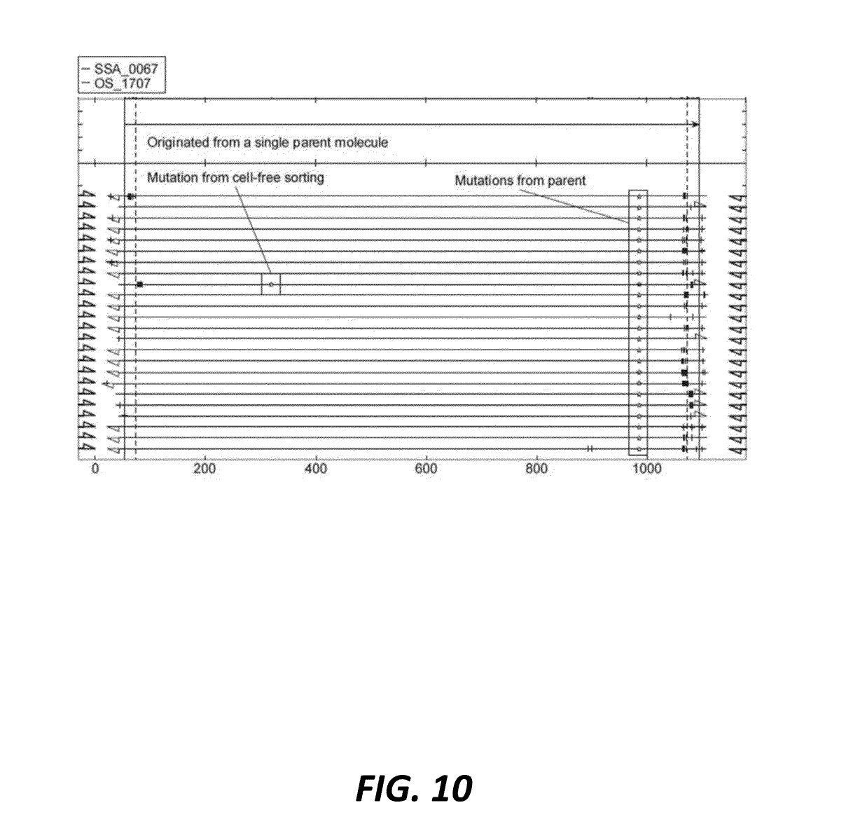

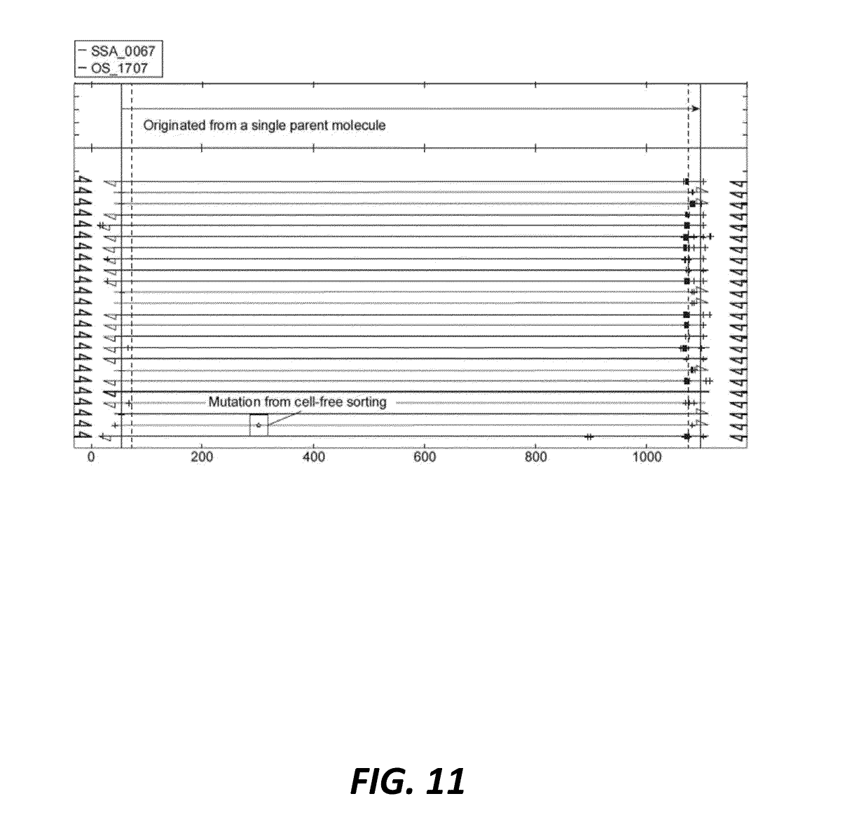

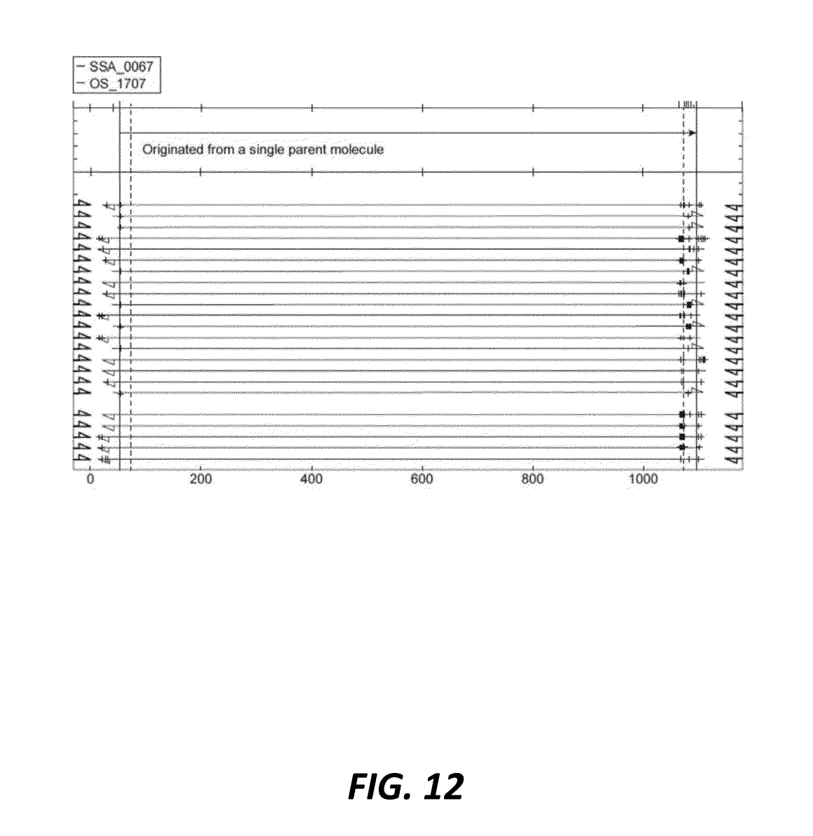

The resulting amplification products were sequenced by Sanger sequencing. The sequence alignment maps for clonal samples numbers 1-5 are shown in FIGS. 8-12, respectively. As shown in FIG. 8, all sequences within clonal sample number 1 had the same mutation as the parent molecule (the fractionated, single molecule), as indicated by an asterisk. In addition, one of the amplified nucleic acids had an additional random mutation. As shown in FIG. 9, all sequences within clonal sample number 2 had no mutations, i.e. all sequences had the predetermined sequence (SEQ ID NO.: 63) of their parent molecule. As shown in FIG. 10, all sequences within clonal sample number 3 had the same mutation as the parent molecule (the fractionated, single molecule), as indicated by an asterisk. In addition, one of the amplified nucleic acids had an additional random mutation. As shown in FIG. 11, all sequences within clonal sample number 4 had the predetermined sequence (SEQ ID NO.: 63) of their parent molecule, with the exception of one sequence having a random mutation. As shown in FIG. 12, all sequences within clonal sample number 5 had no mutations, i.e. all sequences had the predetermined sequence (SEQ ID NO.: 63) of their parent molecule.

An RCA amplification product obtained prior to clonal fractionation was also sequenced by Sanger sequencing. This RCA amplification product was diluted 100× to contain amplicons of about 100 parent nucleic acids. The sequence alignment map is provided in FIG. 13. FIG. 13 shows that a plurality of parent sequences were present prior to single molecule fractionation (clonal sorting). In contrast, the clonally sorted samples (as represented in FIGS. 8-12) contained clonally amplified fractions that were highly similar, if not identical. The small variations in sequences within a fraction were likely introduced during PCR amplification and are in the vicinity of polymerase error rate.

### Example 4

### Clonal Sorting of a Two-Component Sample

A sample of double-stranded target nucleic acids having two populations of sequence distinct nucleic acids was partitioned using cell-free cloning. This sample was sequenced prior to sorting to illustrate the two distinct sequence populations. The sequencing traces are shown in FIG. 14. One population of nucleic acids had a predetermined sequence without any errors. Another population of nucleic acids had the predetermined sequence with two different mutations, indicated by the cross and asterisk in FIG. 14.

The sample was diluted to a concentration that was calculated to provide, on average, 1.2 molecules per fraction after sorting. The sample was then partitioned into 24 fractions and amplified by PCR. The amplification products from each fraction were visualized by gel electrophoresis and are shown in FIGS. 15A-15B. As shown in FIGS. 15A-15B, 17 of the 24 fractions (71%) comprised amplifiable nucleic acid material. It was estimated that 72% of the fractions would contain amplifiable nucleic acid material using a Poisson distribution.

The sample was similarly diluted to a concentration that was calculated to provide, on average, 0.6 molecules per fraction after sorting. The sample was then partitioned into 24 fractions and amplified by PCR. The amplification products from each fraction were visualized by gel electrophoresis and are shown in FIGS. 15C-15D. As shown in FIGS. 15C-15D, 13 of the 24 fractions (54%) comprised amplifiable nucleic acid material. It was estimated that 47% of the fractions would contain amplifiable nucleic acid material using a Poisson distribution. Fractions 9 and 10 were sequenced by Sanger sequencing and their traces are shown in FIGS. 16 and 17, respectively. As shown in FIG. 16, fraction 9 had nucleic acids with the predetermined sequence without any errors. As shown in FIG. 17, fraction 10 had nucleic acids with the predetermined sequence with errors.

### Example 5

### Clonal Sorting of a Two-Component Sample Using Single Molecule RCA

A sample of double-stranded target nucleic acids having two populations of sequence distinct nucleic acids was partitioned into single molecule fractions, followed by amplification by RCA. The sample comprised a first plasmid having a 322 base pair insert and a second plasmid having a 724 base pair insert. The mixed population sample was prepared by combining a 1 ul (2 ng) aliquot of the first plasmid and a 1 ul (2 ng) aliquot of the second plasmid with 998 ul of TE buffer (supplemented with 0.2% Tween 20) in a low binding 1.5 ml tube. To prepare single molecule samples from the mixed population sample, serial dilutions were performed to generate dilutions having, on average, 97 (dilution A), 9.7 (dilution B), or 0.97 (dilution C) molecules per 0.6 ul fraction.

Single Molecule RCA

Fractions partitioned from dilutions A-C were amplified using RCA. The RCA reaction mixtures were prepared by two methods. In the first method, the following were first combined in a reaction mixture: 1×phi29 buffer, 1 mM each dNTPs, 1 mM DTT, 0.02% Tween 20, 1×BSA, and 1 U/ml yeast pyrophosphatase. Phi29 DNA polymerase was added, and the reaction mixture was incubated at room temperature for 10 min. Following incubation, a pre-heated, diluted sample (dilution A, B or C pre-heated to 95° C. for 3 min, followed by cooling on ice for 5 min) and primers were added to the reaction mixture.

In the second method, the following were first combined in a reaction mixture: 1×phi29 buffer, 1 mM each dNTPs, 1 mM DTT, 0.02% Tween 20, primers, and a diluted sample (dilution A, B or C). The mixture was heated to 95° C. for 3 min and then cooled on ice for 5 min. The cooled mixture was then combined with a pre-mixed combination of phi29 DNA polymerase, yeast pyrophosphatase and BSA.

For both methods, the final RCA reaction volumes were 0.6 ul. Each 0.6 ul reaction was overlaid with 100 ul of mineral oil and then incubated at 30° C. for 6 hr for amplification by RCA. Eight RCA reactions were performed for each dilution A, B and C, using either the first or the second reaction mixture preparation methods. In addition, 8 RCA reactions were performed that did not contain template DNA (control), using either the first or the second reaction mixture preparation methods.

Amplification of RCA Products

RCA reaction products were supplemented with 25 ul of a PCR reaction mix (having Thermo Phusion DNA polymerase and a standard plasmid M13 primer pair) for PCR. The amplified PCR products were visualized by gel electrophoresis and are shown in FIG. 18A-18B. FIG. 18A shows the PCR products that were amplified from RCA products amplified using the first method of RCA reaction preparation. As shown in FIG. 18A, no PCR products having the expected insert size of 890 (724+M13 primers) or 488 (322+M13 primers) base pairs were observed. In contrast, FIG. 18B shows PCR products that were amplified from RCA products amplified using the second method of RCA reaction preparation. For the RCA reactions that had, on average, 97 molecules per fraction, 3 out of 8 fractions contained the first plasmid (322 base pair insert, 488 base pairs after amplification with M13 primers) 2 out of 8 fractions had the second plasmid (724 base pair insert, 890 base pairs after amplification with M13 primers), and 1 out of the 8 fractions was monoclonal. For the RCA reactions that had, on average, 9.7 molecules per fraction, 0 out of 8 fractions contained the first plasmid (322 base pair insert, 488 base pairs after amplification with M13 primers) 1 out of 8 fractions was monoclonal and only contained the second plasmid (724 base pair insert, 890 base pairs after amplification with M13 primers). For the RCA reactions that had, on average, 0.97 molecules per fraction, 4 out of 8 fractions contained the first plasmid (322 base pair insert, 488 base pairs after amplification with M13 primers), 3 out of 8 fractions had the second plasmid (724 base pair insert, 890 base pairs after amplification with M13 primers); where 4 out of the 8 fractions contained monoclonal nucleic acid populations.

### Example 6

### Clonal Sorting of a Two-Component Sample Using Single Molecule RCA in Nanowells

A sample of double-stranded target nucleic acids having two populations of sequence distinct nucleic acids was partitioned into single molecule fractions in nanowells, followed by amplification by RCA. The sample comprised a first plasmid having a 844 base pair insert and a second plasmid having the same 844 base pair insert but with a C to T mutation at base 794. The mixed population sample was prepared by combining the first plasmid and second plasmid with water and 0.2% Tween 20 in a low binding 1.5 ml tube. To prepare single molecule samples from the mixed population sample, serial dilutions were performed to generate dilutions having, on average, 4.7 (dilution A) or 0.47 (dilution B) molecules per 0.3 ul fraction.

Single Molecule RCA

Fractions partitioned from dilutions A or B were amplified using RCA. In addition, control samples not having template were also subject to RCA reaction conditions. Each dilution or control sample was partitioned and amplified by RCA in separate fractions. The RCA reaction mixtures were prepared by first mixing 3.54 ul water, 2 ul of 10×phi29 buffer, 3 ul of 10 mM dNTPs, 0.6 ul of 100 mM DTT, 0.6 ul of 10% Tween 20, 3 ul of 0.5 mM random hexamer primers, and 6.26 ul template (water for control, dilution A or dilution B); and incubating this first mixture at 95° C. for 3 min, followed by cooling on ice for 5 min. A second mixture was prepared by mixing 6.18 ul water, 1 ul of 10×phi29 polymerase buffer, 0.6 ul of 100 mg/ml BSA, 0.6 ul of 0.1 U/ul IPP and 1.62 ul of 10 U/ul phi29 DNA polymerase. Aliquots (0.2 ul) of the first mixture were dispensed into nanowells, followed by aliquots (0.1 ul) of the second enzyme mixture. 16 nanowells contained control samples without template DNA, 17 nanowells contained, on average, 4.7 molecules of template (dilution A), and 16 nanowells contained, on average, 0.47 molecules of template (dilution B). Each 0.3 ul reaction was overlaid with mineral oil to prevent evaporation. RCA was performed by incubating the wells at 30° C. for 18 hours. The phi29 DNA polymerase was then inactivated at 72° C. for 10 min.

Using similar reaction conditions as described for the RCA reaction described above, RCA was performed using control, dilution A, or dilution B samples in 0.6 ul reaction volumes in plastic tubes. As the volume was doubled, tubes with dilution A had, on average, 9.4 molecules per tube and tubes with dilution B had, on average, 0.94 molecules per tube. RCA was performed with 8 tubes each of control, dilution A and dilution B.

Amplification of RCA Products

RCA reactions were recovered from each nanowell or tube and supplemented with 25 ul of a PCR reaction mix (having Thermo Phusion DNA polymerase and a standard plasmid M13 primer pair) for PCR. Each RCA product was subject to amplification by PCR using the reaction conditions in Table 13.

TABLE 13
Table 13. PCR reaction conditions
for amplification of RCA products.

| No. of cycles | Temperature (° C.) | Time |
|---|---|---|
| 1 | 98 | 30 seconds |
| 40 | 98 | 10 seconds |
| | 71 | 45 seconds |
| | 72 | 45 seconds |
| 1 | 72 | 5 minutes |

The amplified PCR products were visualized by gel electrophoresis and are shown in FIGS. 19A-19). FIG. 19A shows the PCR products that were amplified from RCA products amplified in nanowells. For the RCA reactions that had, on average, 4.7 molecules per fraction, 12 out of 17 fractions contained an amplification product (around 850 bp). For RCA reactions that had, on average, 0.47 molecules per fraction, 6 out of 16 fractions contained an amplification product (around 850 bp).

FIG. 19B shows the PCR products that were amplified from RCA products amplified in tubes. For the RCA reactions that had, on average, 9.4 molecules per fraction, 8 out of 8 fractions contained an amplification product (around 850 bp). For RCA reactions that had, on average, 0.94 molecules per fraction, 5 out of 8 fractions contained an amplification product (around 850 bp).

Sequence Analysis of Amplified Clonal Fractions

A selection of PCR amplification products from the clonal fractions were sequenced by Sanger sequencing. A list of the PCR amplification products sequenced is shown in Table 14. The details of the sequencing results for the sequenced PCR products are shown in Table 15.

TABLE 14
Table 14. Fraction details for PCR
amplification products sequenced.

| PCR | RCA in | Average molecule/ | Fraction |
|---|---|---|---|
| product name | nanowell or tube | fraction | No. |
| NW-E7-12 | Nanowell | 4.7 | 12 |
| NW-E7-15 | Nanowell | 4.7 | 15 |
| NW-E8-5 | Nanowell | 0.47 | 5 |
| NW-E8-9 | Nanowell | 0.47 | 9 |
| NW-E8-10 | Nanowell | 0.47 | 10 |
| NW-E8-14 | Nanowell | 0.47 | 14 |
| NW-E8-15 | Nanowell | 0.47 | 15 |
| NW-E8-16 | Nanowell | 0.47 | 16 |
| TB-E8-2 | Tube | 0.94 | 2 |
| TB-E8-3 | Tube | 0.94 | 3 |
| TB-E8-7 | Tube | 0.94 | 7 |
| TB-E8-8 | Tube | 0.94 | 8 |

TABLE 15
Table 15. Sequence identities of PCR amplification products
sequenced from each fraction described in Table 14.

| PCR product name | Sequence identity | Clonality |
|---|---|---|
| NW-E7-12 | C794T mutation | monoclonal |
| NW-E7-15 | no mutation at 794 | monoclonal |
| NW-E8-5 | C794T mutation | monoclonal |
| NW-E8-9 | no mutation at 794 | monoclonal |
| NW-E8-10 | no mutation at 794 | monoclonal |
| NW-E8-14 | no mutation at 794 | monoclonal |
| NW-E8-15 | C794T mutation | monoclonal |
| NW-E8-16 | C794T mutation | monoclonal |
| TB-E8-2 | no mutation at 794 | monoclonal |
| TB-E8-3 | no mutation at 794 | monoclonal |
| TB-E8-7 | C794T mutation | monoclonal |
| TB-E8-8 | no mutation at 794 | monoclonal |

As shown in Table 15, all fractions had a monoclonal population of nucleic acids (i.e. each nucleic acid sequenced within the fraction had the same sequence as the other nucleic acids within the same fraction). This experiment demonstrates cell-free cloning methods disclosed herein performed in small volumes of nanowells. In addition, RCA was performed on single molecule fractions within a nanowell, and the resulting RCA products were removable from the nanowells, amplified by PCR and sequenced.

### Example 7

### Cell-Free Cloning of DNA Circularized with Hairpins

A clonal population of double-stranded template nucleic acids was circularized by ligation with hairpin DNA, followed by amplification of the circularized ligation products by RCA. The RCA amplification products were partitioned into single molecule fractions and amplified to generate fractions comprising monoclonal copies of the parent single molecules. The template nucleic acid comprised a first double-stranded nucleic acid having 844 base pairs and a second double-stranded nucleic acid having the same sequence as the first double-stranded nucleic acid, but with a C to T mutation at base 794.

Circularization of Template DNA by Ligation with DNA Hairpins

To prepare template dsDNA for ligation, uracil bases were added near the 5′ ends of each strand of the dsDNA templates by PCR, as described in Example 3. The uracil containing amplicons were digested with UDG and EndoVIII to generate dsDNA with 3′ overhangs.

Preparation of Circularized Template DNA

The prepared dsDNA templates comprising sticky ends were ligated to sticky ends of hairpin A at one end of the templates and sticky ends of hairpin B at the other end of the templates. The sequences for hairpins A and B with sticky ends are shown in Table 16. The loop region of each hairpin is underlined.

TABLE 16
Hairpin sequences ligated to target
nucleic acids to generate circularized
nucleic acids.

| Sequence Name | Sequence |
|---|---|
| Hairpin A; | /5Phos/CTCTCTCTTTTCCTCCTCCTC |
| SEQ ID NO.: 72 | CGTTGTTGTTGTTGAGAGAGTCGACTGT |
| Hairpin B; | /5Phos/GAGCTGCCCCACCATCCACCC |
| SEQ ID NO.: 73 | GTATCTCATCCAAGCAGCTCCTGTTGCT |

Ten different ligation reactions were performed using the reaction mixtures outline in Table 17. For samples C2 to C9, after addition of USER enzyme, the ligation reactions were incubated at 37° C. for 30 min. For sample C10, the ligation reaction was incubated at 37° C. for 30 min without the addition of USER enzyme. Following incubation at 37° C. for 30 min, samples C2 to C10 were supplemented with T4 DNA ligase and incubated at 25° C. for 15 minutes for ligation. Following ligation, each reaction was digested with 50 U of ExoIII (NEB), 10 U of ExoI (NEB) at 37° C. for 1 hour to digest non circularized DNA.

TABLE 17
Table 17. Reaction conditions for the ligation of hairpins to target nucleic acids to generate
circularized target nucleic acids.

| | Sample | | | | | | | | | |
|---|---|---|---|---|---|---|---|---|---|---|
| | C1 | C2 | C3 | C4 | C5 | C6 | C7 | C8 | C9 | C10 |
| Hairpin | 20x | 20x | 2x | 4x | 8x | 20x | 2x | 4x | 8x | 20x |
| DNA: | | | | | | | | | | |
| template | | | | | | | | | | |
| 1 mM ATP | 5 | 5 | 5 | 5 | 5 | 0.5 | 0.5 | 0.5 | 0.5 | 5 |
| (ul) | | | | | | | | | | |
| Water (ul) | 5.1 | 3.1 | 3.1 | 7.3 | 6.5 | 9.6 | 9.6 | 11.8 | 11 | 4.1 |
| 10x buffer, | 2 | 2 | 2 | 2 | 2 | 2 | 2 | 2 | 2 | 2 |
| no ATP (ul) | | | | | | | | | | |
| 0.26 uM | 3.9 | 3.9 | 3.9 | 3.9 | 3.9 | 3.9 | 3.9 | 3.9 | 3.9 | 3.9 |
| template (ul) | | | | | | | | | | |
| 2 uM or 20 | 2 | 2 | 2 | 0.4 | 0.8 | 2 | 2 | 0.4 | 0.8 | 2 |
| uM hairpin A | | (20 uM) | (2 uM) | (20 uM) | (20 uM) | (20 uM) | (2 uM) | (20 uM) | (20 uM) | |
| (ul) | | | | | | | | | | |
| 2 uM or 20 | 2 | 2 | 2 | 0.4 | 0.8 | 2 | 2 | 0.4 | 0.8 | 2 |
| uM hairpin B | | (20 uM) | (2 uM) | (20 uM) | (20 uM) | (20 uM) | (2 uM) | (20 uM) | (20 uM) | |
| (ul) | | | | | | | | | | |
| USER | 0 | 1 | 1 | 1 | 1 | 1 | 1 | 1 | 1 | 0 |
| enzyme | | | | | | | | | | |
| (1 U/ul) | | | | | | | | | | |
| T4 DNA | 0 | 1 | 1 | 1 | 1 | 1 | 1 | 1 | 1 | 1 |
| Ligase | | | | | | | | | | |
| (400 U/ul) | | | | | | | | | | |

DNA from each circularization reaction C1-C10 was separated by gel electrophoresis, and is shown in FIG. 20. Control lanes C1 and C10 show the 844 template and hairpin DNAs. Lanes corresponding to ligation reactions C2-C9 show a slightly higher band indicative of template DNA ligated to the hairpin DNAs.

DNA that was not circularized by the ligation reaction was digested by exonuclease treatment. The phosphorothioated bonds of the nicked strand served to prevent digestion of the nicked strand by the exonuclease. Exonuclease treatment occurred by supplementing the ligation reaction products with 0.5 uL Exonuclease I (NEB, 20 U/uL) and 1.5 uL T7 Exonuclease (NEB, 10 U/uL), and incubating at 25° C. for 45 min, 37° C. for 15 min, then 80° C. for 20 min (to deactivate the exonucleases). Exonuclease treated, circularized gene fragments were purified using Qiagen MinElute and ERC kit and eluted in 10 uL EB buffer. The circularized gene fragments were eluted at a concentration of 9.5 ng/uL (14.4 nM), as quantified using Qubit BR dsDNA kit (Life Technologies), and subsequently diluted to a concentration of 1 pM.

RCA of Circularized Bell DNA

Single-stranded circularized DNA (or bell DNA) was amplified by RCA. Briefly, 32 ul of water, 5 ul of 10×phi29 buffer, 2.5 ul of 10 mM dNTPs, 2.5 ul of 1 uM hairpin primer A or hairpin primer B, and 1.14 ul purified circularized DNA (about 5.4×10⁷ copies in final mixture) were combined in a first RCA reaction mixture, heated at 72° C. for 2 min, and cooled on ice for 5 min. The sequences for hairpin primers are shown in Table 18. A second RCA reaction mixture comprising 2 ul of phi29 DNA polymerase (NEB), 0.5 ul of 0.05 U inorganic pyrophosphatase, 1 ul of 10 mg/ml BSA (NEB), and 1 ul of 100 mM DTT, was added to the first RCA reaction mixture, and the combination was incubated at 30° C. for 1 hour for RCA. The final concentration of RCA amplification products (DNA nanoballs) was 1.08×10⁶ copies/ul.

TABLE 18
Hairpin primer sequences for amplification
of target nucleic acids circularized by
ligation with hairpins.

| Sequence Name | Sequence |
|---|---|
| Hairpin primer A; | G*G*AGGAGGAGGA |
| SEQ ID NO.: 74 | |
| Hairpin primer A; | G*A*TACGGGTGGA |
| SEQ ID NO.: 75 | |

Amplification of Single Molecule RCA Products

RCA amplification products (DNA nanoballs) were diluted in 0.1% Tween 20, TE buffer and used as templates in PCR reactions, which were performed essentially as described in previous examples. PCR reactions were performed on 12 fractions having, on average, 10.8 DNA nanoballs and 12 fractions having, on average, 1.08 DNA nanoballs. PCR amplification products were visualized by gel electrophoresis and the digital images are shown in FIG. 21A-21B. FIG. 21A shows that all 12 of the PCR fractions having, on average, 10.8 copies of DNA nanoballs as starting material were successfully amplified. FIG. 21B shows that 9 of the 12 PCR fractions having, on average, 1.08 DNA nanoballs as starting material were amplified.

### Sequence Analysis of Amplified Clonal Fractions

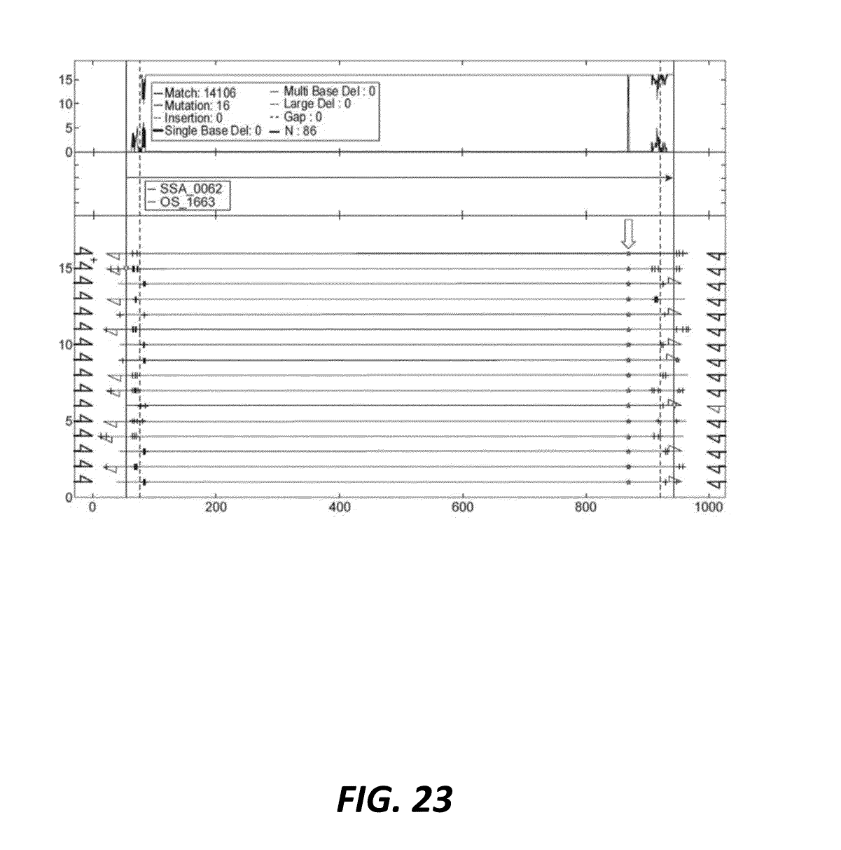

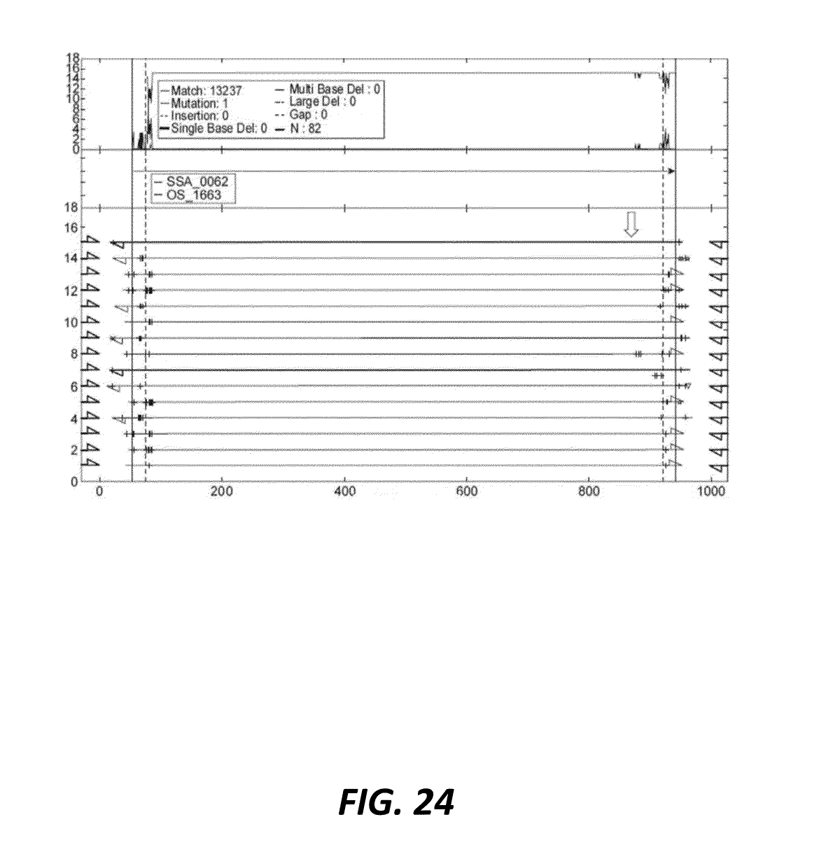

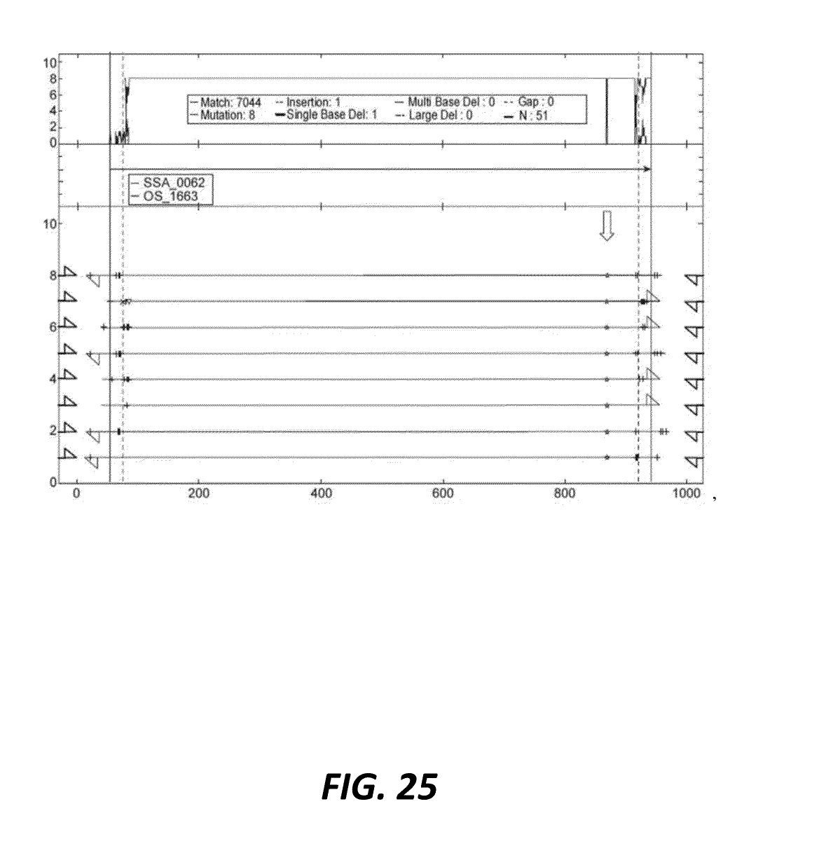

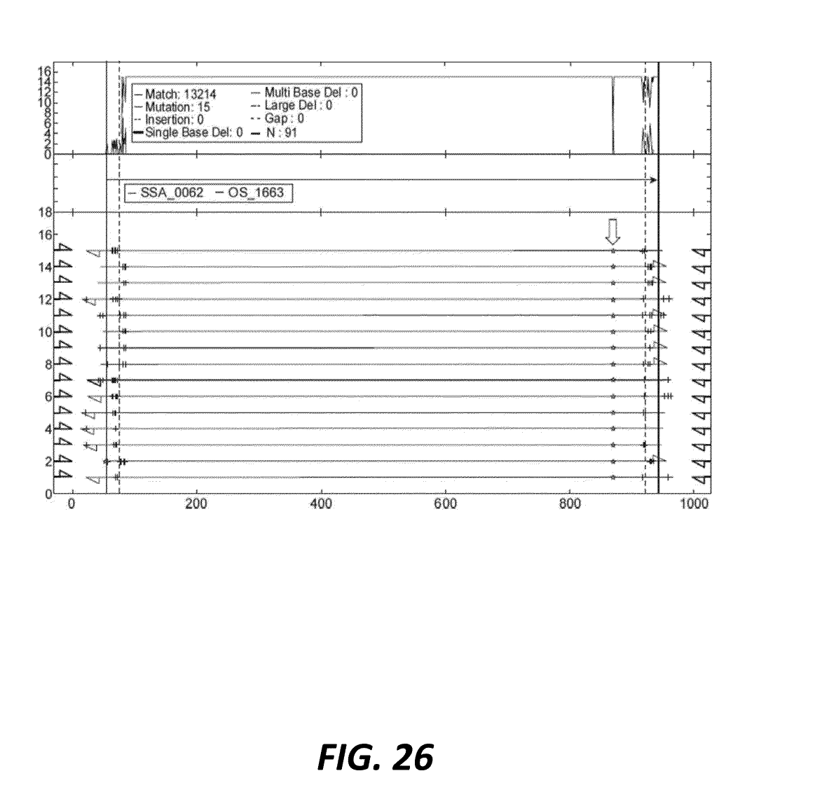

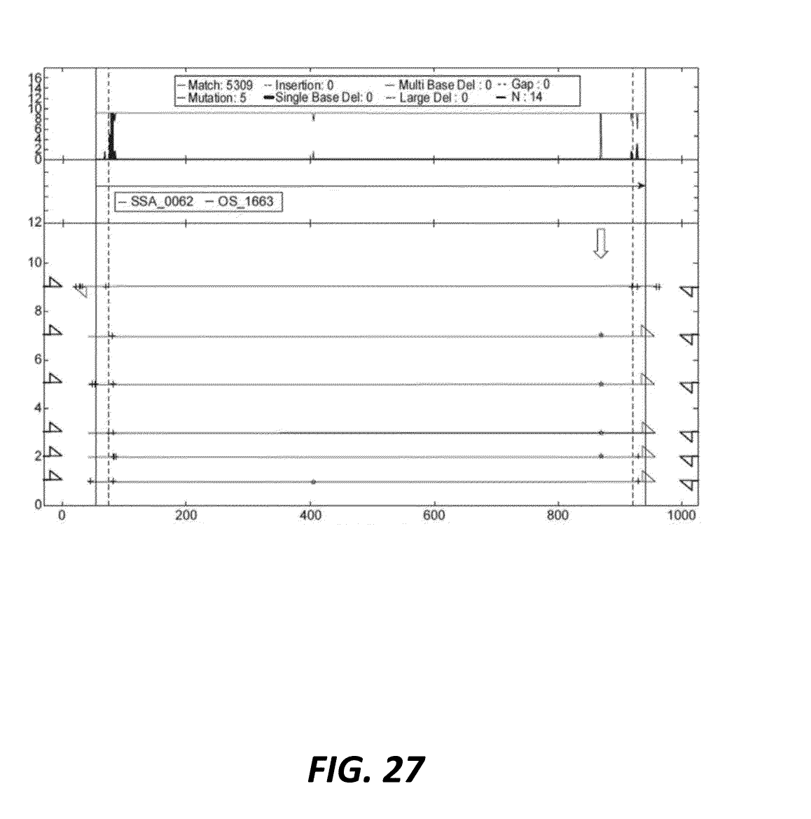

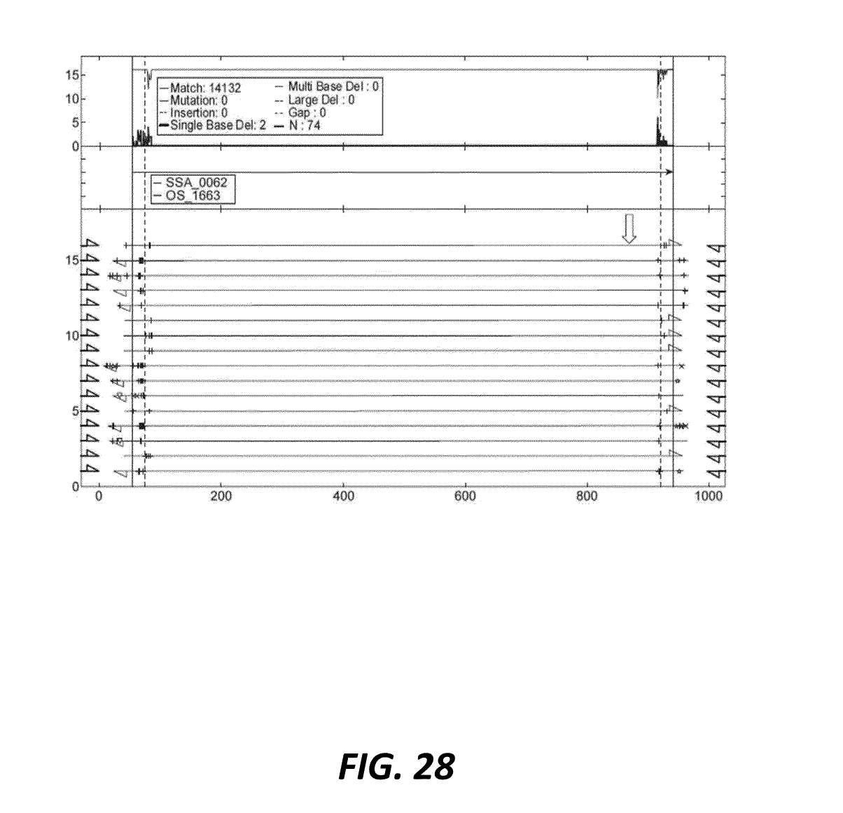

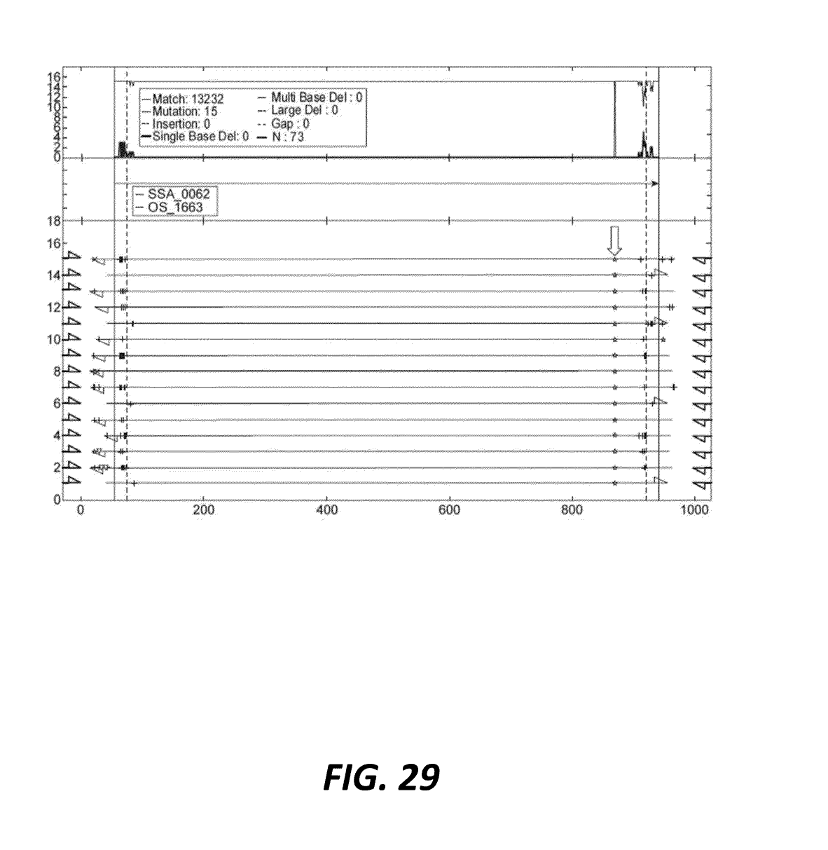

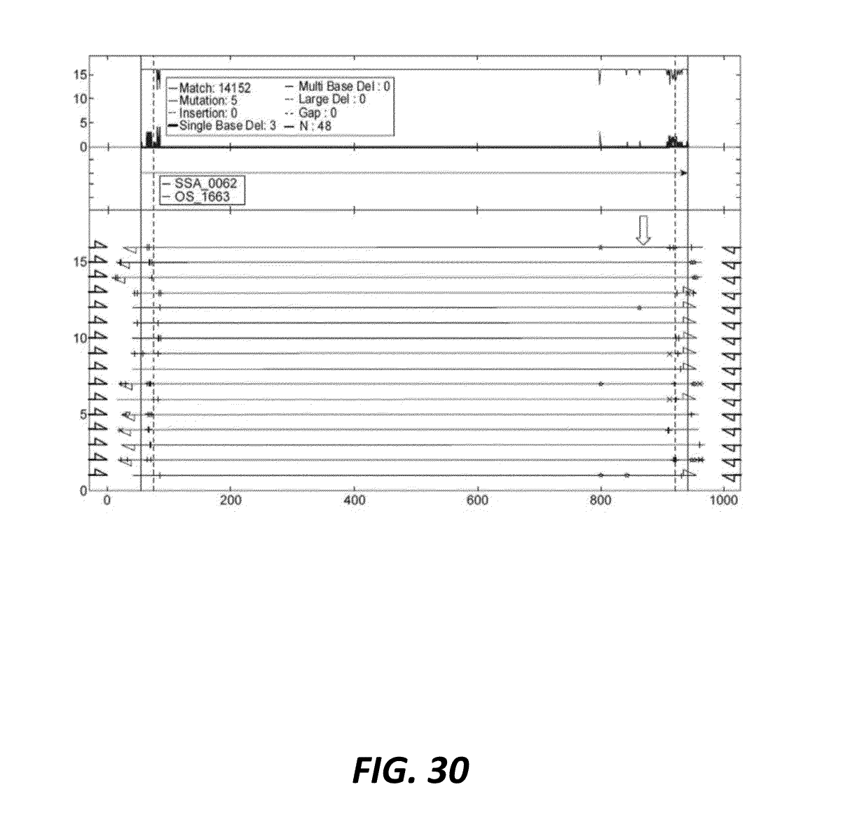

PCR amplification products from the clonal fractions were sequenced by Sanger sequencing. The sequence alignment maps for clonal fraction numbers 2, 3, 6, 7, 8, 9, 10, 11 and 12 (FIG. 21B) are shown in FIGS. 22-30, respectively. As shown in FIG. 22, fraction number 2 had a clonal population of nucleic acids without the C794T mutation (absence of asterisks in each sequence beneath the arrow). As shown in FIG. 23, fraction number 3 had a clonal population of nucleic acids with the C794T mutation, as indicated by the asterisks in each sequence located beneath the arrow. As shown in FIG. 24, fraction number 6 had a clonal population of nucleic acids without the C794T mutation (absence of asterisks in each sequence beneath the arrow). As shown in FIG. 25, fraction number 7 had a clonal population of nucleic acids with the C794T mutation, as indicated by the asterisks in each sequence located beneath the arrow. As shown in FIG. 26, fraction number 8 had a clonal population of nucleic acids with the C794T mutation, as indicated by the asterisks in each sequence located beneath the arrow. As shown in FIG. 27, 4 clones in fraction number 9 had a C794T mutation (asterisk under arrow) and 2 clones did not have the mutation (no asterisk under arrow). As shown in FIG. 28, fraction number 10 had a clonal population of nucleic acids without the C794T mutation (absence of asterisks in each sequence beneath the arrow). As shown in FIG. 29, fraction number 11 had a clonal population of nucleic acids with the C794T mutation, as indicated by the asterisks in each sequence located beneath the arrow. As shown in FIG. 30, fraction number 12 had a clonal population of nucleic acids without the C794T mutation (absence of asterisks in each sequence beneath the arrow). This example demonstrates a method for clonal sorting of a population of double-stranded DNA molecules via generation of bell like DNA. Amplification of the bell DNA by RCA resulted in DNA nanoballs that fold spontaneously, allowing for effective partitioning into single molecule fractions.

### Example 8

### Circularization of Target Nucleic Acids by Self-Ligation

Target nucleic acids were circularized by self-ligation using sticky ends or blunt ends. The target nucleic acids used in this example were assembled oligonucleic acids synthesized using the methods and systems described herein. The target nucleic acids were about 1 kbp in size.

For sticky end ligation, small adapter nucleic acid sequences were added to both ends of target nucleic acids to generate sticky ends. The addition of small adapter nucleic acid sequences was accomplished by amplification of the target nucleic acids with uracil containing primers, followed by treatment of the amplification products with a mixture of UDG and EndoVIII. The target nucleic acids were incorporated with small adapters to generate overhangs of 4, 6, 8 and 10 bases on both sides of the targets. The overhangs were designed, as described in Example 3, so that upon self-ligation only one of the two strands would anneal to a continuous strand and the other strand would not anneal and comprise a gap. Target nucleic acids having 4, 6, 8 or 10 base pair overhangs were self-ligated and the treated with exonuclease to remove non-ligated nucleic acids. FIG. 31A shows an image of a DNA agarose gel of target nucleic acids having 4, 6, 8 or 10 base pair overhangs following ligation (lanes 2, 3, 4 and 5, respectively) and following exonuclease treatment (lanes 7, 8, 9 and 10, respectively). Control lanes 1 and 6 correspond to target nucleic acids that lacked the small adapter nucleic acid sequences. FIG. 31A shows the presence of circularized target nucleic acids in lanes 7, 8, 9 and 10 after treatment with exonuclease. In contrast, no bands are observable in control lane 6, demonstrating that, unlike the linear DNA, the circularized bands are protected from exonuclease cleavage. FIG. 31B shows a plot of the amplification fold for self-ligated circularized targets having gap sizes of 1, 2, 3, 4 or 5 bases. Amplification reactions resulted in higher yield in the two cases where gap size was 1 base.

For blunt end ligation, target nucleic acids were amplified by PCR with a first primer that had a 5′ phosphate and a second primer that lacked a 5′ phosphate. The first few bases of the second primer comprised phosphorothioated bonds. The PCR products were self-ligated to generate a continuous circularized strand base paired to a discontinuous strand having a nick. The ligation products were treated with exonuclease to remove non-circularized DNA. FIG. 31C shows a DNA gel of the target nucleic acids during different steps of blunt end self-ligation. Lane 1 shows the target nucleic acids after amplification by PCR. Lane 2 shows the target nucleic acids after self-ligation. Lane 3 shows the ligation products after treatment with Lambda exonuclease. Lane 4 shows the ligation products after treatment with Exonuclease V. The resulting circularized targets were amplified by RCA.

While specific embodiments have been shown and described herein, it will be apparent to those skilled in the art that such embodiments are provided by way of example only. Numerous variations, changes, and substitutions will now occur to those skilled in the art without departing from the disclosed embodiments. It should be understood that various alternatives to the embodiments described herein may be employed in practicing the invention.

What is claimed is:

1-93. (canceled)

94. A method for nucleic acid sorting comprising:
(a) providing a plurality of circular double-stranded nucleic acids, each of the plurality of circular double-stranded nucleic acids comprising a first strand that is a continuous circle and a second strand that comprises a gap, wherein the gap has a length of at least one base;
(b) diluting the plurality of circular double-stranded nucleic acids to a concentration of less than 100 nM;
(c) extending the second strand a first amplification reaction, wherein the first strand is a template strand, thereby forming a plurality of amplicon nucleic acids comprising a plurality of copies of the first strand; and
(d) partitioning such that on average there are 0.1 to 10 amplicon nucleic acids per fraction.

95. The method of claim 94, wherein the second strand is a primer in the first amplification reaction, and wherein the first amplification reaction is performed without an additional primer sequence.

96. The method of claim 94, wherein the second strand is at least about 500 bases in length.

97. The method of claim 94, wherein the plurality of circular double-stranded nucleic acids comprises at least about 100 nucleic acids comprising at least 500 bases in length.

98. The method of claim 97, wherein the second strand comprises a nucleic acid sequence that differs in at least 7 bases from another second strand in the plurality of circular double-stranded nucleic acids.

99. The method of claim 94, wherein the gap is 1 to 5 bases in length.

100. The method of claim 94, wherein the plurality of circular double-stranded nucleic acids is formed by ligating a double-stranded vector to a double-stranded non-circularized nucleic acid, and wherein the vector anneals to a 5′ end and a 3′ end of the double-stranded non-circularized nucleic acid.

101. The method of claim 100, wherein the non-circularized double-stranded nucleic acid or the double-stranded vector comprises a strand having 1 to 10 fewer bases than a complementary strand, and wherein the 1 to 10 fewer bases corresponds to the length of the gap in the circular double-stranded nucleic acid.

102. The method of claim 101, wherein the gap is formed at a juncture between the double-stranded vector and each of the plurality of non-circularized double-stranded nucleic acids.

103. The method of claim 100, wherein each of the plurality of non-circularized double-stranded nucleic acids comprises an overhang formed by excision of a non-canonical base positioned at an end of a single strand of a precursor non-circularized double-stranded nucleic acid.

104. The method of claim 103, wherein the non-canonical base is positioned 4 to 10 bases from the end of the single strand of the precursor non-circularized double-stranded nucleic acid.

105. The method of claim 103, wherein the non-canonical base is uracil.

106. The method of claim 103, wherein one of the strands of the non-circularized double-stranded nucleic acid or double-stranded vector lacks a 5′ phosphate.

107. The method of claim 94, wherein the plurality of circular double-stranded nucleic acids is diluted to a concentration of less than about 100 pM prior to extending the second strand of each of the circular double-stranded nucleic acids.

108. The method of claim 94, wherein partitioning comprises diluting the plurality of amplicon nucleic acids to about 0.3 to 1.5 amplicon nucleic acids per fraction.

109. The method of claim 94, comprising a second amplification reaction, wherein the second amplification reaction is performed after partitioning.

110. The method of claim 94, wherein the circular double-stranded nucleic acids are heat denatured prior to amplification.

111. A method for nucleic acid sorting comprising:
(a) providing a plurality of circular double-stranded nucleic acids, each of the plurality of circular double-stranded nucleic acids comprising a first strand that is a continuous circle and a second strand comprising a gap, wherein the gap has a length of at least one base;
(b) partitioning such that on average there are about 0.1 to 10 circular double-stranded nucleic acids from the plurality of circular double-stranded nucleic acids per fraction; and
(c) amplifying the partitioned circular double-stranded nucleic acids in the presence of a random primer to generate a plurality of amplicon nucleic acids, wherein the random primer comprises 4 to 8 bases in length.

112. The method of claim 111, comprising forming each circular double-stranded nucleic acid by ligating a double-stranded vector to a double-stranded non-circularized nucleic acid, wherein the vector anneals to a 5′ end and a 3′ end of the double-stranded non-circularized nucleic acid.

113. The method of claim 112, wherein the double-stranded non-circularized nucleic acid or the double-stranded vector comprises a strand lacking a 5′ phosphate.

114. The method of claim 112, wherein the double-stranded non-circularized nucleic acid or the double-stranded vector comprises a strand having 1 to 10 fewer bases than a complementary strand, wherein the 1 to 10 fewer bases corresponds to the length of the gap in the circular double-stranded nucleic acids.

115. The method of claim 111, wherein the gap 1 to 5 bases in length.

116. The method of claim 111, wherein partitioning comprises diluting such that on average there are about 0.5 to 2 of the circular double-stranded nucleic acids per fraction.

117. The method of claim 111, wherein partitioning comprises diluting such that on average there is about 1 of the circular double-stranded nucleic acids per fraction.

118. The method of claim 111, wherein partitioning comprises diluting to a concentration of about 1.5 to 17 of the circular double-stranded nucleic acids per 1 μl of solution.

119. The method of claim 111, wherein the plurality of circular double-stranded nucleic acids comprises at least 100 circular double-stranded nucleic acids at least 500 bases in length.

120. The method of claim 111, wherein the plurality of circular double-stranded nucleic acids comprises nucleic acids that differ in at least 7 bases.

121. A method for nucleic acid sorting comprising:
(a) forming a plurality of circular single-stranded nucleic acids by joining a double-stranded non-circularized nucleic acid and two adaptor sequences, wherein each of the two adaptor sequences encodes for a hairpin secondary structure;
(b) diluting the plurality of circular single-stranded nucleic acids to a concentration of at most 1 nM;
(c) amplifying the plurality of circular single-stranded nucleic acids in the presence of a primer having sequence complementary to one of the two adaptor sequences; and
(d) partitioning the amplification reaction such that on average there are 0.1 to 10 amplicon nucleic acids per fraction.


SEQUENCE LISTING (75 sequences)

SEQ ID NO: 1 - DNA, 112 bp, Artificial Sequence
  Description of Artificial Sequence Synthetic polynucleotide
cgggatccttatcgtcatcgtcgtacagatcccgacccatttgctgtccaccagtcatgctagccataccatgatgatgatgatgatgagaaccccgcattttttttttttt

SEQ ID NO: 2 - DNA, 62 bp, Artificial Sequence
  Description of Artificial Sequence Synthetic oligonucleotide
atgaccatgattacggattcactggccgtcgttttacaacgtcgtgactgggaaaaccctgg

SEQ ID NO: 3 - DNA, 72 bp, Artificial Sequence
  Description of Artificial Sequence Synthetic oligonucleotide
gccagctggcgaaagggggatgtgctgcaaggcgattaagttgggtaacgccagggttttcccagtcacgac

SEQ ID NO: 4 - DNA, 70 bp, Artificial Sequence
  Description of Artificial Sequence Synthetic oligonucleotide
ccccctttcgccagctggcgtaatagcgaagaggcccgcaccgatcgcccttcccaacagttgcgcagcc

SEQ ID NO: 5 - DNA, 69 bp, Artificial Sequence
  Description of Artificial Sequence Synthetic oligonucleotide
cggcaccgcttctggtgccggaaaccaggcaaagcgccattcgccattcaggctgcgcaactgttggga

SEQ ID NO: 6 - DNA, 70 bp, Artificial Sequence
  Description of Artificial Sequence Synthetic oligonucleotide
caccagaagcggtgccggaaagctggctggagtgcgatcttcctgaggccgatactgtcgtcgtcccctc

SEQ ID NO: 7 - DNA, 75 bp, Artificial Sequence
  Description of Artificial Sequence Synthetic oligonucleotide
gataggtcacgttggtgtagatgggcgcatcgtaaccgtgcatctgccagtttgaggggacgacgacagtatcgg

SEQ ID NO: 8 - DNA, 74 bp, Artificial Sequence
  Description of Artificial Sequence Synthetic oligonucleotide
cccatctacaccaacgtgacctatcccattacggtcaatccgccgtttgttcccacggagaatccgacgggttg

SEQ ID NO: 9 - DNA, 72 bp, Artificial Sequence
  Description of Artificial Sequence Synthetic oligonucleotide
gtctggccttcctgtagccagctttcatcaacattaaatgtgagcgagtaacaacccgtcggattctccgtg

SEQ ID NO: 10 - DNA, 73 bp, Artificial Sequence
  Description of Artificial Sequence Synthetic oligonucleotide
gctggctacaggaaggccagacgcgaattatttttgatggcgttaactcggcgtttcatctgtggtgcaacgg

SEQ ID NO: 11 - DNA, 74 bp, Artificial Sequence
  Description of Artificial Sequence Synthetic oligonucleotide
caggtcaaattcagacggcaaacgactgtcctggccgtaaccgacccagcgcccgttgcaccacagatgaaacg

SEQ ID NO: 12 - DNA, 72 bp, Artificial Sequence
  Description of Artificial Sequence Synthetic oligonucleotide
cgtttgccgtctgaatttgacctgagcgcatttttacgcgccggagaaaaccgcctcgcggtgatggtgctg

SEQ ID NO: 13 - DNA, 72 bp, Artificial Sequence
  Description of Artificial Sequence Synthetic oligonucleotide
gccgctcatccgccacatatcctgatcttccagataactgccgtcactccagcgcagcaccatcaccgcgag

SEQ ID NO: 14 - DNA, 77 bp, Artificial Sequence
  Description of Artificial Sequence Synthetic oligonucleotide
aggatatgtggcggatgagcggcattttccgtgacgtctcgttgctgcataaaccgactacacaaatcagcgatttc

SEQ ID NO: 15 - DNA, 78 bp, Artificial Sequence
  Description of Artificial Sequence Synthetic oligonucleotide
ctccagtacagcgcggctgaaatcatcattaaagcgagtggcaacatggaaatcgctgatttgtgtagtcggtttatg

SEQ ID NO: 16 - DNA, 75 bp, Artificial Sequence
  Description of Artificial Sequence Synthetic oligonucleotide
atttcagccgcgctgtactggaggctgaagttcagatgtgcggcgagttgcgtgactacctacgggtaacagttt

SEQ ID NO: 17 - DNA, 71 bp, Artificial Sequence
  Description of Artificial Sequence Synthetic oligonucleotide
aaaggcgcggtgccgctggcgacctgcgtttcaccctgccataaagaaactgttacccgtaggtagtcacg

SEQ ID NO: 18 - DNA, 70 bp, Artificial Sequence
  Description of Artificial Sequence Synthetic oligonucleotide
gcggcaccgcgcctttcggcggtgaaattatcgatgagcgtggtggttatgccgatcgcgtcacactacg

SEQ ID NO: 19 - DNA, 74 bp, Artificial Sequence
  Description of Artificial Sequence Synthetic oligonucleotide
gatagagattcgggatttcggcgctccacagtttcgggttttcgacgttcagacgtagtgtgacgcgatcggca

SEQ ID NO: 20 - DNA, 74 bp, Artificial Sequence
  Description of Artificial Sequence Synthetic oligonucleotide
gagcgccgaaatcccgaatctctatcgtgcggtggttgaactgcacaccgccgacggcacgctgattgaagcag

SEQ ID NO: 21 - DNA, 73 bp, Artificial Sequence
  Description of Artificial Sequence Synthetic oligonucleotide
cagcagcagaccattttcaatccgcacctcgcggaaaccgacatcgcaggcttctgcttcaatcagcgtgccg

SEQ ID NO: 22 - DNA, 74 bp, Artificial Sequence
  Description of Artificial Sequence Synthetic oligonucleotide
cggattgaaaatggtctgctgctgctgaacggcaagccgttgctgattcgaggcgttaaccgtcacgagcatca

SEQ ID NO: 23 - DNA, 75 bp, Artificial Sequence
  Description of Artificial Sequence Synthetic oligonucleotide
gcaggatatcctgcaccatcgtctgctcatccatgacctgaccatgcagaggatgatgctcgtgacggttaacgc

SEQ ID NO: 24 - DNA, 75 bp, Artificial Sequence
  Description of Artificial Sequence Synthetic oligonucleotide
cagacgatggtgcaggatatcctgctgatgaagcagaacaactttaacgccgtgcgctgttcgcattatccgaac

SEQ ID NO: 25 - DNA, 74 bp, Artificial Sequence
  Description of Artificial Sequence Synthetic oligonucleotide
tccaccacatacaggccgtagcggtcgcacagcgtgtaccacagcggatggttcggataatgcgaacagcgcac

SEQ ID NO: 26 - DNA, 75 bp, Artificial Sequence
  Description of Artificial Sequence Synthetic oligonucleotide
gctacggcctgtatgtggtggatgaagccaatattgaaacccacggcatggtgccaatgaatcgtctgaccgatg

SEQ ID NO: 27 - DNA, 73 bp, Artificial Sequence
  Description of Artificial Sequence Synthetic oligonucleotide
gcaccattcgcgttacgcgttcgctcatcgccggtagccagcgcggatcatcggtcagacgattcattggcac

SEQ ID NO: 28 - DNA, 73 bp, Artificial Sequence
  Description of Artificial Sequence Synthetic oligonucleotide
cgcgtaacgcgaatggtgcagcgcgatcgtaatcacccgagtgtgatcatctggtcgctggggaatgaatcag

SEQ ID NO: 29 - DNA, 78 bp, Artificial Sequence
  Description of Artificial Sequence Synthetic oligonucleotide
ggatcgacagatttgatccagcgatacagcgcgtcgtgattagcgccgtggcctgattcattccccagcgaccagatg

SEQ ID NO: 30 - DNA, 72 bp, Artificial Sequence
  Description of Artificial Sequence Synthetic oligonucleotide
gtatcgctggatcaaatctgtcgatccttcccgcccggtgcagtatgaaggcggcggagccgacaccacggc

SEQ ID NO: 31 - DNA, 71 bp, Artificial Sequence
  Description of Artificial Sequence Synthetic oligonucleotide
cgggaagggctggtcttcatccacgcgcgcgtacatcgggcaaataatatcggtggccgtggtgtcggctc

SEQ ID NO: 32 - DNA, 73 bp, Artificial Sequence
  Description of Artificial Sequence Synthetic oligonucleotide
tggatgaagaccagcccttcccggctgtgccgaaatggtccatcaaaaaatggctttcgctacctggagagac

SEQ ID NO: 33 - DNA, 73 bp, Artificial Sequence
  Description of Artificial Sequence Synthetic oligonucleotide
ccaagactgttacccatcgcgtgggcgtattcgcaaaggatcagcgggcgcgtctctccaggtagcgaaagcc

SEQ ID NO: 34 - DNA, 73 bp, Artificial Sequence
  Description of Artificial Sequence Synthetic oligonucleotide
cgcgatgggtaacagtcttggcggtttcgctaaatactggcaggcgtttcgtcagtatccccgtttacagggc

SEQ ID NO: 35 - DNA, 78 bp, Artificial Sequence
  Description of Artificial Sequence Synthetic oligonucleotide
gccgttttcatcatatttaatcagcgactgatccacccagtcccagacgaagccgccctgtaaacggggatactgacg

SEQ ID NO: 36 - DNA, 76 bp, Artificial Sequence
  Description of Artificial Sequence Synthetic oligonucleotide
cagtcgctgattaaatatgatgaaaacggcaacccgtggtcggcttacggcggtgattttggcgatacgccgaacg

SEQ ID NO: 37 - DNA, 67 bp, Artificial Sequence
  Description of Artificial Sequence Synthetic oligonucleotide
gcggcgtgcggtcggcaaagaccagaccgttcatacagaactggcgatcgttcggcgtatcgccaaa

SEQ ID NO: 38 - DNA, 71 bp, Artificial Sequence
  Description of Artificial Sequence Synthetic oligonucleotide
cgaccgcacgccgcatccagcgctgacggaagcaaaacaccagcagcagtttttccagttccgtttatccg

SEQ ID NO: 39 - DNA, 79 bp, Artificial Sequence
  Description of Artificial Sequence Synthetic oligonucleotide
ctcgttatcgctatgacggaacaggtattcgctggtcacttcgatggtttgcccggataaacggaactggaaaaactgc

SEQ ID NO: 40 - DNA, 75 bp, Artificial Sequence
  Description of Artificial Sequence Synthetic oligonucleotide
aatacctgttccgtcatagcgataacgagctcctgcactggatggtggcgctggatggtaagccgctggcaagcg

SEQ ID NO: 41 - DNA, 76 bp, Artificial Sequence
  Description of Artificial Sequence Synthetic oligonucleotide
gttcaggcagttcaatcaactgtttaccttgtggagcgacatccagaggcacttcaccgcttgccagcggcttacc

SEQ ID NO: 42 - DNA, 78 bp, Artificial Sequence
  Description of Artificial Sequence Synthetic oligonucleotide
caaggtaaacagttgattgaactgcctgaactaccgcagccggagagcgccgggcaactctggctcacagtacgcgta

SEQ ID NO: 43 - DNA, 71 bp, Artificial Sequence
  Description of Artificial Sequence Synthetic oligonucleotide
gcgctgatgtgcccggcttctgaccatgcggtcgcgttcggttgcactacgcgtactgtgagccagagttg

SEQ ID NO: 44 - DNA, 67 bp, Artificial Sequence
  Description of Artificial Sequence Synthetic oligonucleotide
ccgggcacatcagcgcctggcagcagtggcgtctggcggaaaacctcagtgtgacgctccccgccgc

SEQ ID NO: 45 - DNA, 71 bp, Artificial Sequence
  Description of Artificial Sequence Synthetic oligonucleotide
ccagctcgatgcaaaaatccatttcgctggtggtcagatgcgggatggcgtgggacgcggcggggagcgtc

SEQ ID NO: 46 - DNA, 78 bp, Artificial Sequence
  Description of Artificial Sequence Synthetic oligonucleotide
cgaaatggatttttgcatcgagctgggtaataagcgttggcaatttaaccgccagtcaggctttctttcacagatgtg

SEQ ID NO: 47 - DNA, 74 bp, Artificial Sequence
  Description of Artificial Sequence Synthetic oligonucleotide
tgaactgatcgcgcagcggcgtcagcagttgttttttatcgccaatccacatctgtgaaagaaagcctgactgg

SEQ ID NO: 48 - DNA, 73 bp, Artificial Sequence
  Description of Artificial Sequence Synthetic oligonucleotide
gccgctgcgcgatcagttcacccgtgcaccgctggataacgacattggcgtaagtgaagcgacccgcattgac

SEQ ID NO: 49 - DNA, 71 bp, Artificial Sequence
  Description of Artificial Sequence Synthetic oligonucleotide
ggcctggtaatggcccgccgccttccagcgttcgacccaggcgttagggtcaatgcgggtcgcttcactta

SEQ ID NO: 50 - DNA, 70 bp, Artificial Sequence
  Description of Artificial Sequence Synthetic oligonucleotide
cgggccattaccaggccgaagcagcgttgttgcagtgcacggcagatacacttgctgatgcggtgctgat

SEQ ID NO: 51 - DNA, 75 bp, Artificial Sequence
  Description of Artificial Sequence Synthetic oligonucleotide
tccggctgataaataaggttttcccctgatgctgccacgcgtgagcggtcgtaatcagcaccgcatcagcaagtg

SEQ ID NO: 52 - DNA, 78 bp, Artificial Sequence
  Description of Artificial Sequence Synthetic oligonucleotide
ggggaaaaccttatttatcagccggaaaacctaccggattgatggtagtggtcaaatggcgattaccgttgatgttga

SEQ ID NO: 53 - DNA, 75 bp, Artificial Sequence
  Description of Artificial Sequence Synthetic oligonucleotide
ggcagttcaggccaatccgcgccggatgcggtgtatcgctcgccacttcaacatcaacggtaatcgccatttgac

SEQ ID NO: 54 - DNA, 71 bp, Artificial Sequence
  Description of Artificial Sequence Synthetic oligonucleotide
gcggattggcctgaactgccagctggcgcaggtagcagagcgggtaaactggctcggattagggccgcaag

SEQ ID NO: 55 - DNA, 72 bp, Artificial Sequence
  Description of Artificial Sequence Synthetic oligonucleotide
ggcagatcccagcggtcaaaacaggcggcagtaaggcggtcgggatagttttcttgcggccctaatccgagc

SEQ ID NO: 56 - DNA, 72 bp, Artificial Sequence
  Description of Artificial Sequence Synthetic oligonucleotide
gttttgaccgctgggatctgccattgtcagacatgtataccccgtacgtcttcccgagcgaaaacggtctgc

SEQ ID NO: 57 - DNA, 69 bp, Artificial Sequence
  Description of Artificial Sequence Synthetic oligonucleotide
gtcgccgcgccactggtgtgggccataattcaattcgcgcgtcccgcagcgcagaccgttttcgctcgg

SEQ ID NO: 58 - DNA, 72 bp, Artificial Sequence
  Description of Artificial Sequence Synthetic oligonucleotide
accagtggcgcggcgacttccagttcaacatcagccgctacagtcaacagcaactgatggaaaccagccatc

SEQ ID NO: 59 - DNA, 75 bp, Artificial Sequence
  Description of Artificial Sequence Synthetic oligonucleotide
gaaaccgtcgatattcagccatgtgccttcttccgcgtgcagcagatggcgatggctggtttccatcagttgctg

SEQ ID NO: 60 - DNA, 71 bp, Artificial Sequence
  Description of Artificial Sequence Synthetic oligonucleotide
catggctgaatatcgacggtttccatatggggattggtggcgacgactcctggagcccgtcagtatcggcg

SEQ ID NO: 61 - DNA, 74 bp, Artificial Sequence
  Description of Artificial Sequence Synthetic oligonucleotide
ttatttttgacaccagaccaactggtaatggtagcgaccggcgctcagctggaattccgccgatactgacgggc

SEQ ID NO: 62 - DNA, 3075 bp, Artificial Sequence
  Description of Artificial Sequence Synthetic polynucleotide
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 63 - DNA, 27 bp, Artificial Sequence
  Description of Artificial Sequence Synthetic primer
atgaccatgattacggattcactggcc

SEQ ID NO: 64 - DNA, 31 bp, Artificial Sequence
  Description of Artificial Sequence Synthetic primer
ttatttttgacaccagaccaactggtaatgg

SEQ ID NO: 65 - DNA, 1042 bp, Artificial Sequence
  Description of Artificial Sequence Synthetic polynucleotide
cagcagttcctcgctcttctcacgacgagttcgacatcaacaagctgcgctaccacaagatcgtgctgatggccgacgccgatgttgacggccagcacatcgcaacgctgctgctcaccctgcttttccgcttcatgccagacctcgtcgccgaaggccacgtctacttggcacagccacctttgtacaaactgaagtggcagcgcggagagccaggattcgcatactccgatgaggagcgcgatgagcagctcaacgaaggccttgccgctggacgcaagatcaacaaggacgacggcatccagcgctacaagggtctcggcgagatgaacgccagcgagctgtgggaaaccaccatggacccaactgttcgtattctgcgccgcgtggacatcaccgatgctcagcgtgctgatgaactgttctccatcttgatgggtgacgacgttgtggctcgccgcagcttcatcacccgaaatgccaaggatgttcgtttcctcgatatctaaagcgccttacttaacccgcccctggaattctgggggcgggttttgtgatttttagggtcagcactttataaatgcaggcttctatggcttcaagttggccaatacgtggggttgattttttaaaaccagactggcgtgcccaagagctgaactttcgctagtcatgggcattcctggccggtttcttggccttcaaaccggacaggaatgcccaagttaacggaaaaaccgaaagaggggcacgccagtctggttctcccaaactcaggacaaatcctgcctcggcgcctgcgaaaagtgccctctcctaaatcgtttctaagggctcgtcagaccccagttgatacaaacatacattctgaaaattcagtcgcttaaatgggcgcagcgggaaatgctgaaaactacattaatcaccgataccctagggcacgtgacctctactgaacccaccaccacagcccatgttccactacctgatggatcttccactccagtccaaatttgggcgtacactgcgagtccactacgat

SEQ ID NO: 66 - DNA, 20 bp, Artificial Sequence
  Description of Artificial Sequence Synthetic primer
cagcagtucctcgctcttct

SEQ ID NO: 67 - DNA, 22 bp, Artificial Sequence
  Description of Artificial Sequence Synthetic primer
atcgtaguggactcgcagtgta

SEQ ID NO: 68 - DNA, 31 bp, Artificial Sequence
  Description of Artificial Sequence Synthetic oligonucleotide
tacgctcttcctcagcagtggtcatcgtagt

SEQ ID NO: 69 - DNA, 30 bp, Artificial Sequence
  Description of Artificial Sequence Synthetic oligonucleotide
accactgctgaggaagagcgtacagcagtt

SEQ ID NO: 70 - DNA, 20 bp, Artificial Sequence
  Description of Artificial Sequence Synthetic primer
cagcagttcctcgctcttct

SEQ ID NO: 71 - DNA, 22 bp, Artificial Sequence
  Description of Artificial Sequence Synthetic primer
atcgtagtggactcgcagtgta

SEQ ID NO: 72 - DNA, 49 bp, Artificial Sequence
  Description of Artificial Sequence Synthetic oligonucleotide
ctctctcttttcctcctcctccgttgttgttgttgagagagtcgactgt

SEQ ID NO: 73 - DNA, 49 bp, Artificial Sequence
  Description of Artificial Sequence Synthetic oligonucleotide
gagctgccccaccatccacccgtatctcatccaagcagctcctgttgct

SEQ ID NO: 74 - DNA, 12 bp, Artificial Sequence
  Description of Artificial Sequence Synthetic primer
ggaggaggagga

SEQ ID NO: 75 - DNA, 12 bp, Artificial Sequence
  Description of Artificial Sequence Synthetic primer
gatacgggtgga